(19) United States
(12) Patent Application Publication
(10) Pub. No.: US 2026/0232730 A1
(43) Pub. Date: Aug. 13, 2026

(54) TUNING EXPRESSION FOR IMMUNE OR CANCER THERAPIES

(71) Applicants: The J. David Gladstone Institutes, a testamentary trust established under the Will of J. David, San Francisco, CA (US); The Regents of The University of California, Oakland, CA (US)

(72) Inventors: Jennifer M. Umhoefer et al., San Francisco, CA (US); Alexander Marson, San Francisco, CA (US); Maya M. Arce, San Francisco, CA (US)

(21) Appl. No.: 19/152,870

(22) PCT Filed: Feb. 16, 2024
(86) PCT No.: PCT/US2024/016301

§ 371 Date: Jul. 31, 2025

Related U.S. Application Data
(60) Provisional application No. 63/446,247, filed on Feb. 16, 2023.

(51) Int. Cl. A61K 35/17; A61K 35/12; A61P 35/00; C12N 5/0783; C12N 9/22; C12N 15/11; C12N 15/86

(52) U.S. Cl. CPC A61K 35/17; A61P 35/00; C12N 5/0637; C12N 9/222; C12N 15/111; C12N 15/86; A61K 2035/122; C12N 2310/20

(57) ABSTRACT

Methods to identify modified Treg cells, compositions comprising those cells or gRNAs, and methods of using those cells are provided.

### PRIORITY APPLICATION

This application claims benefit of priority to the filing date of U.S. Provisional Application Ser. No. 63/446,247, filed Feb. 16, 2023, the contents of which are specifically incorporated herein by reference in their entireties.

### BACKGROUND

Tregs play an indispensable role in establishing and maintaining immune tolerance. Over the life of an individual, subsets of these cells accumulate in nonlymphoid organs, with a predilection to stably reside in barrier tissues, such as skin, lung, and the gastrointestinal tract. In addition, populations of Tregs can be found in visceral adipose tissue, skeletal muscle, and heart. A growing body of research suggests that tissue Tregs have unique functions that are largely dependent on the specific organs in which they reside. Treg production of amphiregulin within skeletal muscle, lung, and brain facilitates normal tissue regeneration after injury, whereas adipose tissue Tregs are crucial in attenuating inflammatory processes in fat and maintaining insulin sensitivity. IgA selection is specifically regulated by a subset of Tregs in the colon, whereas skin Tregs facilitate full-thickness wound healing, epidermal repair, and regulate hair follicle cycling.

Implicit in the observation that Tregs have different functions in different tissues is the idea that these cells take cues from and adapt to their local environment. Perhaps the cellular process that is most influenced by the local tissue environment is metabolism. Oxygen and nutrient availability vary greatly between healthy tissues and change profoundly during disease. Tregs in the gastrointestinal tract are influenced by specific bacterial-derived metabolites, whereas prolyl hydroxylase protein expression in the lungs induces Tregs in response to changing oxygen tension, a process exacerbated by lung cancer metastasis. Adipose tissue Tregs both sense and metabolize lipids through a distinct transcriptional program, including LDLR, Dgat, and Pgatl. Thus, tissue Tregs respond to signals from their local environment and acquire specific metabolic programs to both survive and optimally mediate their functions in the face of dynamically changing nutrient availability and oxygen tension.

One major sensor integrating multiple environmental cues is mTOR, a key regulator of cell growth and driver of metabolic activity. Whereas in many immunological contexts, mTOR promotes proliferation and differentiation, Tregs use mTOR signaling, mediated by 2 separate complexes (mTORC1 and mTORC2), as a rheostat to modulate and balance growth and suppressive function. Signaling mediated by mTORC1 inhibits Treg expansion and differentiation, as the prototypical mTORC1 inhibitor rapamycin restricts these processes in vitro and in vivo. However, absolute loss of mTORC1 in Tregs leads to widespread autoimmunity, whereas mTORC2 signaling appears to be dispensable for Treg function. Thus, Tregs residing in peripheral tissues must dynamically regulate signaling through the mTORC1 pathway, requiring some degree of mTORC1 signaling for survival but reducing excessive signaling to allow for proliferation. Indeed, tissue Treg expression of cMaf has recently been described as a means by which Tregs residing in the colon intrinsically suppress mTOR activity.

### SUMMARY

Methods of boosting (or inhibiting) FOXP3 expression in Tregs are disclosed herein, which methods are useful to engineer improved cell therapies. CRISPR interference (CRISPRi) and CRISPR nuclease (CRISPRn) genetic screens in human regulatory T cells (Tregs) and non-Treg CD4+ T cells (Teff) were employed to identify cis- and trans-regulators of FOXP3 expression. By tiling CRISPRi machinery across approximately 95 kb of the FOXP3 locus, positive and negative cis regulators of FOXP3 expression were identified. In one embodiment, a noncoding region upstream of the FOXP3 promoter involved in FOXP3 suppression was identified. This region, referred to as FLICR, maps to the human homologue of Flicr, a lncRNA previously described in murine Tregs (Zemmour, D. et al. PNAS 2017). Specific guide RNAs targeting this locus can be introduced into human Tregs to increase FOXP3 expression. Cis-regulators were also tested using arrayed CRISPRn deletion of elements which confirmed the FOXP3-suppressive capacity of FLICR. Additionally, CRISPRn screens targeting 1350 human transcription factors and immune genes yielded significant positive and negative trans-regulators of FOXP3 expression in Tregs and Teff. To assess the capacity of these cis- and trans-regulators to maintain Treg identity under strong inflammation, those regulators were knocked out (KO) or deleted in human Tregs and then exposed to repetitive CD28/CD3/CD2 stimulation and inflammatory cytokines. KO or deletion of multiple regulators involved in FOXP3 suppression, including, for example, SRF, VARS2, and TFDP1, increased the percentage of Tregs maintaining FOXP3 and HELIOS expression and decreased the percentage losing FOXP3 and HELIOS expression relative to AAVS1-targeted controls. Methods are disclosed to improve Treg function and stability which are useful in therapies for autoimmunity or to selectively destabilize Tregs in therapies for cancer or infectious diseases.

For example, positive and negative cis- and trans-regulators of FOXP3 expression can be selectively perturbed in Tregs which in turn may be used to (1) improve Treg stability, and potentially function, for autoimmune treatment applications, or (2) selectively destabilize Tregs in a cancer setting to enable enhanced immune recognition of tumors. Perturbation of these targets can be genetically implemented in Treg cellular immunotherapies or as small molecular therapeutics targeting endogenous Tregs.

Genetic editing of the regulators in Treg cellular immunotherapies gives rise to Treg therapeutics with enhanced stability, and therefore safety and function, in autoimmune and transplant tolerance settings. Additionally, genetic editing or small-molecule targeting of the regulators in human Tregs in a cancer setting can induce selective functional inactivity of tumor-associated Tregs to enable enhanced immune recognition and clearance of tumors, thereby enhancing cancer immunotherapies by disrupting Tregs.

The gRNAs or modified Treg cells can be used in conjunction with other Treg cellular therapies or cancer therapeutics.

One embodiment provides a method to identify regulators of Treg cells, comprising: contacting human Treg cells with one or more gRNAs targeted to one or more coding or non-coding regions in one or more genes and a polypeptide comprising a Cas polypeptide or nucleic acid encoding the polypeptide; and selecting one or more human Treg cells that have an altered activity and optionally isolating the Treg cells with altered activity. In one embodiment, the gRNAs are targeted to RUNX3, CBFB, DNMT1, E2F3, EGR2, EGR3, ETS1, FOXO1, GABPA, GATA3, HIC1, HIF1A, HSF2, FOXN2, IL2RA, IRF2, IRF4, SMAD4, MTF1, NFKB2, YBX1, MAP2K1, PTEN, RELA, SATB1, SRF, STAT5A, STAT5B, TFDP1, TGFBR1, YY1, ZNF143, NR4A3, MBD2, MED14, SETDB1, MED12, IKZF1, CTCF, GMEB1, IKZF3, MTF2, MGA, USP22, ZMYND8, ZBTB32, FOXP1, TAF5L, TBX21, FOXP3, ATXN7L3, VARS2, ZNF574, BCL11B, MED30, TUBB, ZNF740, or MED11. In one embodiment, the gRNAs comprise one or more of SEQ ID NOs: 102-379 or 402-424. Also provided is a population of cells obtained by the method (including editing in CD4 T cells that are induced to be Treg cells).

In one embodiment, an isolated human Treg having altered FOXP3 activity as a result of a genetic modification, e.g., associated with a gRNA, is provided. In one embodiment, the altered activity is enhanced activity. In one embodiment, the altered activity is decreased activity. Before the genetic modification, the isolated human Treg may be obtained from a patient with cancer or an autoimmune disease.

Genetic modifications include but are not limited to insertions of one or more nucleotides, substitutions of one or more nucleotides, deletions of one or more nucleotides, or any combination thereof. Genetic modifications can also include modifying the epigenome with CRISPR technologies including CRISPRi, CRISPRa, CRISPRon, and CRISPRoff.

In one embodiment, a method to prevent, inhibit or treat cancer in a mammal is provided comprising administering to the mammal a composition having a plurality of Treg cells having a genetic modification, e.g., a cis- or trans-modification as disclosed herein.

In one embodiment, a method to prevent, inhibit or treat an autoimmune disease in a mammal is provided comprising administering to the mammal a composition having a plurality of Treg cells having a genetic modification, e.g., a cis- or trans-modification as disclosed herein.

### DESCRIPTION OF THE FIGURES

Many aspects of the present disclosure can be better understood with reference to the following drawings. The components in the drawings are not necessarily to scale. Instead, emphasis is placed on clearly illustrating the principles of the present disclosure. Furthermore, components can be shown as transparent in certain views for clarity of illustration only and not to indicate that the illustrated component is necessarily transparent.

FIG. 1 illustrates engineering Treg cell therapies (adapted from Ferreira et al., Nat. Rev. Drug Discov (2019)).

FIG. 2 illustrates that Treg stability in humans has implications for cancer and autoimmunity (adapted from Lucca et al., Nat Rev Immunol. (2020).

FIG. 3 illustrates gene networks are comprised of trans- and cis regulators.

FIG. 4 provides an overview of a CRISPRi cis-regulatory screen (adapted from Schmidt, R & Steinhart, Z et al. Science (2022)).

FIG. 5 illustrates a CRISPRi cis-regulatory screen (adapted from Schmidt, R & Steinhart, Z et al. Science (2022)).

FIG. 6 illustrates CRISPRi tiling screen identifying FOXP3 cis-regulatory elements.

FIG. 7 provides FOXP3 cis-regulatory elements across the FOXP3 locus. Each point is a group of neighboring sgRNAs. Points above 0 indicate FOXP3 suppressors, and those that fall below the 0 are FOXP3 maintenance factors.

FIG. 8 illustrates results of a tiling screen identifying CNSs as maintenance cis regulators of FOXP3.

FIG. 9 illustrates other sites of interest.

FIG. 10 illustrates that FLICR is specifically expressed in Tregs, most notably thymus derived Tregs (adapted from Zemmour et al., PNAS (2017)).

FIG. 11 shows that FLICR KO resulted in slightly increased Foxp3 MFI. A deletion of a portion of a FLICR exon in mice resulted in a decreased % of Foxp3 low cells and a modest increase in FOXP3 MFI (adapted from Zemmour et al., PNAS (2017)).

FIG. 12 illustrates genomic element deletion with paired CRISPR gRNAs. Paired CRISPR gRNAs were used to excise the region.

FIG. 13 illustrates CRISPRi hits with paired RNP deletions.

FIG. 14 illustrates CRISPRi hits with paired RNP deletions: Day 9 with a representative flow cytometry plot.

FIG. 15 shows gene networks are comprised of trans- and cis regulators.

FIG. 16 illustrates CRISPRn trans-regulator screen overview (adapted from Shifrut, E & Carnevale, J et al. Cell (2018)).

FIG. 17 illustrates trans-regulators of FOXP3 in human Tregs.

FIG. 18 illustrates assessing efficacy in a xeno-GvHD model (adapted from Schumann et al., Nat Immunol (2020)).

FIG. 19 illustrates FLICR is specifically expressed in human Tregs (adapted from Zemmour et al., PNAS (2017)).

FIG. 20 illustrates some conservation in FLICR between humans and mice (adapted from Zemmour et al., PNAS (2017)).

FIG. 21 illustrates Donor 1 Treg Editing Efficiency. CRISPR-deleted regions were amplified using PCR primers outside of the region of interest. The size of the resulting amplified fragments were analyzed using gel electrophoresis. The ratio of the lower weight (small) gel band (indicating region deletion) to the higher weight (large) gel band (indicating unsuccessful deletion) indicates the extent of deletion efficiency. The top portion of the gel contains samples amplified from edited genomic DNA, while the bottom is from unedited genomic DNA. The first line of text is the gRNA ID(s) and indicates the identity of the sample below. The next line of text indicates the expected size of the unedited fragment (above) or the edited fragment (below). Samples receiving a single gRNA are also shown here, but editing efficiency cannot be assessed with this method.

FIG. 22 illustrates FOXP3 is a marker of Treg stability (adapted from Lu et al., Nat Rev Immunol. (2017).

FIG. 23 illustrates FOXP3 expression peaks approximately 24-48 hours post-activation in human Tregs and approximately 48 hours post-activation in human CD4+ T cells (Teffs).

FIG. 24 illustrates conservative sorting of Tregs to eliminate Teff contaminants.

FIG. 25 illustrates trans-regulators of FOXP3 in non-Treg CD4+ T cells.

FIG. 26 provides sequences for CNS0-3 (SEQ ID NOs: 21-23), which are regions that are enhancers of FOXP3 expression. CNS0 and NCNS (SEQ ID NO: 20) are regions involved in maintenance of FOXP3 expression in human Teff.

FIG. 27 illustrates trans regulatory gRNA sequences (SEQ ID NOs: 102-379; published gRNA library named the Brunello library and described in Sanson, K. R., et al., Nat. Commun (2018)).

FIG. 28 illustrates cis regulatory gRNA sequences (SEQ ID NOS: 402-424; Tregs SEQ ID NOs: 402-413).

FIG. 29 illustrates cis regulatory gRNA pairs (SEQ ID NOs: 425-446) (indicating which gRNAs from FIG. 28 are used together to excise cis regions, these gRNAs can be combined in other pairs with each other to excise cis elements).

FIGS. 30A-30D illustrate that CRISPRi tiling screen identifies cis-regulators of FOXP3 in human Tregs. A. Schematic depicting CRISPRi-based screens for FOXP3 cis-regulators. Schematic adapted from Schmidt, R., and Steinhart, Z., et al., Science 2022. B. Absolute value of the Log2 fold change of sgRNA enrichment in FOXP3 high vs. low FACS bins plotted along the FOXP 3 locus. C. Arrayed validation of FLICR region-associated CRISPRi-responsive elements with paired Cas9 RNPs plotted along the FOXP 3 locus in resting and stimulated Tregs. Width of bars indicate cutting location of Cas9 RNPs. D. Representative flow plots of excised FLICR region-associated CRISPRi-responsive elements.

FIGS. 31A-31D illustrate that CRISPRn transcription factor screens identify trans-regulators of FOXP3 in human Tregs. A. Schematic depicting CRISPRn-based screens for FOXP3 trans-regulators. Schematic adapted from Shifrut, E., and Carnevale, J., et al., Cell 2018. B. Volcano plot of Log2 fold change of sgRNA enrichment in FOXP3 high vs. low FACS bins versus −Log10 of p-value. Hits associated with a FDR <0.05 are colored. C. Density plot of Log2 fold change of individual sgRNA enrichment in FOXP3 high vs. low FACS bins for the top 5 FOXP3 maintenance and suppressive regulators. D. Arrayed validation of top FOXP3 maintenance and suppressive regulators in resting and stimulated Tregs.

FIGS. 32A-32C demonstrate that FOXP3-suppressive regulators maintain increased FOXP3 expression under challenge. A. Log2 fold change in FOXP3 MFI of KO versus AAVS1-targeting sgRNAs across Treg challenge conditions. Absence of Restimulation Tregs were cultured for 18 days following initial stimulation for editing. Low IL-2 Tregs were cultured in 50 U/mL IL-2 (versus the standard 300 U/mL IL-2). Repetitive Stimulation & Inflammatory Cytokine Tregs were exposed to three stimulations and IL-1B, IL-6, and IL-23 over the course of 25 days. B. Log2 fold change in percent of FOXP3 low HELIOS low “destabilized” cells in KO versus AAVS1-targeting sgRNAs. C. Representative flow plots of SRF and AAVS1-targeted Tregs following Repetitive Stimulation & Inflammatory Cytokine exposure.

FIGS. 33A-33B. demonstrate that FOXP3 regulator KO coupled with RNA-seq reveals broader effects on Treg state. A. Comparison of significantly differentially expressed genes from FLICR_tile2 deletion versus FOXP3 significantly differentially expressed genes. ENSG00000286181 indicates the reference FLICR gene. B. Differential expression of Treg associated genes across FOXP3 trans-regulators in resting and stimulated Tregs.

FIG. 34 provides ChIP-seq tracks of SATB1, SRF, and FOXP3 in stimulated human Tregs from one donor overlayed with the FOXP3 locus and cis-regulatory screen.

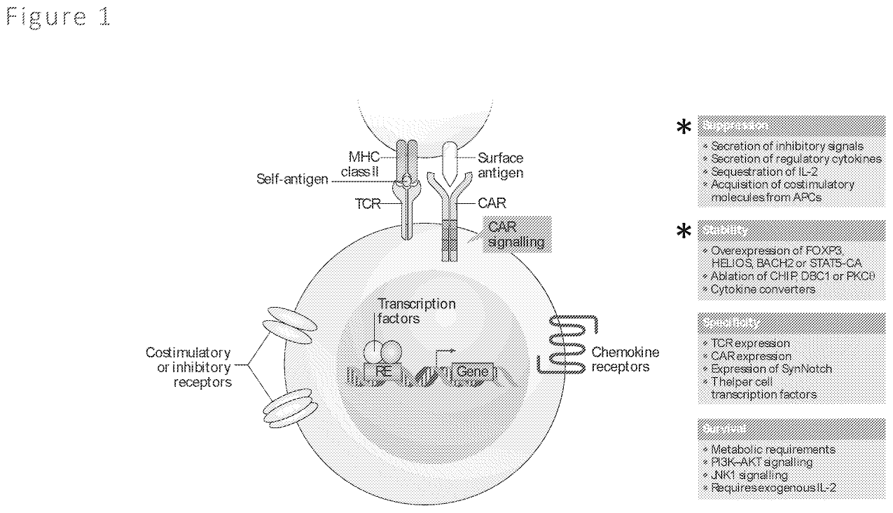

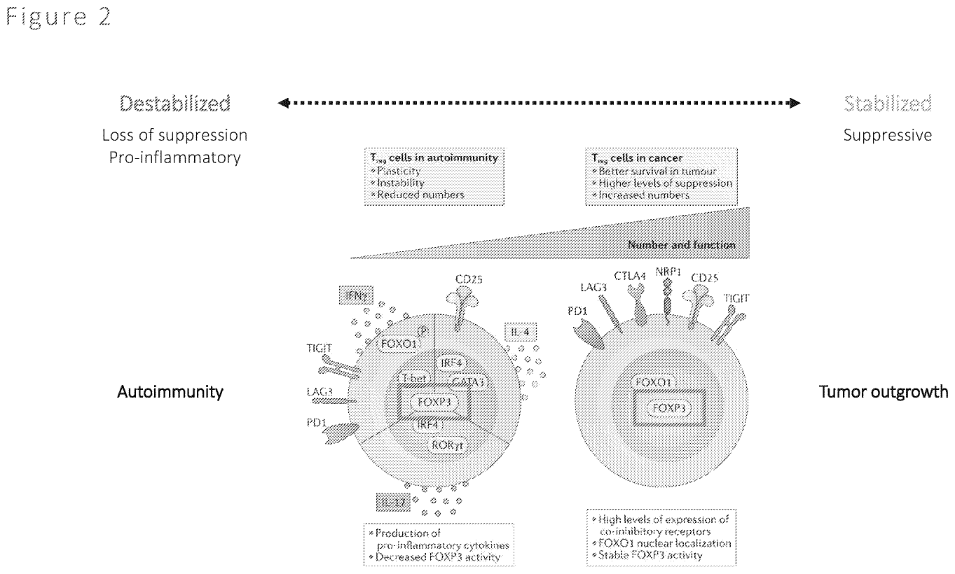

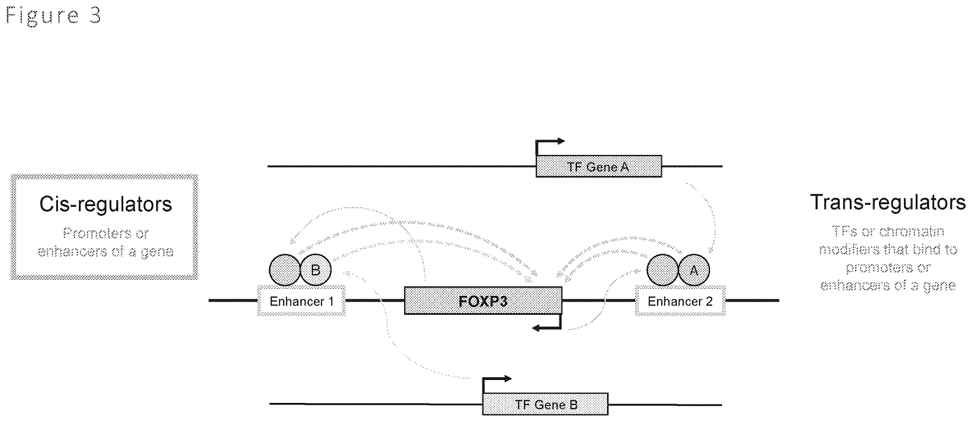

### DETAILED DESCRIPTION

Methods, kits and devices are described herein for modifying Tregs, e.g., human Tregs. The methods can include (a) incubating a sample comprising human Tregs with one or more CRISPR guide RNAs (crRNA) that bind a target site in the human genome, e.g., associated with FOXP3 expression, and a Cas enzyme or nucleic acid encoding Cas. Such methods are useful for modifying FOXP3 expression in Tregs. The resulting modified Tregs are useful in therapeutic methods.

Regulatory T cells (Tregs) are a specialized subset of CD4+ T cells that maintain self-tolerance and immune homeostasis. Due to their natural capacity to suppress inflammatory immune responses, Tregs are a promising candidate for cellular therapeutics to treat autoimmune disease, prevent transplant rejection, and heal inflamed tissues. Several dozen clinical trials of adoptive Treg cell therapy have been completed or are ongoing to treat or prevent type 1 diabetes, graft-versus-host-disease, and transplant rejection (Esensten, et al., J Allergy Clin Immun 2018). In addition, emerging and potential future applications of Treg therapies include targeting systemic lupus erythematosus, irritable bowel syndrome, autoimmune hepatitis, allergy, and asthma (Esensten, et al., J Allergy Clin Immun 2018). Critical to the safety and efficacy of Treg therapeutics is the maintenance of suppressive capacity, marked by sustained expression of the Treg lineage-defining transcription factor FOXP3 (Gavin, et al., Nature 2007). Under chronic inflammation or repeated T cell receptor stimulation, Tregs can destabilize, losing FOXP3 expression and suppressive function, and/or acquiring pro-inflammatory characteristics (Wan, et al., Nature 2007). Understanding the genetic factors controlling FOXP3 expression will nominate regulators of Treg stability and reveal manipulable targets to enhance FOXP3 expression and improve Treg cellular therapies.

To identify regulators of FOXP3, a pooled SLICE-based CRISPRn genetic screen was designed in primary human Tregs (Shifrut & Carnevale, et al., Cell 2018). A library of 6000 sgRNAs containing sgRNAs targeting 1350 human transcription factors, chromatin modifiers, and immune genes in addition to non-targeting controls was used (Freimer & Shaked, et al., Nature Genetics 2022). CD4+CD25^{high}CD127^{low} human Tregs were isolated from the blood of two healthy donors, stimulated cells with CD3/CD28/CD2 antibody complexes and delivered the sgRNA library via lentivirus and Cas9 ribonucleoproteins (RNPs) via electroporation. Cells were expanded and restimulated on day 9 post-initial stimulation. Two-days post restimulation, cells were fixed and stained for FOXP3 expression, and sorted via FACS into bins of high (top 25%) and low (bottom 25%) FOXP3 expression. Additionally, this screen in was conducted in primary human non-Treg CD4+CD25-T cells (Teffs) using similar methods as described.

31 regulators of FOXP3 expression (FDR<0.05) were identified in Tregs. Regulators included novel regulators of FOXP3 expression, in addition to previously characterized regulators, including USP22 (Cortez, et al., Nature 2020). Notably, nine FOXP3-suppressive trans-regulators were identified. Additionally, 38 regulators of FOXP3 expression (FDR<0.05) were identified in Teffs.

Ablation of these elements under destabilizing conditions in Tregs revealed a subset that prevents destabilization and enhances FOXP3 expression. Using RNA-seq coupled with CRISPR knockouts, transcriptomic changes associated with ablation of these suppressors were characterized. Knock-out of suppressors altered levels of various classic Treg-associated genes, including CTLA4 and IL2RA. Notably, knock-out of YBX1 in Tregs showed further up- and down-regulation of Treg stimulation-responsive genes, while cis-element deletion more narrowly altered gene expression changes consistent with FOXP3 up-regulation. Overall, this work reveals a network of novel cis- and trans-regulators governing FOXP3 expression and Treg stability and can be used in the rational design of next-generation Treg cellular immunotherapies.

### (CRISPR)/CRISPR-Associated (Cas) Systems

Genomic editing has been performed by using clustered regularly interspaced short palindromic repeats (CRISPR)/CRISPR-associated (Cas) systems (see e.g., Marraffini and Sontheimer. Nature Reviews Genetics 11:181-190 (2010); Sorek et al. Nature Reviews Microbiology 2008 6:181-6; Karginov and Hannon. Mol Cell 2010 1:7-19; Hale et al. Mol Cell 2010:45:292-302; Jinek et al. Science 2012 337:815-820; Bikard and Marraffini Curr Opin Immunol 2012 24:15-20; Bikard et al. Cell Host & Microbe 2012 12:177-186; all of which are incorporated by reference herein in their entireties).

However, a CRISPR guide RNA system can be adapted for use in the methods and compositions described herein. Two RNAs can be used in CRISPR genomic editing systems: a CRISPR RNA (crRNA), which is a 17-20 nucleotide sequence complementary to the target RNA, and a trans-activating crRNA (tracrRNA) that is a binding scaffold for the Cas nuclease. In some cases, the two RNAs are fused to make a single guide RNA (sgRNA). The tracrRNA forms a stem loop that is recognized and bound by the cas nuclease. The crRNA typically has shorter sequence than the tracrRNA. The term “guide RNA” as used herein refers to either a single guide RNA (sgRNA) or a crRNA. The CRISPR technique is generally described, for example, by Mali et al. Science 339:823-6 (2013); which is incorporated by reference herein in its entirety.

The guide RNA system used herein is encoded within or adjacent to the ncRNA coding region of the expression cassettes. Hence, upon transcription of the guide RNA, it can target a Cas enzyme to the desired location in the genome, where it can cleave the genomic RNA for generation of a genomic modification or alter gene expression at the site by targeting CRISPRi, CRISPRa, CRISPRon, or CRISPRoff machinery to the site.

### CRISPR/Cas

The Type II CRISPR is a well characterized system that carries out targeted DNA double-strand break in four sequential steps. First, two non-coding RNA, the pre-crRNA array and tracrRNA, are transcribed from the CRISPR locus. Second, tracrRNA hybridizes to the repeat regions of the pre-crRNA and mediates the processing of pre-crRNA into mature crRNAs containing individual spacer sequences. Third, the mature crRNA: tracrRNA complex directs Cas9 to the target DNA via Watson-Crick base-pairing between the spacer on the crRNA and the protospacer on the target DNA next to the protospacer adjacent motif (PAM), an additional requirement for target recognition. Finally, Cas9 mediates cleavage of target DNA to create a double-stranded break within the protospacer. Activity of the CRISPR/Cas system comprises of three steps: (i) insertion of alien DNA sequences into the CRISPR array to prevent future attacks, in a process called ‘adaptation,’ (ii) expression of the relevant proteins, as well as expression and processing of the array, followed by (iii) RNA-mediated interference with the alien nucleic acid. Thus, in the bacterial cell, several of the so-called ‘Cas’ proteins are involved with the natural function of the CRISPR/Cas system. The primary products of the CRISPR loci appear to be short RNAs that contain the invader targeting sequences, and are termed guide RNAs “Cas1” polypeptide refers to CRISPR associated (Cas) protein1. Cas1 (COG1518 in the Clusters of Orthologous Group of proteins classification system) is the best marker of the CRISPR-associated systems (CASS). Based on phylogenetic comparisons, seven distinct versions of the CRISPR-associated immune system have been identified (CASS1-7). Cas1 polypeptide used in the methods described herein can be any Cas1 polypeptide present in any prokaryote. In certain embodiments, a Cas1 polypeptide is a Cas1 polypeptide of an archaeal microorganism. In certain embodiments, a Cas1 polypeptide is a Cas1 polypeptide of a Euryarchaeota microorganism. In certain embodiments, a Cas1 polypeptide is a Cas1 polypeptide of a Crenarchaeota microorganism. In certain embodiments, a Cas1 polypeptide is a Cas1 polypeptide of a bacterium. In certain embodiments, a Cas1 polypeptide is a Cas1 polypeptide of a gram negative or gram-positive bacteria. In certain embodiments, a Cas1 polypeptide is a Cas1 polypeptide of Pseudomonas aeruginosa. In certain embodiments, a Cas1 polypeptide is a Cas1 polypeptide of Aquifex aeolicus. In certain embodiments, a Cas1 polypeptide is a Cas1 polypeptide that is a member of one of CASs1-7. In certain embodiments, Cas1 polypeptide is a Cas1 polypeptide that is a member of CASS3. In certain embodiments, a Cas1 polypeptide is a Cas1 polypeptide that is a member of CASS7. In certain embodiments, a Cas1 polypeptide is a Cas1 polypeptide that is a member of CASS3 or CASS7.

In some embodiments, a Cas1 polypeptide is encoded by a nucleotide sequence provided in GenBank at, e.g., GeneID number: 2781520, 1006874, 9001811, 947228, 3169280, 2650014, 1175302, 3993120, 4380485, 906625, 3165126, 905808, 1454460, 1445886, 1485099, 4274010, 888506, 3169526, 997745, 897836, or 1193018 and/or an amino acid sequence exhibiting homology (e.g., greater than 80%, 90 to 99% including 91%, 92%, 93%, 94%, 95%, 96%, 97%, 98% or 99%) to the amino acids encoded by these polynucleotides and which polypeptides function as Cas1 polypeptides.

There are three types of CRISPR/Cas systems which all incorporate RNAs and Cas proteins. Types I and III both have Cas endonucleases that process the pre-crRNAs, that, when fully processed into crRNAs, assemble a multi-Cas protein complex that is capable of cleaving nucleic acids that are complementary to the crRNA.

In type II CRISPR/Cas systems, crRNAs are produced using a different mechanism where a trans-activating RNA (tracrRNA) complementary to repeat sequences in the pre-crRNA, triggers processing by a double strand-specific RNase III in the presence of the Cas9 protein. Cas9 is then able to cleave a target DNA that is complementary to the mature crRNA however cleavage by Cas 9 is dependent both upon base-pairing between the crRNA and the target DNA, and on the presence of a short motif in the crRNA referred to as the PAM sequence (protospacer adjacent motif)). In addition, the tracrRNA must also be present as it base pairs with the crRNA at its 3′ end, and this association triggers Cas9 activity.

The Cas9 protein has at least two nuclease domains: one nuclease domain is similar to a HNH endonuclease, while the other resembles a Ruv endonuclease domain. The HNH-type domain appears to be responsible for cleaving the DNA strand that is complementary to the crRNA while the Ruv domain cleaves the non-complementary strand.

The requirement of the crRNA-tracrRNA complex can be avoided by use of an engineered “single-guide RNA” (sgRNA) that comprises the hairpin normally formed by the annealing of the crRNA and the tracrRNA (see Jinek, et al. (2012) Science 337:816 and Cong et al. (2013) Sciencexpress/10.1126/science.1231143). In S. pyrogenes, the engineered tracrRNA: crRNA fusion, or the sgRNA, guides Cas9 to cleave the target DNA when a double strand RNA: DNA heterodimer forms between the Cas associated RNAs and the target DNA. This system comprises the Cas9 protein and an engineered sgRNA

“Cas polypeptide” encompasses a full-length Cas polypeptide, an enzymatically active fragment of a Cas polypeptide, and enzymatically active derivatives of a Cas polypeptide or fragment thereof. Suitable derivatives of a Cas polypeptide or a fragment thereof include but are not limited to mutants, fusions, covalent modifications of Cas protein or a fragment thereof.

### RNA Components of CRISPR/Cas

The Cas9 related CRISPR/Cas system comprises two RNA non-coding components: tracrRNA and a pre-crRNA array containing nuclease guide sequences (spacers) interspaced by identical direct repeats (DRs). To use a CRISPR/Cas system to accomplish genome engineering, both functions of these RNAs must be present (see Cong, et al. (2013) Sciencexpress 1/10.1126/science 1231143). In some embodiments, the tracrRNA and pre-crRNAs are supplied via separate expression constructs or as separate RNAs. In other embodiments, a chimeric RNA is constructed where an engineered mature crRNA (conferring target specificity) is fused to a tracrRNA (supplying interaction with the Cas9) to create a chimeric cr-RNA-tracrRNA hybrid (also termed a single guide RNA). (see Jinek, ibid and Cong, ibid).

Chimeric or sgRNAs can be engineered to comprise a sequence complementary to any desired target. The RNAs comprise 22 bases of complementarity to a target and of the form G[n19], followed by a protospacer-adjacent motif (PAM) of the form NGG. Alternatively, sgRNAs can be designed to target any region of interest simply by identifying a suitable target sequence that conforms to the G[n20] GG formula.

A polynucleotide having one or more gRNAs can be introduced into a cell as part of a vector molecule having additional sequences such as, for example, replication origins, promoters and genes encoding antibiotic resistance. Moreover, gRNAs can be introduced as naked nucleic acid, as nucleic acid complexed with an agent such as a liposome or poloxamer, or can be delivered by viruses (e.g., adenovirus, AAV, herpesvirus, retrovirus, lentivirus and integrase defective lentivirus (IDLV)).

There are several types of CRISPR systems, some of which are summarized in the chart below.

### CRISPR System Types Overview

| System | Features | Examples |
|---|---|---|
| Type I | Multiple proteins (5-7 proteins | Staphylococcus epidermidis |
| | typical), crRNA. DNA Cleavage | (Type IA) |
| | is catalyzed by Cas3. | |
| Type II | 3-4 proteins (one protein (Cas9) | Streptococcus pyogenes |
| | has nuclease activity) two RNAs. | CRISPR/Cas9, Francisella |
| | Target DNA cleavage catalyzed | novicida U112 Cpf1 |
| | by Cas9 and RNA components. | |
| Type III | Five or six proteins required for | S. epidermidis (Type IIIA); |
| | cutting, number of required RNAs | P. furiosus; (Type IIIB). |
| | unknown but expected to be one. | |
| | Type IIIB systems have the ability | |
| | to target RNA. | |

A “guide RNA” or “gRNA” as provided herein refers to a ribonucleotide sequence capable of binding a cas nuclease, thereby forming ribonucleoprotein complex. The gRNA includes a nucleotide sequence complementary to a target site (e.g., near or at a genomic site to be edited). In some cases, the guide RNA includes one or more RNA molecules. TracrRNAs can be used to facilitate assembly of a ribonucleoprotein complex that includes the gRNA together with the tracrRNA and a cas nuclease. A complementary nucleotide sequence of the guide RNA can mediate binding of the ribonucleoprotein complex to the target site thereby providing the sequence specificity of the ribonucleoprotein complex. Thus, the guide RNA includes a sequence that is complementary to a target nucleic acid sequence such that the guide RNA binds a target nucleic acid sequence.

In some cases, the complement of the guide RNA includes a sequence having a sequence identity of about 50%, 55%, 60%, 65%, 70%, 75%, 80%, 85%, 90%, 95%, 96%, 97%, 98% or 99% to a target nucleic acid (e.g., a target viral RNA sequence). In some cases, the guide RNA includes a sequence having sequence identity of about 50%, 55%, 60%, 65%, 70%, 75%, 80%, 85%, 90%, 95%, 96%, 97%, 98% or 99% to the target nucleic acid sequence. In some cases, the guide RNA or complement thereof, includes a sequence having a sequence identity of at least about 90%, 95%, or 100% to a target sequence. In some cases, segment bound by a guide RNA within the target nucleic acid is about or at least about 10, 15, 20, 25, or more nucleotides in length.

The guide RNA is a single-stranded ribonucleic acid, although in some cases it may form some double-stranded regions by folding onto itself. In some cases, the guide RNA is about 10, 20, 30, 40, 50, 60, 70, 80, 90, 100 or more nucleic acid residues in length. In some cases, the guide RNA is from about 10 to about 30 nucleic acid residues in length. In some cases, the guide RNA is about 20 nucleic acid residues in length. For example, the length of the guide RNA can be at least about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more nucleotides or residues in length. In some cases, the guide RNA is from 5 to 50, 10 to 50, 15 to 50, 20 to 50, 25 to 50, 30 to 50, 35 to 50, 40 to 50, 45 to 50, 5 to 75, 10 to 75, 15 to 75, 20 to 75, 25 to 75, 30 to 75, 35 to 75, 40 to 75, 45 to 75, 50 to 75, 55 to 75, 60 to 75, 65 to 75, 70 to 75, 5 to 100, 10 to 100, 15 to 100, 20 to 100, 25 to 100, 30 to 100, 35 to 100, 40 to 100, 45 to 100, 50 to 100, 55 to 100, 60 to 100, 65 to 100, 70 to 100, 75 to 100, 80 to 100, 85 to 100, 90 to 100, 95 to 100, or more nucleotides or residues in length. In some cases, the guide RNA is from 10 to 15, 10 to 20, 10 to 30, 10 to 40, or 10 to 50 residues in length.

Exemplary CRISPR guide RNA (crRNA)

A CRISPR guide RNA system can be adapted for use in the methods and compositions described herein. The guide RNAs can include: a CRISPR RNA (crRNA or spacer), which is a 17-20 nucleotide sequence complementary to the target DNA, and a trans-activating crRNA (tracrRNA or stem) that is a binding scaffold for the Cas nuclease. In some cases, the two RNAs are fused to make a single guide RNA (sgRNA). The tracrRNA forms a stem loop that is recognized and bound by the Cas nuclease. The term “guide RNA” as used herein refers to either a single guide RNA (sgRNA) or a crRNA (spacer). The CRISPR technique is generally described, for example, by Mali et al. Science 339:823-6 (2013); which is incorporated by reference herein in its entirety.

In some cases, the at least one CRISPR guide RNA (crRNA) has a sequence with at least 95% sequence identity to any of SEQ ID NOs: 102-379 or 402-424. In some cases, at least one CRISPR guide RNA (crRNA) has a sequence such as any of SEQ ID NOs: 102-109, 144, 146, 148, 150, 152-155, 160-163, 168-171, 176, 178, 180, 182, 192, 194, 196, 198, 220-223, 236, 238, 240, 242, 252-255, 272-275, 280-283, 288-291, 308-315, 320, 322, 324, 326, 332-336, 338, 340, 342, 344, 346, 348, 350, 364-367 or 376-379; SEQ ID NOs: 110-113, 115-125, 127-135, 164-167, 172-175, 188-191, 200, 202-212, 214, 216, 218, 224-235, 248-251, 268-271, 292-295, 300-303, 352-359 or 368-375; or 402-413, or a nucleotide sequence with at least 80%, 82%, 84%, 85%, 87%, 89%, 90%, 92%, 94%, 95%, 97%, 98% or 99% nucleic acid sequence identity thereto, or a combination thereof. In some cases, at least one CRISPR guide RNA has a sequence such as any of SEQ ID NOs: SEQ ID NOs: 102-109, 144, 146, 148, 150, 152-155, 160-163, 168-171, 176, 178, 180, 182, 192, 194, 196, 198, 220-223, 236, 238, 240, 242, 252-255, 272-275, 280-283, 288-291, 308-315, 320, 322, 324, 326, 332-336, 338, 340, 342, 344, 346, 348, 350, 364-367 or 376-379, or a nucleotide sequence with at least 80%, 82%, 84%, 85%, 87%, 89%, 90%, 92%, 94%, 95%, 97%, 98% or 99% nucleic acid sequence identity thereto, or a combination thereof. In some cases, the at least one CRISPR guide RNA includes a targeting sequence with at least 80% sequence identity to any of SEQ ID NOs: 110-113, 115-125, 127-135, 164-167, 172-175, 188-191, 200, 202-212, 214, 216, 218, 224-235, 248-251, 268-271, 292-295, 300-303, 352-359 or 368-375. In some cases, at least one CRISPR guide RNA includes a targeting sequence such as any of SEQ ID NOs: 402-413, or a combination thereof. In some cases, the cellular sample can be incubated with one or two or more crRNAs. For example, the sample can be incubated with at least two, or at least three, or at least four, or at least five, or at least six, or at least seven, or at least eight, or at least nine, or at least ten, or more crRNAs. In some cases, the at least one crRNA has at least about 70%, at least about 75%, at least about 80%, at least about 85%, at least about 90%, at least about 95%, at least about 98%, or more sequence identity to any SEQ ID NOs: 102-379 or 402-424. In some cases, the targeting sequence in at least one crRNA has at least about 70%, about 75%, at least about 80%, at least about 85%, at least about 90%, at least about 95%, at least about 98%, or more sequence identity to any SEQ ID NO:402-407. In some cases, the targeting sequence in at least one crRNA has at least about 70%, about 75%, at least about 80%, at least about 85%, at least about 90%, at least about 95%, at least about 98%, or more sequence identity to any SEQ ID NO:408-413.

In one example, 2, 3, 4, 5, 6, 7, 8, 9 or 10 or more crRNAs specific for one or more cis-or trans-regulatory regions for FOXP3 are employed in a method to alter the activity of T cells, e.g., in a physiological sample.

If the “target” in the table is “hit_type”=“negative”, then a gRNA editing a negative regulator (via CRISPRn or CRISPRi methods) enhances FOXP3 expression.

In various examples of crRNA(s) that can be used to enhance FOXP3 expression when used with CRISPRn or CRISPRi in Tregs or Teffs, the crRNA(s) can include those with SEQ ID NOs: 110-113, 115-125, 127-135, 164-167, 172-175, 188-191, 200, 202-212, 214, 216, 218, 224-235, 248-251, 268-271, 292-295, 300-303, 352-359 or 368-375, or a nucleotide sequence with at least 80%, 82%, 84%, 85%, 87%, 89%, 90%, 92%, 94%, 95%, 97%, 98% or 99% nucleic acid sequence identity thereto, or a combination thereof. These guide RNAs can also be used suppress FOXP3 expression in CRISPRa methods.

In various examples of crRNA(s) that can be used to enhance FOXP3 expression in Tregs, the crRNA(s) can include those with SEQ ID NOs: 402-407, or a nucleotide sequence with at least 80%, 82%, 84%, 85%, 87%, 89%, 90%, 92%, 94%, 95%, 97%, 98% or 99% nucleic acid sequence identity thereto, or a combination thereof. These guide RNAs can also be used suppress FOXP3 expression in CRISPRa methods.

In various examples of crRNA(s) that can be used to suppress FOXP3 expression when used with CRISPRn or CRISPRi methods in Tregs, the crRNA(s) can include those with SEQ ID NOs: SEQ ID NOs: 102-109, 144, 146, 148, 150, 152-155, 160-163, 168-171, 176, 178, 180, 182, 192, 194, 196, 198, 220-223, 236, 238, 240, 242, 252-255, 272-275, 280-283, 288-291, 308-315, 320, 322, 324, 326, 332-336, 338, 340, 342, 344, 346, 348, 350, 364-367 or 376-379 or a nucleotide sequence with at least 80%, 82%, 84%, 85%, 87%, 89%, 90%, 92%, 94%, 95%, 97%, 98% or 99% nucleic acid sequence identity thereto, or a combination thereof. These guide RNAs can also be used enhance FOXP3 expression in CRISPRa methods.

In various examples of crRNA(s) that can be used to suppress FOXP3 expression when used with CRISPRn or CRISPRi methods in Tregs, the crRNA(s) can include those with SEQ ID NOs: 408-413, or a nucleotide sequence with at least 80%, 82%, 84%, 85%, 87%, 89%, 90%, 92%, 94%, 95%, 97%, 98% or 99% nucleic acid sequence identity thereto, or a combination thereof. These guide RNAs can also be used enhance FOXP3 expression in CRISPRa methods.

In some cases, the crRNAs can include additional sequences such as spacer sequences.

### Exemplary Cells for Modification

In one embodiment, the cells that are modified with gRNAs or the genome of which is modified to have the modified sites described herein or expresses a protein have one or more of the modifications disclosed herein, are immune cells. In one embodiment, the cells are T cells. In one embodiment, the cells are Treg cells. In one embodiment, the cells are Teff cells. In one embodiment, the cells are CD4+ cells. In one embodiment, the cells are CD8+ cells. In one embodiment, the cells are CAR-T cells. In one embodiment, the cells are naive T cells, stem cell memory cells, T_{SCM}; T Central Memory cells, T_{CM}; T effector memory cells, T_{EM}; or T effector cells, T_{EFF}. In one embodiment, the cells are Th (T helper) 1, Th2, Th9, Th17, Th22, Treg (regulatory T cells), or Tfh (follicular helper T cells). In one embodiment, the cells are regulatory T cells, NK cells, or B cells.

### Exemplary Therapeutic Uses of Modified Cells

Ex vivo or in vivo genome edited immune cells can be employed for therapeutic purposes. In addition, synthetic constructs that alter immune cell function (e.g., by incorporating or using functional proteins or protein domains that are identified by the screening method) may be employed for therapeutic purposes.

Immune cells modified as described herein may be employed in a method to prevent, inhibit or treat an autoimmune disease. In one embodiment, cells of a mammal may be obtained and modified as described herein and reintroduced to the mammal to, for example, suppress an immune function in the mammal, thereby alleviating one or more symptoms of the autoimmune disease. Autoimmune diseases within the scope of this disclosure include but are not limited to rheumatoid arthritis, Crohn's disease, multiple sclerosis, systemic lupus erythematosus (SLE), autoimmune encephalomyelitis, myasthenia gravis (MG), Hashimoto's thyroiditis, Goodpasture's syndrome, pemphigus (e.g., pemphigus vulgaris), Grave's disease, autoimmune hemolytic anemia, autoimmune thrombocytopeniaurpura, scleroderma with anti-collagen antibodies, mixed connective tissue disease, polymyositis, pernicious anemia, idiopathic Addison's disease, autoimmune-associated infertility, glomerulonephritis (e.g., crescentic glomerulonephritis, proliferative glomerulonephritis), bullous pemphigoid, Sjogren's syndrome, insulin resistance, and autoimmune diabetes mellitus (type 1 diabetes mellitus; insulin-dependent diabetes mellitus). In one embodiment, the autoimmune disease is multiple sclerosis (MS), systemic sclerosis (SSc), type 1 diabetes (TID), Grave's disease (GD), systemic lupus erythematosus (SLE), aplastic anemia (AA), or vitiligo.

Immune cells modified as described herein may be employed in a method to prevent, inhibit or treat cancer. In one embodiment, cells of a mammal may be obtained and modified as described herein and reintroduced to the mammal to, for example, augment an immune function in the mammal, thereby alleviating one or more symptoms of the cancer. Cancers within the scope of this disclosure include but are not limited to carcinomas (e.g., squamous-cell carcinomas, adenocarcinomas, hepatocellular carcinomas, and renal cell carcinomas), particularly those of the bladder, bone, bowel, breast, cervix, colon (colorectal), esophagus, head, kidney, liver (hepatocellular), lung, nasopharyngeal, neck, ovary, pancreas, prostate, and stomach; leukemias, such as acute myelogenous leukemia, acute lymphocytic leukemia, acute promyelocytic leukemia (APL), acute T-cell lymphoblastic leukemia, adult T-cell leukemia, basophilic leukemia, eosinophilic leukemia, granulocytic leukemia, hairy cell leukemia, leukopenic leukemia, lymphatic leukemia, lymphoblastic leukemia, lymphocytic leukemia, megakaryocytic leukemia, micromyeloblastic leukemia, monocytic leukemia, neutrophilic leukemia and stem cell leukemia; benign and malignant lymphomas, particularly Burkitt's lymphoma, Non-Hodgkin's lymphoma and B-cell lymphoma; benign and malignant melanomas; myeloproliferative diseases; sarcomas, particularly Ewing's sarcoma, hemangiosarcoma, Kaposi's sarcoma, liposarcoma, myosarcomas, peripheral neuroepithelioma, and synovial sarcoma; tumors of the central nervous system (e.g., gliomas, astrocytomas, oligodendrogliomas, ependymomas, glioblastomas, neuroblastomas, ganglioneuromas, gangliogliomas, medulloblastomas, pineal cell tumors, meningiomas, meningeal sarcomas, neurofibromas, and Schwannomas); germ-line tumors (e.g., bowel cancer, breast cancer, prostate cancer, cervical cancer, uterine cancer, lung cancer (e.g., small cell lung cancer, mixed small cell and non-small cell cancer, pleural mesothelioma, including metastatic pleural mesothelioma small cell lung cancer and non-small cell lung cancer), ovarian cancer, testicular cancer, thyroid cancer, astrocytoma, esophageal cancer, pancreatic cancer, stomach cancer, liver cancer, colon cancer, and melanoma; mixed types of neoplasias, particularly carcinosarcoma and Hodgkin's disease; and tumors of mixed origin, such as Wilms' tumor and teratocarcinomas, among others.

Edited Tregs may be used to alleviate symptoms and/or progression of graft-versus-host disease (GvHD) and promote transplant tolerance. This can include solid organ transplants.

### Vectors or Vehicles for Delivery

Delivery vectors or vehicles include, for example, viral vectors, microparticles, nanoparticles, liposomes and other lipid-containing complexes, and other macromolecular complexes capable of mediating delivery of nucleic acid, e.g., gRNA or encoding a polypeptide, or a protein to a host cell, e.g., a gene to provide for recombinant expression of a polypeptide encoded by the gene. Vectors or vehicles can also comprise other components or functionalities that further modulate gene delivery and/or gene expression, or that otherwise provide beneficial properties. Such other components include, for example, components that influence binding or targeting to cells (including components that mediate cell-type or tissue-specific binding); components that influence uptake of the vector by the cell; components that influence localization of the transferred gene within the cell after uptake (such as agents mediating nuclear localization); and components that influence expression of the gene. Such components also might include markers, such as detectable and/or selectable markers that can be used to detect or select for cells that have taken up and are expressing the nucleic acid delivered by the vector or have taken up protein delivered by a vehicle. Such components can be provided as a natural feature of the vector (such as the use of certain viral vectors which have components or functionalities mediating binding and uptake), or vectors can be modified to provide such functionalities. Selectable markers can be positive, negative or bifunctional. Positive selectable markers allow selection for cells carrying the marker, whereas negative selectable markers allow cells carrying the marker to be selectively eliminated. A variety of such marker genes have been described, including bifunctional (i.e., positive/negative) markers (see, e.g., WO 92/08796; and WO 94/28143). Such marker genes can provide an added measure of control that can be advantageous in gene therapy contexts. A large variety of such vectors are known in the art and are generally available.

Vectors or vehicles within the scope of the disclosure include, but are not limited to, isolated nucleic acid, e.g., plasmid-based vectors which may be extra-chromosomally maintained, and viral vectors, e.g., recombinant adenovirus, retrovirus, lentivirus, herpesvirus, poxvirus, papilloma virus, or adeno-associated virus, including viral and non-viral vectors, or proteins which are present in liposomes, e.g., neutral or cationic liposomes, such as DOSPA/DOPE, DOGS/DOPE or DMRIE/DOPE liposomes, and/or associated with other molecules such as DNA-anti-DNA antibody-cationic lipid (DOTMA/DOPE) complexes. Vectors or vehicles may be administered via any route including, but not limited to, intramuscular, buccal, rectal, intravenous or intracoronary administration, and transfer to cells may be enhanced using electroporation and/or iontophoresis. In one embodiment, vectors are locally administered.

### Retroviral Vectors

Retroviral vectors exhibit several distinctive features including their ability to stably and precisely integrate into the host genome providing long-term transgene expression. These vectors can be manipulated ex vivo to eliminate infectious gene particles to minimize the risk of systemic infection and patient-to-patient transmission. Pseudotyped retroviral vectors can alter host cell tropism.

### Lentiviruses

Lentiviruses are derived from a family of retroviruses that include human immunodeficiency virus and feline immunodeficiency virus. However, unlike retroviruses that only infect dividing cells, lentiviruses can infect both dividing and nondividing cells. Although lentiviruses have specific tropisms, pseudotyping the viral envelope with vesicular stomatitis virus yields virus with a broader range (Schnepp et al., Meth. Mol. Med., 69:427 (2002)).

### Adenoviral Vectors

Adenoviral vectors may be rendered replication-incompetent by deleting the early (EIA and E1B) genes responsible for viral gene expression from the genome and are stably maintained into the host cells in an extrachromosomal form. These vectors have the ability to transfect both replicating and nonreplicating cells and, in particular, these vectors have been shown to efficiently infect cardiac myocytes in vivo, e.g., after direction injection or perfusion. Adenoviral vectors have been shown to result in transient expression of therapeutic genes in vivo, peaking at 7 days and lasting approximately 4 weeks. The duration of transgene expression may be improved in systems utilizing neural specific promoters. In addition, adenoviral vectors can be produced at very high titers, allowing efficient gene transfer with small volumes of virus.

### Adeno-Associated Virus Vectors

Recombinant adeno-associated viruses (rAAV) are derived from nonpathogenic parvoviruses, evoke essentially no cellular immune response, and produce transgene expression lasting months in most systems. Moreover, like adenovirus, adeno-associated virus vectors also have the capability to infect replicating and nonreplicating cells and are believed to be nonpathogenic to humans.

AAV vectors include but are not limited to AAV1, AAV2, AAV5, AAV7, AAV8, AAV9 or AAVrh.10.

### Plasmid DNA Vectors

Plasmid DNA is often referred to as “naked DNA” to indicate the absence of a more elaborate packaging system. Direct injection of plasmid DNA to myocardial cells in vivo has been accomplished. Plasmid-based vectors are relatively nonimmunogenic and nonpathogenic, with the potential to stably integrate in the cellular genome, resulting in long-term gene expression in postmitotic cells in vivo. Plasmid DNA may be delivered to cells as part of a macromolecular complex, e.g., a liposome or DNA-protein complex, and delivery may be enhanced using techniques including electroporation.

### Formulations and Dosages

The modified immune cells can be formulated as pharmaceutical compositions and administered to a mammalian host, such as a human patient in a variety of forms adapted to the chosen route of administration, e.g., orally or parenterally, by intravenous, intramuscular, topical or subcutaneous routes.

In one embodiment, the immune cells may be administered by infusion or injection. Solutions of the immune cells can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion may include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it may be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active agent in the required amount in the appropriate solvent with various of the other ingredients enumerated above, as required, followed by filter sterilization. In the case of sterile powders for the preparation of sterile injectable solutions, the methods of preparation include vacuum drying and the freeze drying techniques, which yield a powder of the active ingredient plus any additional desired ingredient present in the previously sterile-filtered solutions.

Useful solid carriers may include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as antimicrobial agents can be added to optimize the properties for a given use. Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Useful dosages of the cells may be from 1×10⁴ to 1×10⁶, 1×10⁵ to 1×10⁷, 1×10⁶ to 1×10⁸, 1×10⁷ to 1×10⁹, 1×10⁸ to 1×10¹⁰, 1×10¹⁰ to 1×10¹², or 1×10¹¹ to 1×10¹⁵ cells.

The amount of for use alone or with other agents will vary with the route of administration, the nature of the condition being treated and the age and condition of the patient and will be ultimately at the discretion of the attendant physician or clinician.

### Definitions

The term “about” as used herein when referring to a measurable value such as an amount, a length, and the like, is meant to encompass variations of ±20% or ±10%, more preferably ±5%, even more preferably ±1%, and still more preferably ±0.1% from the specified value.

“Recombinant” as used herein to describe a nucleic acid molecule means a polynucleotide of genomic, cDNA, bacterial, viral, semisynthetic, or synthetic origin which, by virtue of its origin or manipulation, is not associated with all or a portion of the polynucleotide with which it is associated in nature.

The term “recombinant” as used with respect to a protein or polypeptide means a polypeptide produced by expression of a recombinant polynucleotide. In general, the polynucleotide of interest is cloned and then expressed in transformed organisms, for example, as described herein. The host organism expresses the foreign nucleic acids to produce the RNA, RT-DNA, or protein under expression conditions.

As used herein, a “cell” refers to any type of cell isolated from a prokaryotic, eukaryotic, or archaeon organism, including bacteria, archaea, fungi, protists, plants, and animals, including cells from tissues, organs, and biopsies, as well as recombinant cells, cells from cell lines cultured in vitro, and cellular fragments, cell components, or organelles comprising nucleic acids. The term also encompasses artificial cells, such as nanoparticles, liposomes, polymersomes, or microcapsules encapsulating nucleic acids. The methods described herein can be performed, for example, on a sample comprising a single cell or a population of cells. The term also includes genetically modified cells.

“Recombinant host cells,” “host cells”, “cells”, “cell lines”, “cell cultures”, and other such terms denoting microorganisms or higher eukaryotic cell lines cultured as unicellular entities refer to cells which can be, or have been, used as recipients for recombinant vector or other transferred DNA, and include the original progeny of the original cell which has been transfected.

A “coding sequence” or a sequence which “encodes” a selected polypeptide or a selected RNA, is a nucleic acid molecule which is transcribed (in the case of DNA templates) into RNA and/or translated (in the case of mRNA) into a polypeptide in vivo when placed under the control of appropriate regulatory sequences (or “control elements”). The boundaries of the coding sequence can be determined by a start codon at the 5′ (amino) terminus and a translation stop codon at the 3′ (carboxy) terminus. A coding sequence can include, but is not limited to, ncRNAs, tracrRNAs, ncRNAs modified to include heterologous sequences, cDNA from viral, prokaryotic or eukaryotic ncRNA (e.g., lncRNA), mRNA, viral or prokaryotic DNA, and even synthetic DNA sequences. A transcription termination sequence may be located 3′ to the coding sequence.

Typical “control elements,” include, but are not limited to, transcription promoters, transcription enhancer elements, transcription termination signals, polyadenylation sequences (located 3′ to the translation stop codon), sequences for optimization of initiation of translation (located 5′ to the coding sequence), and translation termination sequences.

“Operably linked” refers to an arrangement of elements wherein the components so described are configured so as to perform their usual function. Thus, a given promoter operably linked to a coding sequence is capable of effecting the expression of the coding sequence when the proper polymerases are present. The promoter need not be contiguous with the coding sequence, so long as it functions to direct the expression thereof. Thus, for example, intervening untranslated yet transcribed sequences can be present between the promoter sequence and the coding sequence, and the promoter sequence can still be considered “operably linked” to the coding sequence.

“Encoded by” refers to a nucleic acid sequence which codes for a polypeptide or RNA sequence. For example, the polypeptide sequence or a portion thereof contains an amino acid sequence of at least 3 to 5 amino acids, more preferably at least 8 to 10 amino acids, and even more preferably at least 15 to 20 amino acids from a polypeptide encoded by the nucleic acid sequence. The RNA sequence or a portion thereof contains a nucleotide sequence of at least 3 to 5 nucleotides, more preferably at least 8 to 10 nucleotides, and even more preferably at least 15 to 20 nucleotides.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein, DNA, or RNA or cause other adverse consequences. That is, a nucleic acid or peptide of this invention is purified if it is substantially free of cellular material, viral material, or culture medium when obtained from nature or when produced by recombinant DNA techniques, or free from chemical precursors or other chemicals when chemically synthesized. Purity and homogeneity are typically determined using analytical chemistry techniques, for example, polyacrylamide gel electrophoresis or high-performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

“Substantially purified” generally refers to isolation of a substance (nucleic acid, compound, polynucleotide, protein, polypeptide, peptide composition) such that the substance comprises the majority percent of the sample in which it resides. Typically, in a sample, a substantially purified component comprises 50%, including 80%-85%, including 90-95% of the sample. Techniques for purifying polynucleotides and polypeptides of interest are well-known in the art and include, for example, ion-exchange chromatography, affinity chromatography and sedimentation according to density.

A “vector” is capable of transferring nucleic acid sequences to target cells (e.g., viral vectors, non-viral vectors, particulate carriers, and liposomes). Typically, “vector construct,” “expression vector,” and “gene transfer vector,” mean any nucleic acid construct capable of directing the expression of a nucleic acid of interest and which can transfer nucleic acid sequences to target cells. Thus, the term includes cloning and expression vehicles, as well as viral vectors.

“Expression” refers to detectable production of a gene product by a cell. The gene product may be a transcription product (i.e., RNA), which may be referred to as “gene expression”, or the gene product may be a translation product of the transcription product (i.e., a protein), depending on the context.

“Mammalian cell” refers to any cell derived from a mammalian subject suitable for transfection with vector systems comprising, as described herein. The cell may be xenogeneic, autologous, or allogeneic. The cell can be a primary cell obtained directly from a mammalian subject. The cell may also be a cell derived from the culture and expansion of a cell obtained from a mammalian subject. Immortalized cells are also included within this definition. In some embodiments, the cell has been genetically engineered to express a recombinant protein and/or nucleic acid.

The term “subject” includes animals, including both vertebrates and invertebrates, including, without limitation, invertebrates such as arthropods, mollusks, annelids, and cnidarians; and vertebrates such as amphibians, including frogs, salamanders, and caecillians; reptiles, including lizards, snakes, turtles, crocodiles, and alligators; fish; mammals, including human and non-human mammals such as non-human primates, including chimpanzees and other apes and monkey species; laboratory animals such as mice, rats, rabbits, hamsters, guinea pigs, and chinchillas; domestic animals such as dogs and cats; farm animals such as sheep, goats, pigs, horses and cows; and birds such as domestic, wild and game birds, including chickens, turkeys and other gallinaceous birds, ducks, geese, and the like. In some cases, the disclosed methods find use in experimental animals, in veterinary application, and in the development of animal models for disease, including, but not limited to, rodents including mice, rats, and hamsters; primates, and transgenic animals.

The regulatory T cells (Tregs cells; naturally occurring, peripherally induced, or induced in vitro) are a subpopulation of T cells that modulate the immune system, maintain tolerance to self-antigens, and prevent autoimmune disease. Treg cells are immunosuppressive and generally suppress or downregulate induction and proliferation of effector T cells. Treg cells express the biomarkers CD4, FOXP3, and CD25 and are thought to be derived from the same lineage as naïve CD4+ cells. T regulatory cells are a component of the immune system that suppress immune responses of other cells. This is a “self-check” built into the immune system to prevent excessive reactions. Regulatory T cells come in many forms with the most well-understood being those that express CD4, CD25, and FOXP3 (CD4+CD25+ regulatory T cells). These Treg cells are different from helper T cells. Regulatory T cells are involved in shutting down immune responses after they have successfully eliminated invading organisms, and also in preventing autoimmunity.

FOXP3 (forkhead box P3; FOX proteins belong to the forkhead/winged-helix family of transcriptional regulators and are presumed to exert control via similar DNA binding interactions during transcription), also known as scurfin, is a protein involved in immune system responses. A member of the FOX protein family, FOXP3 appears to function as a master regulator of the regulatory pathway in the development and function of regulatory T cells. Regulatory T cells generally turn the immune response down. In cancer, an excess of regulatory T cell activity can prevent the immune system from destroying cancer cells. In autoimmune disease, a deficiency of regulatory T cell activity can allow other autoimmune cells to attack the body's own tissues.

The human FOXP3 genes contain 11 coding exons. Exon-intron boundaries are identical across the coding regions of the mouse and human genes. By genomic sequence analysis, the FOXP3 gene maps to the p arm of the X chromosome (specifically, Xp11.23; Chr X: 49.25-49.27). Human mRNA sequence for FOXP3 can be found at accession numbers NM_001114377, NM_014009′ human protein sequence can be found at NP_001107849, NP_054728 (all accession numbers and their sequences are incorporated herein by references).

An example of human FOXP3 mRNA sequence (SEQ ID NO: 450)

| | |
|---|---|
| 1 | agtttcccac aagccaggct gatccttttc tgtcagtcca cttcaccaag cctgcccttg |
| 61 | gacaaggacc cgatgcccaa ccccaggcct ggcaagccct cggccccttc cttggccctt |
| 121 | ggcccatccc caggagcctc gcccagctgg agggctgcac ccaaagcctc agacctgctg |
| 181 | ggggcccggg gcccaggggg aaccttccag ggccgagatc ttcgaggcgg ggcccatgcc |
| 241 | tcctcttctt ccttgaaccc catgccacca tcgcagctgc agctctcaac ggtggatgcc |
| 301 | cacgcccgga cccctgtgct gcaggtgcac cccctggaga gcccagccat gatcagcctc |
| 361 | acaccaccca ccaccgccac tggggtcttc tccctcaagg cccggcctgg cctcccacct |
| 421 | gggatcaacg tggccagcct ggaatgggtg tccagggagc cggcactgct ctgcaccttc |
| 481 | ccaaatccca gtgcacccag gaaggacagc accctttcgg ctgtgcccca gagctcctac |
| 541 | ccactgctgg caaatggtgt ctgcaagtgg cccggatgtg agaaggtctt cgaagagcca |
| 601 | gaggacttcc tcaagcactg ccaggcggac catcttctgg atgagaaggg cagggcacaa |
| 661 | tgtctcctcc agagagagat ggtacagtct ctggagcagc agctggtgct ggagaaggag |
| 721 | aagctgagtg ccatgcaggc ccacctggct gggaaaatgg cactgaccaa ggcttcatct |
| 781 | gtggcatcat ccgacaaggg ctcctgctgc atcgtagctg ctggcagcca aggccctgtc |
| 841 | gtcccagcct ggtctggccc ccgggaggcc cctgacagcc tgtttgctgt ccggaggcac |
| 901 | ctgtggggta gccatggaaa cagcacattc ccagagttcc tccacaacat ggactacttc |
| 961 | aagttccaca acatgcgacc ccctttcacc tacgccacgc tcatccgctg ggccatcctg |
| 1021 | gaggctccag agaagcagcg gacactcaat gagatctacc actggttcac acgcatgttt |
| 1081 | gccttcttca gaaaccatcc tgccacctgg aagaacgcca tccgccacaa cctgagtctg |
| 1141 | cacaagtgct ttgtgcgggt ggagagcgag aagggggctg tgtggaccgt ggatgagctg |
| 1201 | gagttccgca agaaacggag ccagaggccc agcaggtgtt ccaaccctac acctggcccc |
| 1261 | tgacctcaag atcaaggaaa ggaggatgga cgaacagggg ccaaactggt gggaggcaga |
| 1321 | ggtggtgggg gcagggatga taggccctgg atgtgcccac agggaccaag aagtgaggtt |
| 1381 | tccactgtct tgcctgccag ggcccctgtt cccccgctgg cagccacccc ctcccccatc |
| 1441 | atatcctttg ccccaaggct gctcagaggg gccccggtcc tggccccagc ccccacctcc |
| 1501 | gccccagaca caccccccag togagccctg cagccaaaca gagccttcac aaccagccac |
| 1561 | acagagcctg cctcagctgc tcgcacagat tacttcaggg ctggaaaagt cacacagaca |
| 1621 | cacaaaatgt cacaatcctg tccctcactc aacacaaacc ccaaaacaca gagagcctgc |
| 1681 | ctcagtacac tcaaacaacc tcaaagctgc atcatcacac aatcacacac aagcacagcc |
| 1741 | ctgacaaccc acacacccca aggcacgcac ccacagccag cctcagggcc cacaggggca |
| 1801 | ctgtcaacac aggggtgtgc ccagaggcct acacagaagc agcgtcagta ccctcaggat |
| 1861 | ctgaggtccc aacacgtgct cgctcacaca cacggcctgt tagaattcac ctgtgtatct |
| 1921 | cacgcatatg cacacgcaca gccccccagt gggtctcttg agtcccgtgc agacacacac |
| 1981 | agccacacac actgccttgc caaaaatacc ccgtgtctcc cctgccactc acctcactcc |
| 2041 | cattccctga gccctgatcc atgcctcagc ttagactgca gaggaactac tcatttattt |
| 2101 | gggatccaag gcccccaacc cacagtaccg tccccaataa actgcagccg agctcccca |

An example of human FOXP3 protein sequence (SEQ ID NO: 451)

| | |
|---|---|
| 1 | MPNPRPGKPS APSLALGPSP GASPSWRAAP KASDLLGARG PGGTFQGRDL RGGAHASSSS |
| 61 | LNPMPPSQLQ LPTLPLVMVA PSGARLGPLP HLQALLQDRP HFMHQLSTVD AHARTPVLQV |
| 121 | HPLESPAMIS LTPPTTATGV FSLKARPGLP PGINVASLEW VSREPALLCT FPNPSAPRKD |
| 181 | STLSAVPQSS YPLLANGVCK WPGCEKVFEE PEDFLKHCQA DHLLDEKGRA QCLLQREMVQ |
| 241 | SLEQQLVLEK EKLSAMQAHL AGKMALTKAS SVASSDKGSC CIVAAGSQGP VVPAWSGPRE |
| 301 | APDSLFAVRR HLWGSHGNST FPEFLHNMDY FKFHNMRPPF TYATLIRWAI LEAPEKQRTL |
| 361 | NEIYHWFTRM FAFFRNHPAT WKNAIRHNLS LHKCFVRVES EKGAVWTVDE LEFRKKRSQR |
| 421 | PSRCSNPTPG P |

In regulatory T cell model systems, the FOXP3 transcription factor occupies the promoters for genes involved in regulatory T-cell function and may inhibit transcription of key genes following stimulation of T cell receptors.

“Gene transfer” or “gene delivery” refers to methods or systems for reliably inserting DNA or RNA of interest into a host cell. Such methods can result in transient expression of non-integrated transferred DNA, extrachromosomal replication and expression of transferred replicons (e.g., episomes), or integration of transferred genetic material into the genomic DNA of host cells. Gene delivery expression vectors include, but are not limited to, vectors derived from bacterial plasmid vectors, viral vectors, non-viral vectors, alphaviruses, pox viruses and vaccinia viruses.

The term “derived from” is used herein to identify the original source of a molecule but is not meant to limit the method by which the molecule is made which can be, for example, by chemical synthesis or recombinant means.

A polynucleotide or nucleic acid “derived from” a designated sequence refers to a polynucleotide or nucleic acid that includes a contiguous sequence of approximately at least about 6 nucleotides, preferably at least about 8 nucleotides, more preferably at least about 10-12 nucleotides, and even more preferably at least about 15-20 nucleotides corresponding, i.e., identical or complementary to, a region of the designated nucleotide sequence. The derived polynucleotide will not necessarily be derived physically from the nucleotide sequence of interest, but may be generated in any manner, including, but not limited to, chemical synthesis, replication, reverse transcription or transcription, which is based on the information provided by the sequence of bases in the region(s) from which the polynucleotide is derived. As such, it may represent either a sense or an antisense orientation of the original polynucleotide.

The terms “hybridize” and “hybridization” refer to the formation of complexes between nucleotide sequences which are sufficiently complementary to form complexes via Watson-Crick base pairing.

The term “homologous region” refers to a region of a nucleic acid with homology to another nucleic acid region. Thus, whether a “homologous region” is present in a nucleic acid molecule is determined with reference to another nucleic acid region in the same or a different molecule. Further, since a nucleic acid is often double-stranded, the term “homologous, region,” as used herein, refers to the ability of nucleic acid molecules to hybridize to each other. For example, a single-stranded nucleic acid molecule can have two homologous regions which are capable of hybridizing to each other. Thus, the term “homologous region” includes nucleic acid segments with complementary sequences. Homologous regions may vary in length but will typically be between 4 and 500 nucleotides (e.g., from about 4 to about 40, from about 40 to about 80, from about 80 to about 120, from about 120 to about 160, from about 160 to about 200, from about 200 to about 240, from about 240 to about 280, from about 280 to about 320, from about 320 to about 360, from about 360 to about 400, from about 400 to about 440, etc.).

As used herein, the terms “complementary” or “complementarity” refers to polynucleotides that are able to form base pairs with one another. Base pairs are typically formed by hydrogen bonds between nucleotide units in an anti-parallel orientation between polynucleotide strands. Complementary polynucleotide strands can base pair in a Watson-Crick manner (e.g., A to T, A to U, C to G), or in any other manner that allows for the formation of duplexes. As persons skilled in the art are aware, when using RNA as opposed to DNA, uracil (U) rather than thymine (T) is the base that is considered to be complementary to adenosine. However, when uracil is denoted in the context of the present invention, the ability to substitute a thymine is implied, unless otherwise stated. “Complementarity” may exist between two RNA strands, two DNA strands, or between an RNA strand and a DNA strand. It is generally understood that two or more polynucleotides may be “complementary” and able to form a duplex despite having less than perfect or less than 100% complementarity. Two sequences are “perfectly complementary” or “100% complementary” if at least a contiguous portion of each polynucleotide sequence, comprising a region of complementarity, perfectly base pairs with the other polynucleotide without any mismatches or interruptions within such region. Two or more sequences are considered “perfectly complementary” or “100% complementary” even if either or both polynucleotides contain additional non-complementary sequences as long as the contiguous region of complementarity within each polynucleotide is able to perfectly hybridize with the other. “Less than perfect” complementarity refers to situations where less than all of the contiguous nucleotides within such region of complementarity are able to base pair with each other. Determining the percentage of complementarity between two polynucleotide sequences is a matter of ordinary skill in the art.

The term “donor polynucleotide” or “donor DNA” refers to a nucleic acid or polynucleotide that provides a nucleotide sequence of an intended edit to be integrated into the genome at a target locus by HDR or recombineering.

A “target site” or “target sequence” is the nucleic acid sequence recognized (i.e., sufficiently complementary for hybridization) by a guide RNA (gRNA) or a homology arm of a donor polynucleotide (donor DNA). The target site may be allele-specific (e.g., a major or minor allele). For example, a target site can be a genomic site that is intended to be modified such as by insertion of one or more nucleotides, replacement of one or more nucleotides, deletion of one or more nucleotides, or a combination thereof.

In general, “a CRISPR system” refers collectively to transcripts and other elements involved in the expression of or directing the activity of CRISPR-associated (“Cas”) genes, including sequences encoding a Cas gene, and a CRISPR array nucleic acid sequence including a leader sequence and at least one repeat sequence. In some embodiments, one or more elements of a CRISPR system are derived from a type I, type II, or type III CRISPR system. Cas1 and Cas2 are found in all three types of CRISPR-Cas systems, and they are involved in spacer acquisition. In the I-E system of E. coli, Cas1 and Cas2 form a complex where a Cas2 dimer bridges two Cas1 dimers. In this complex Cas2 performs a non-enzymatic scaffolding role, binding double-stranded fragments of invading DNA, while Cas1 binds the single-stranded flanks of the DNA and catalyzes their integration into CRISPR arrays.

In some embodiments, one or more elements of a CRISPR system are derived from a particular organism comprising an endogenous CRISPR system, such as Streptococcus pyogenes. In general, a CRISPR system can be characterized by elements that promote the formation of a CRISPR complex at the site of a target sequence (also referred to as a protospacer in the context of an endogenous CRISPR system).

In certain embodiments, the disclosure provides protospacers that are adjacent to short (3-5 bp) DNA sequences termed protospacer adjacent motifs (PAM). The PAMs are important for type I and type II systems during acquisition. In type I and type II systems, protospacers are excised at positions adjacent to a PAM sequence, with the other end of the spacer cut using a ruler mechanism, thus maintaining the regularity of the spacer size in the CRISPR array. The conservation of the PAM sequence differs between CRISPR-Cas systems and may be evolutionarily linked to Cas1 and the leader sequence.

In some embodiments, a regulatory element is operably linked to one or more elements of a CRISPR system so as to drive expression of the one or more elements of the CRISPR system. In general, CRISPRs (Clustered Regularly Interspaced Short Palindromic Repeats), also known as SPIDRs (SPacer Interspersed Direct Repeats), constitute a family of DNA loci that are usually specific to a particular bacterial species. The CRISPR locus comprises a distinct class of interspersed short sequence repeats (SSRs) that were recognized in E. coli (Ishino et al, J. BacterioL, 169:5429-5433 (1987); and Nakata et al., J. BacterioL, 171:3553-3556 (1989)), and associated genes. Similar interspersed SSRs have been identified in Haloferax mediterranei, Streptococcus pyogenes, Anabaena, and Mycobacterium tuberculosis (See, Groenen et al., Mol. Microbiol., 10:1057-1065 (1993); Hoe et al., Emerg. Infect. Dis., 5:254-263 (1999); Masepohl et al, Biochim. Biophys. Acta 1307:26-30 (1996); and Mojica et al, Mol. Microbiol, 17:85-93 (1995)). The CRISPR loci typically differ from other SSRs by the structure of the repeats, which have been termed short regularly spaced repeats (SRSRs) (Janssen et al, OMICS J. Integ. Biol., 6:23-33 (2002); and Mojica et al, Mol. Microbiol., 36:244-246 (2000)). In general, the repeats are short elements that occur in clusters that are regularly spaced by unique intervening sequences with a substantially constant length (Mojica et al., (2000), supra). Although the repeat sequences are highly conserved between strains, the number of interspersed repeats and the sequences of the spacer regions typically differ from strain to strain (van Embden et al., J. Bacteriol., 182:2393-2401 (2000)). CRISPR loci have been identified in more than 40 prokaryotes (See e.g., Jansen et al, Mol. Microbiol., 43:1565-1575 (2002); and Mojica et al, (2005)) including, but not limited to Aeropyrum, Pyrobaculum, Sulfolobus, Archaeoglobus, Halocarcula, Methanobacteriumn, Methanococcus, Methanosarcina, Methanopyrus, Pyrococcus, Picrophilus, Thernioplasnia, Corynebacterium, Mycobacterium, Streptomyces, Aquifrx, Porphvromonas, Chlorobium, Thermus, Bacillus, Listeria, Staphylococcus, Clostridium, Thermoanaerobacter, Mycoplasma, Fusobacterium, Azarcus, Chromobacterium, Neisseria, Nitrosomonas, Desulfovibrio, Geobacter, Myrococcus, Campylobacter, Wolinella, Acinetobacter, Erwinia, Escherichia, Legionella, Methylococcus, Pasteurella, Photobacterium, Salmonella, Xanthomonas, Yersinia, Treponema, and Thermotoga.

In some embodiments, an enzyme coding sequence encoding a CRISPR enzyme (e.g., cas9) is codon optimized for expression in particular cells, such as eukaryotic cells. The eukaryotic cells may be those of or derived from a particular organism, such as a mammal, including but not limited to human, mouse, rat, rabbit, dog, or non-human primate. In general, codon optimization refers to a process of modifying a nucleic acid sequence for enhanced expression in the host cells of interest by replacing at least one codon (e.g. about one or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database”, and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, Pa.), are also available. In some embodiments, one or more codons (e.g., 1, 2, 3, 4, 5, 10, 15, 20, 25, 50, or more, or all codons) in a sequence encoding a CRISPR enzyme correspond to the most frequently used codon for a particular amino acid.

“Administering” a nucleic acid, such as an expression cassette, comprises transducing, transfecting, electroporating, translocating, fusing, phagocytosing, shooting or ballistic methods, etc., i.e., any means by which a nucleic acid can be transported across a cell membrane.

The subject matter disclosed herein is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosed subject matter. The upper and lower limits of these smaller ranges may independently be included in the smaller ranges, and are also encompassed within the disclosed subject matter, subject to any specifically excluded limit in the stated range. Where the stated range includes one or both of the limits, ranges excluding either or both of those included limits are also included in the disclosed subject matter.

Unless defined otherwise, all technical and scientific terms used herein have the same meaning as commonly understood by one of ordinary skill in the art to which the disclosed subject matter belongs. Although any methods and materials similar or equivalent to those described herein can also be used in the practice or testing of the disclosed subject matter, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a cell” includes a plurality of such cells and reference to “the nucleic acid” includes reference to one or more nucleic acids and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of any features or elements described herein, which includes use of a “negative” limitation.

It is appreciated that certain features of the disclosed subject matter, which are, for clarity, described in the context of separate embodiments, may also be provided in combination in a single embodiment. Conversely, various features of the disclosed subject matter, which are, for brevity, described in the context of a single embodiment, may also be provided separately or in any suitable sub-combination. All combinations of the embodiments pertaining to the disclosure are specifically embraced by the disclosed subject matter and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

The publications discussed herein are provided solely for their disclosure prior to the filing date of the present application. Nothing herein is to be construed as an admission that the disclosed subject matter is not entitled to antedate such publication. Further, the dates of publication provided may be different from the actual publication dates which may need to be independently confirmed.

The following Examples illustrate some of the materials, methods, and experiments that were used or performed in the development of the invention.

### EXAMPLES

Tregs are suppressive immune cells, but under certain environmental conditions, they can lose this capacity and even acquire pro-inflammatory capacity, which cells are referred to as destabilized Tregs. CRISPR technologies may be employed to dissect genetic networks controlling FOXP3 expression in primary human Tregs. As disclosed herein below, cis-regulatory elements of FOXP3 expression in Treg and Teff cells were identified (shared and specific elements), as well as trans-regulators of FOXP3 expression in Treg and Teff cells (shared and specific elements). The role of trans- and cis-regulators of FOXP3 under stability challenge, e.g., inflammatory cytokines, low IL-2 or antigenic ignorance, may be determined.

### Example 1

Methods of boosting (or inhibiting) FOXP3 expression in Tregs can be employed to engineer improved cell therapies. CRISPR interference (CRISPRi) and CRISPR nuclease (CRISPRn) genetic screens in human regulatory T cells (Tregs) and non-Treg CD4+ T cells (Teff) were used to identify cis- and trans-regulators of FOXP3 expression. By tiling CRISPRi machinery across approximately 95 kb of the FOXP 3 locus, positive and negative cis regulators of FOXP3 expression were identified. A noncoding region upstream of the FOXP3 promoter involved in FOXP3 suppression was one of those identified sites. This region, referred to as FLICR, maps to the human homologue of Flicr, a lncRNA previously described in murine Tregs (Zemmour, D. et al. PNAS 2017). Specific guide RNAs targeting this locus were identified that can be introduced into human Tregs to increase FOXP3 expression. Subsequent validation of cis-regulators using arrayed CRISPRn deletion of elements confirmed the FOXP3-suppressive capacity of FLICR.

Additionally, CRISPRn screens targeting 1350 human transcription factors, chromatin modifiers, and immune genes yielded significant positive and negative trans-regulators of FOXP3 expression in Tregs and Teff. To assess the capacity of these cis- and trans-regulators to maintain Treg identity under strong inflammation, the regulators were knocked out (KO) or deleted in human Tregs and those cells exposed to repetitive CD28/CD3/CD2 stimulation and inflammatory cytokines. KO or deletion of multiple regulators involved in FOXP3 suppression (including, but not limited to, SRF, VARS2, and TFDP1) increased the percentage of Tregs maintaining FOXP3 and HELIOS expression and decreased the percentage losing FOXP3 and HELIOS expression relative to AAVS1-targeted controls. Methods that could improve Treg function and stability in therapies for autoimmunity or to selectively destabilize Tregs in therapies for cancer or infectious diseases are disclosed herein.

Based on the identification of positive and negative cis- and trans-regulators of FOXP3 expression and Treg identity, selective perturbation of cis- and trans-regulators in Tregs may be used to (1) improve Treg stability, and potentially function, for autoimmune treatment applications, or (2) selectively destabilize Tregs in a cancer setting to enable enhanced immune recognition of tumors. Genetic editing of those regulators in Treg cellular immunotherapies may give rise to Treg therapeutics with enhanced stability, and therefore safety and function, in autoimmune and transplant tolerance settings. Additionally, genetic editing or small-molecule targeting of the regulators in human Tregs in a cancer setting could induce selective functional inactivity of tumor-associated Tregs to enable enhanced immune recognition and clearance of tumors.

### Example 2

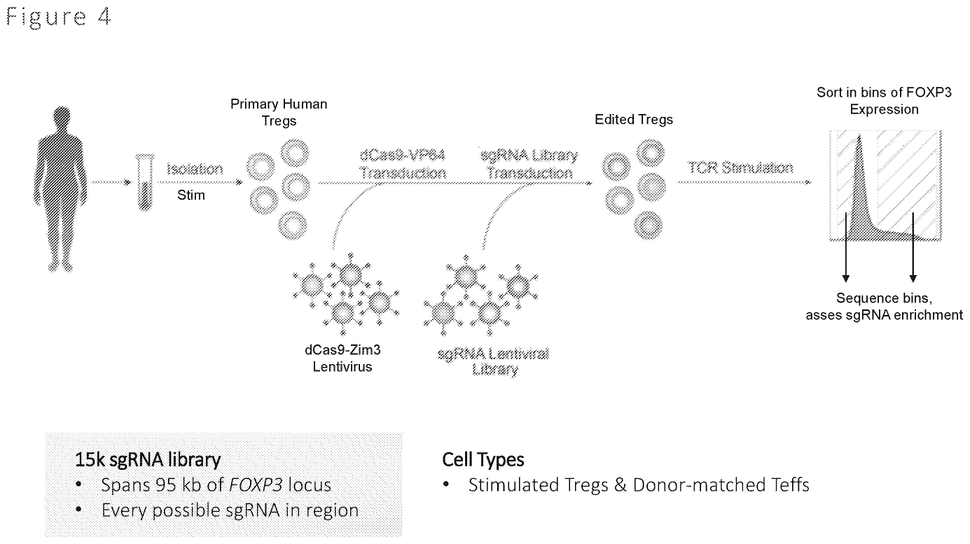

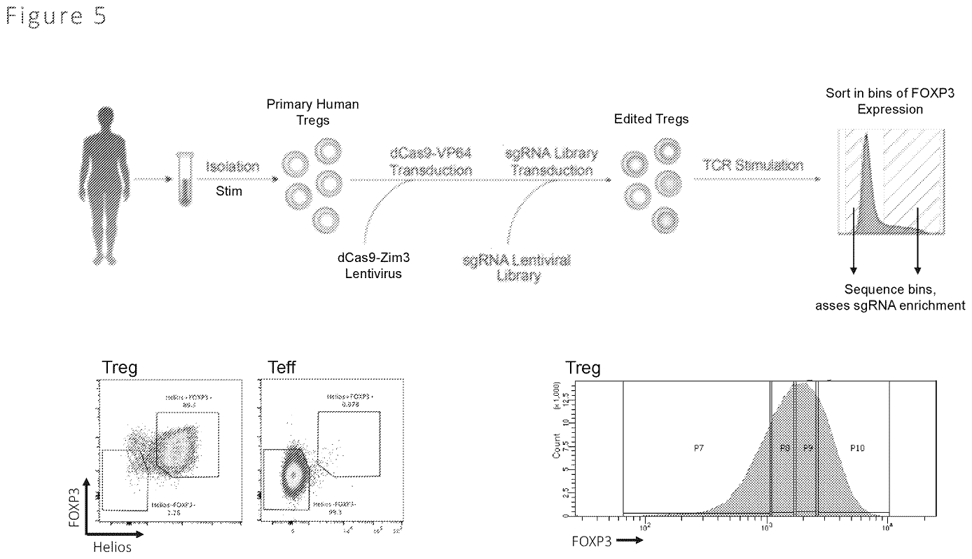

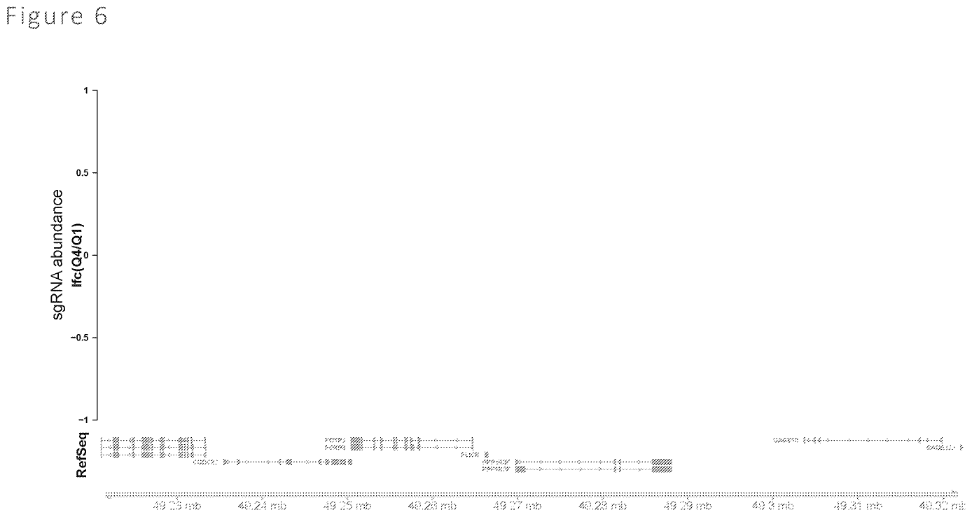

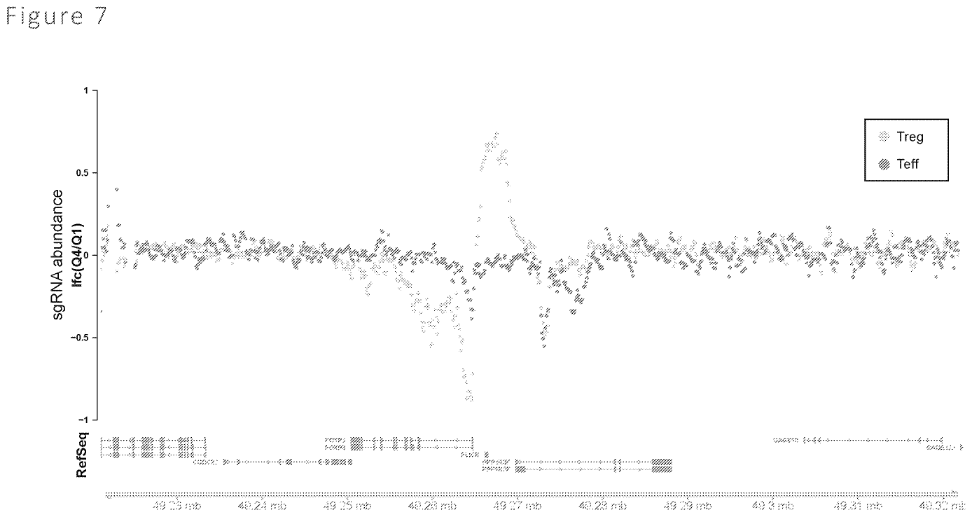

Tregs are suppressive immune cells, but under certain environmental conditions, they can lose this capacity and even acquire pro-inflammatory capacity, and we refer to this as destabilized Tregs. CRISPR technologies can dissect genetic networks controlling FOXP3 expression in primary human Tregs. To identify FOXP3 cis regulators a CRISPR interference screen was employed with a library of 15,000 gRNAs spanning 95 kb of the FOXP3 gene (FIGS. 4-7). CRISPRi tiling screen identifies FOXP3 cis-regulatory elements across the FOXP3 locus.

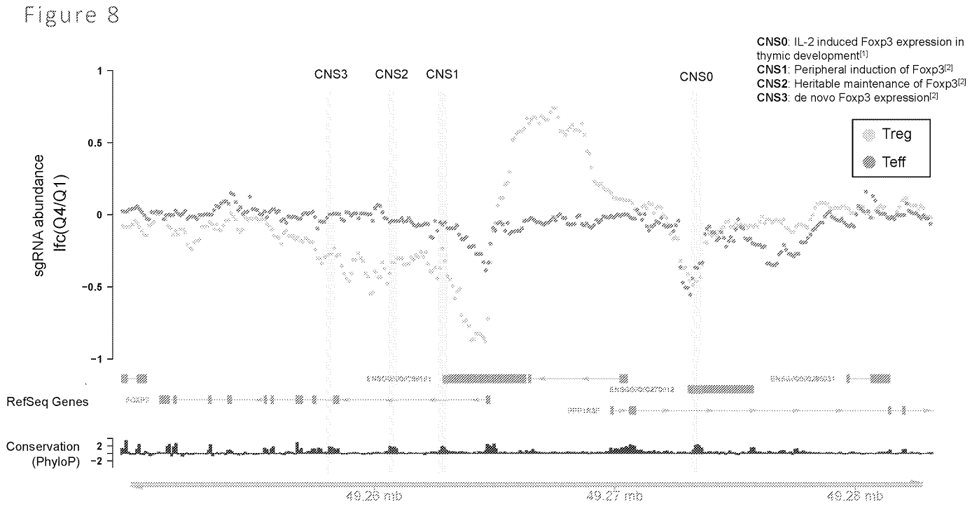

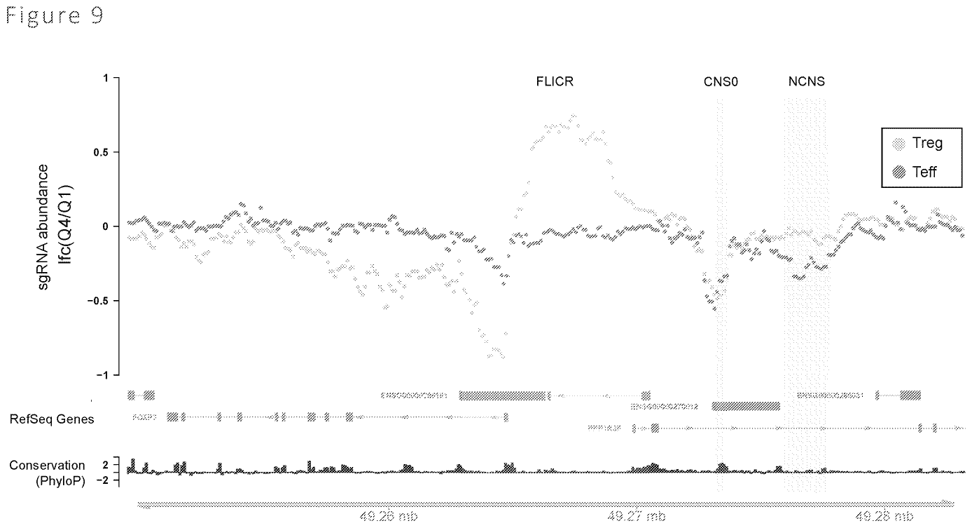

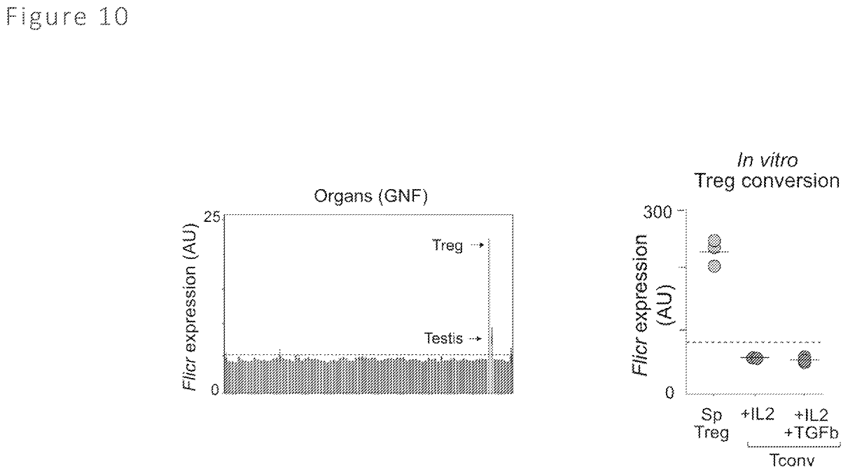

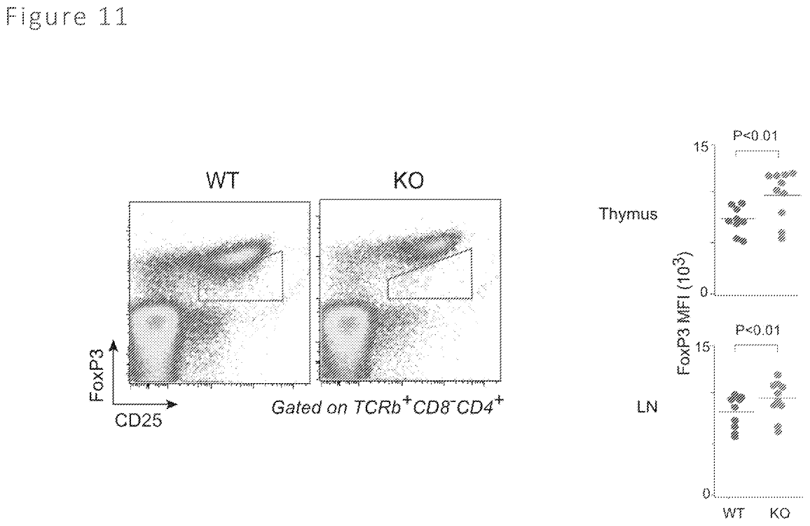

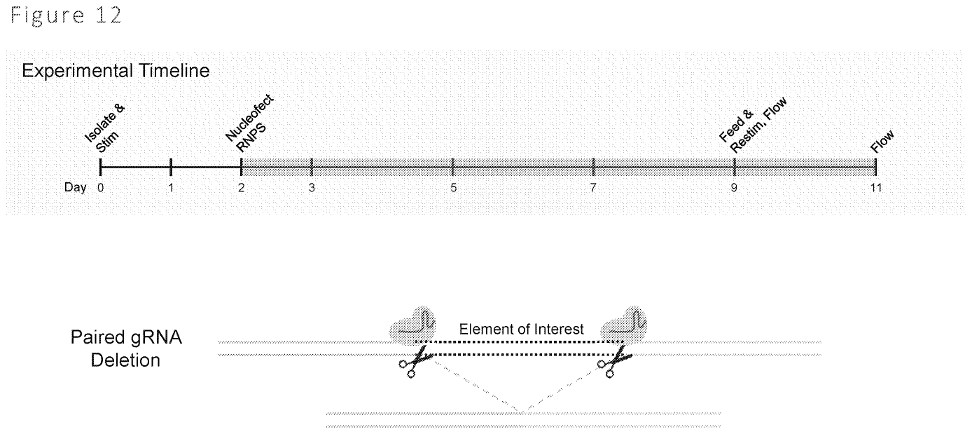

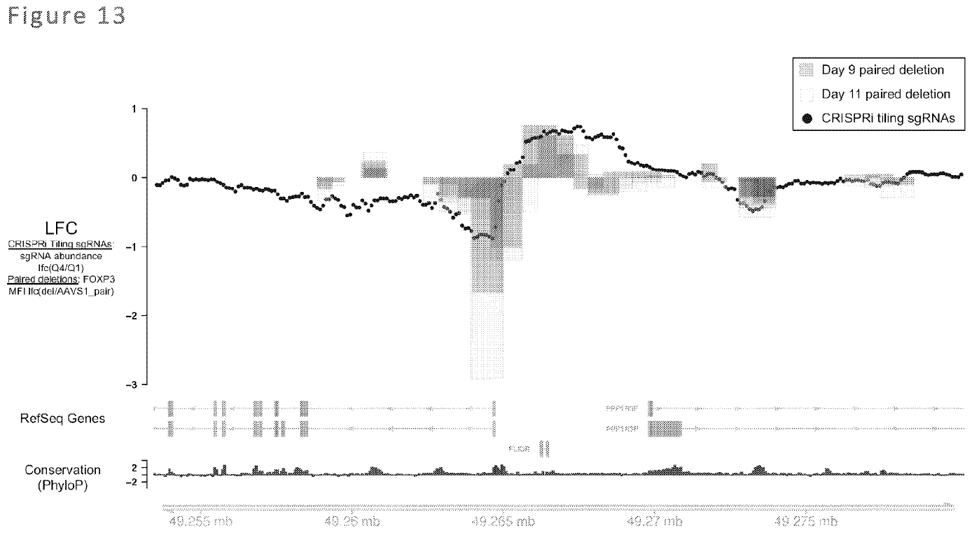

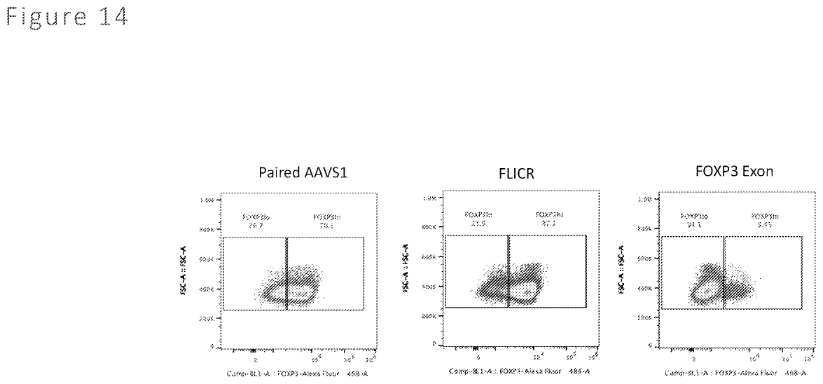

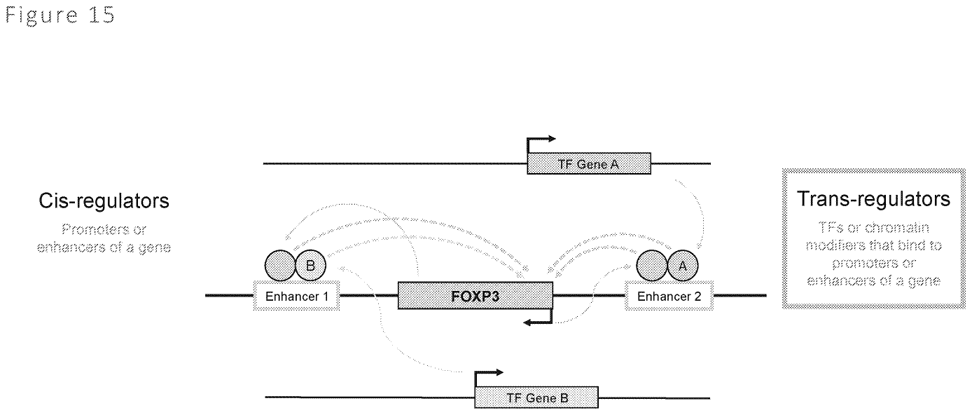

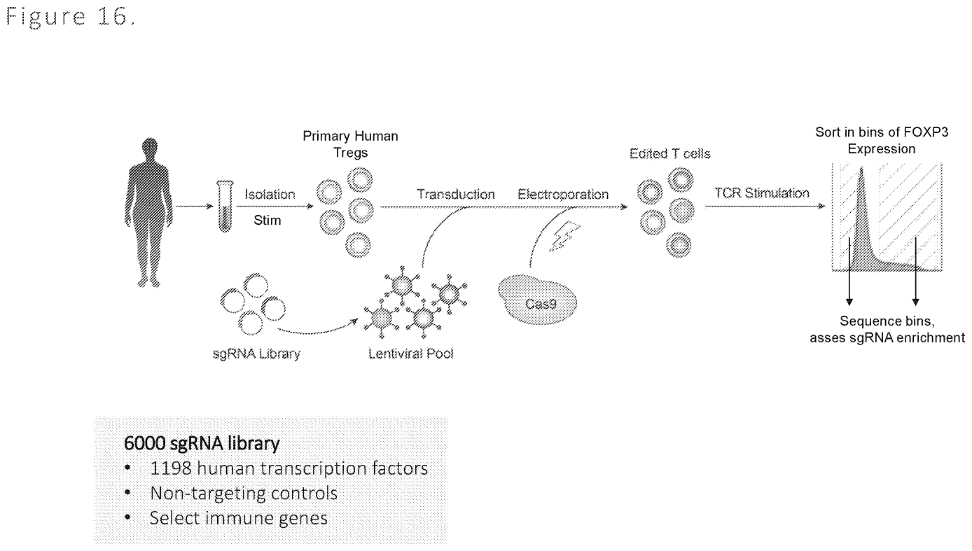

FIG. 8 expands on CNSs that were identified as maintenance cis regulators of FOXP3.

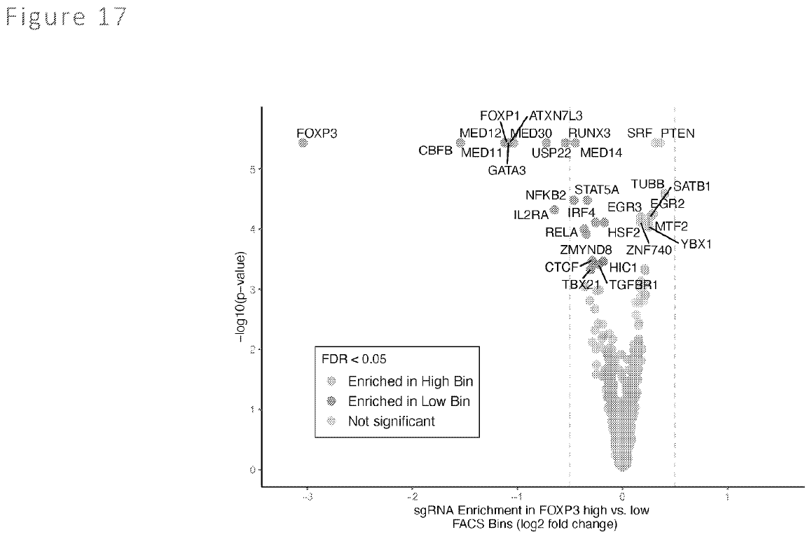

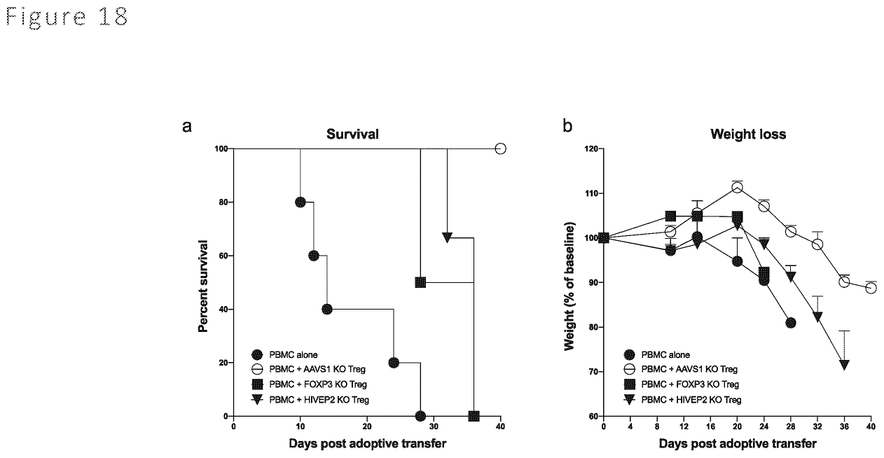

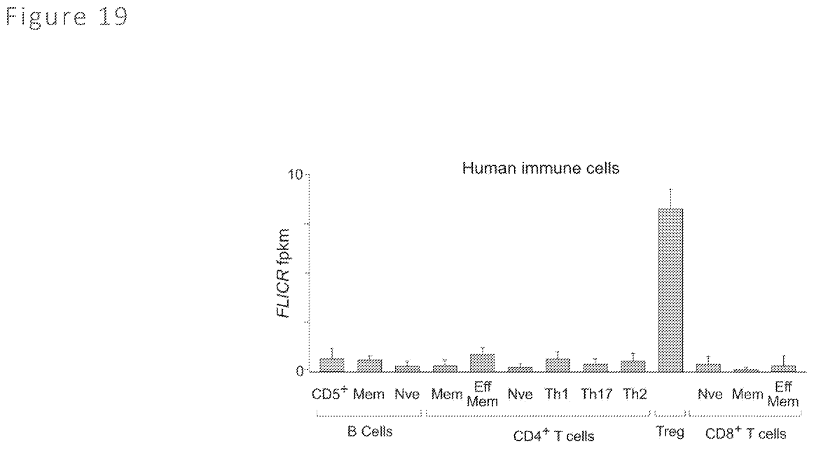

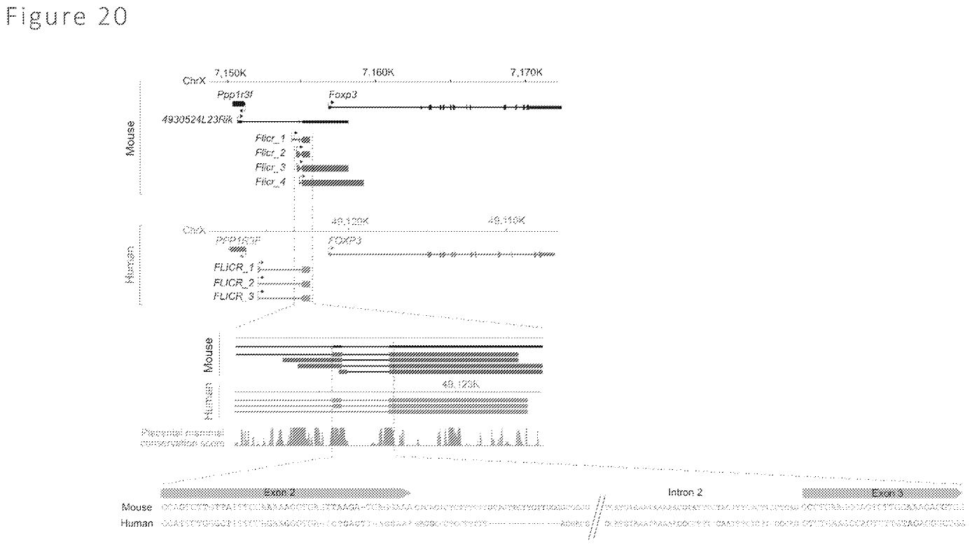

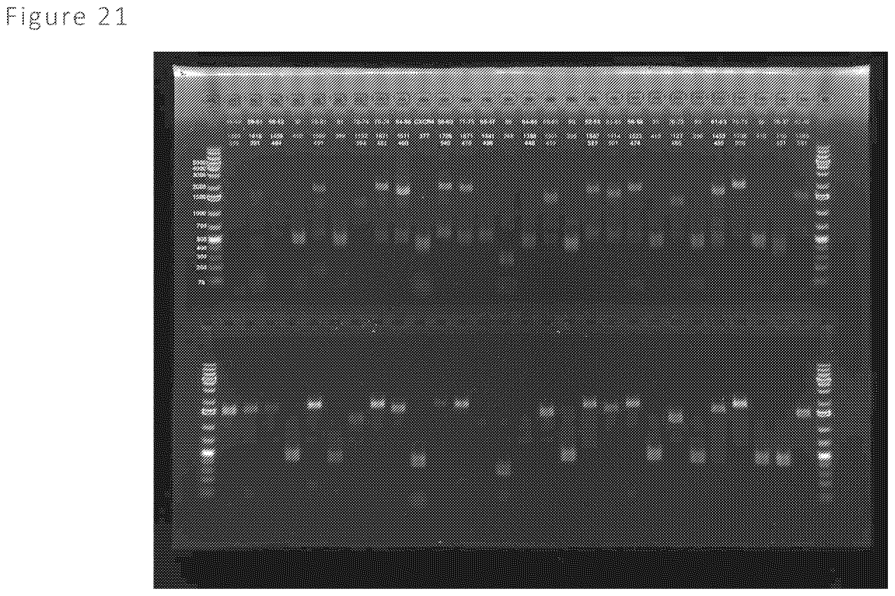

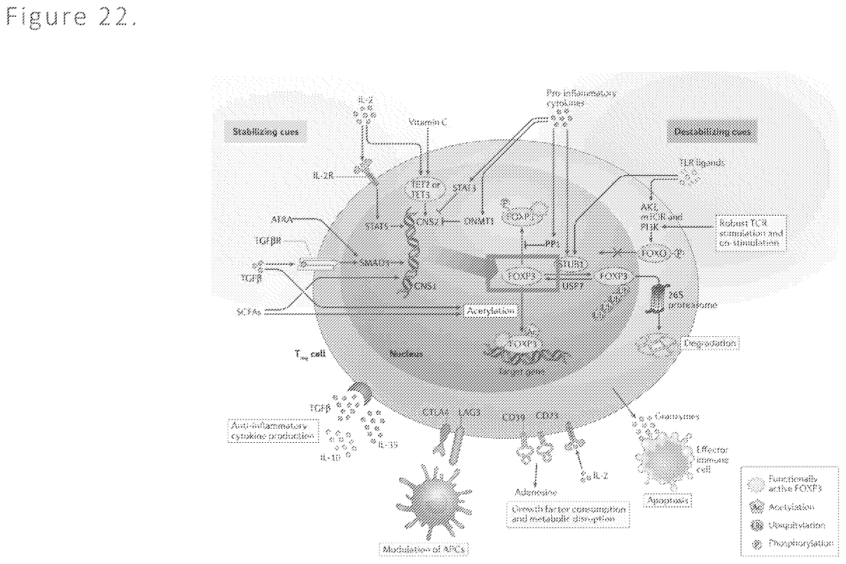

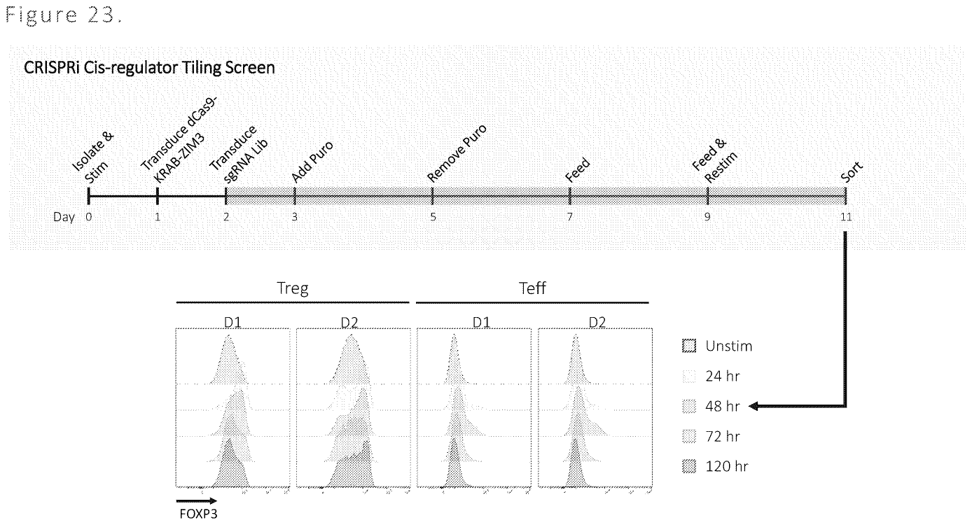

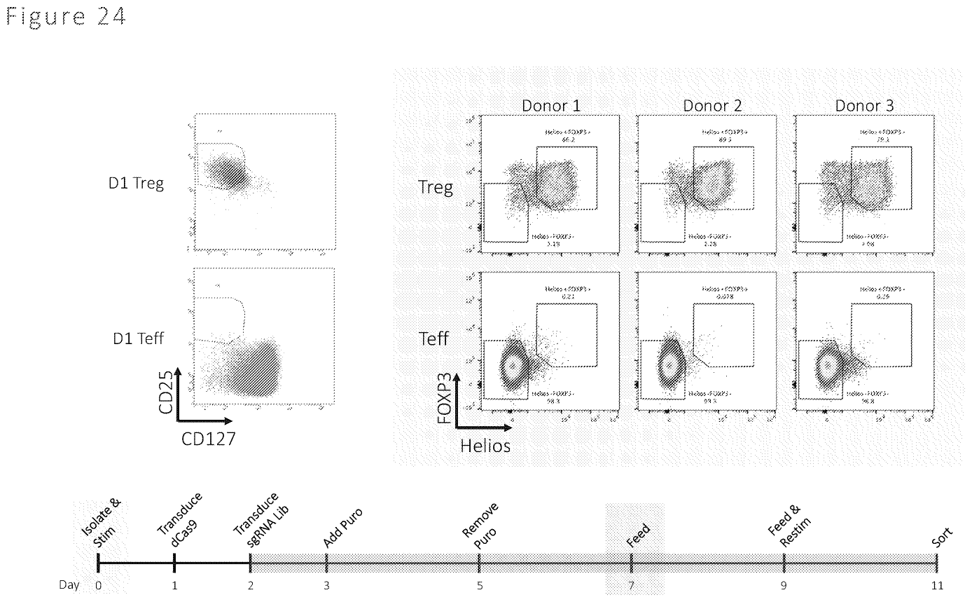

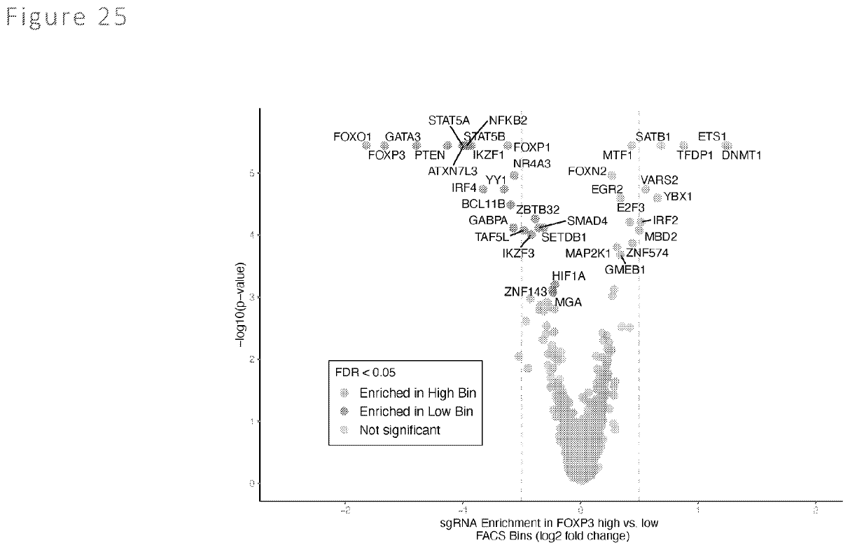

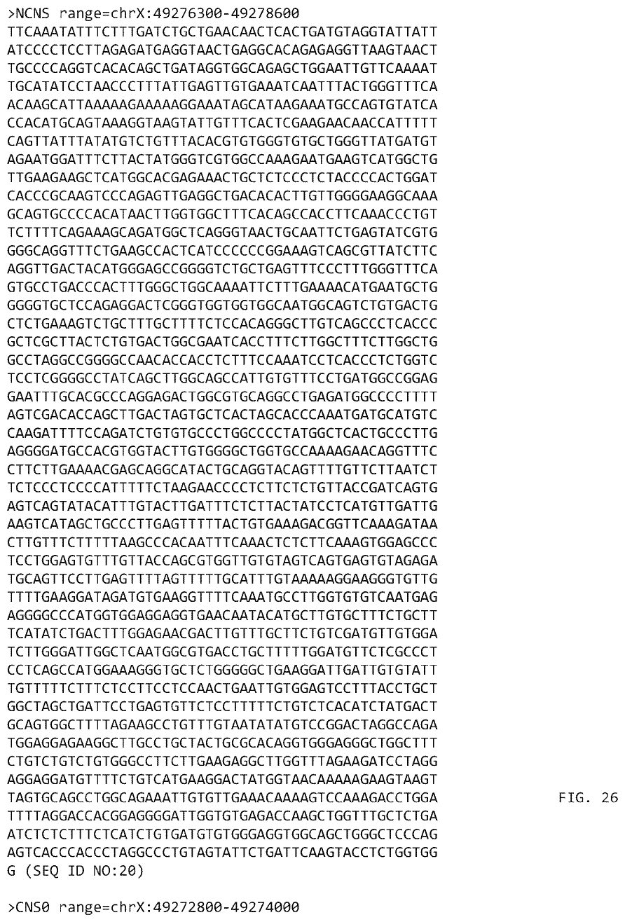

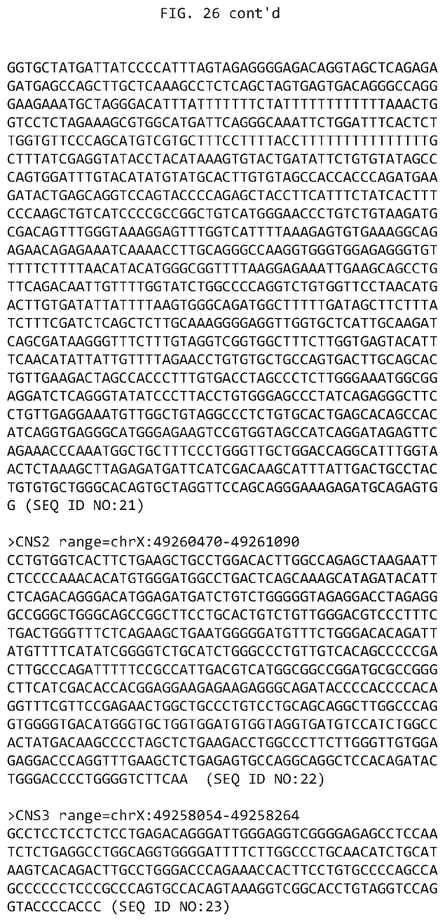

FIG. 17 shows trans-regulators of FOXP3 in Treg. The role of trans- and cis-regulators of FOXP3 under stability challenge is investigated using inflammatory cytokines, low IL-2 and/or antigenic ignorance

### Example 3

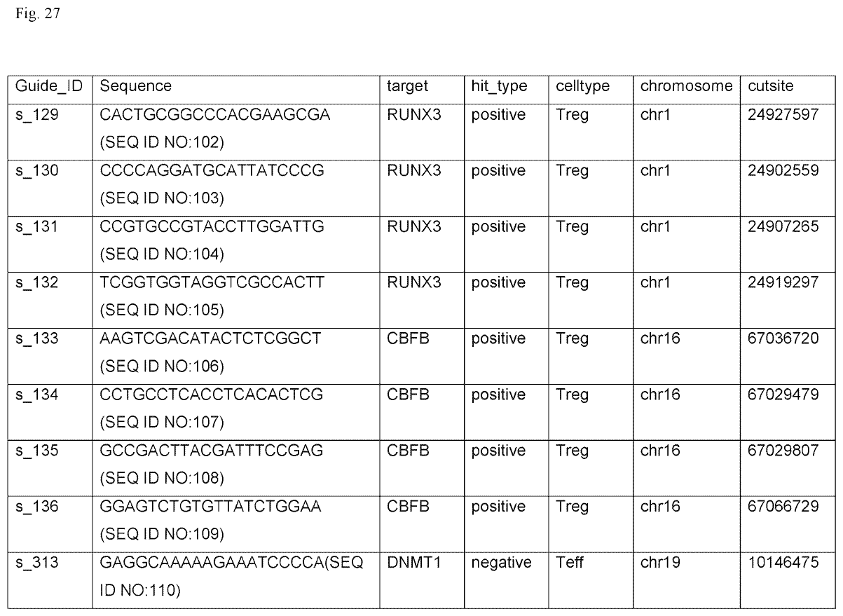

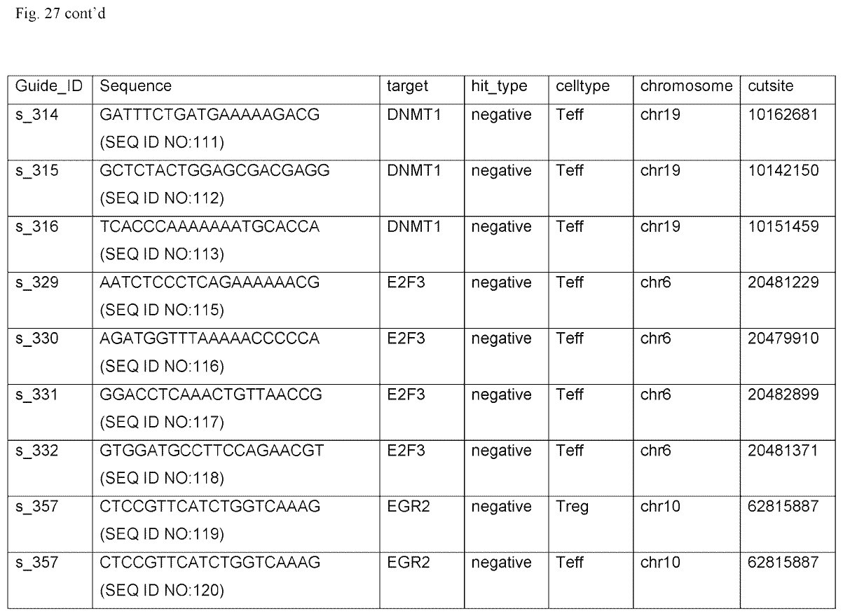

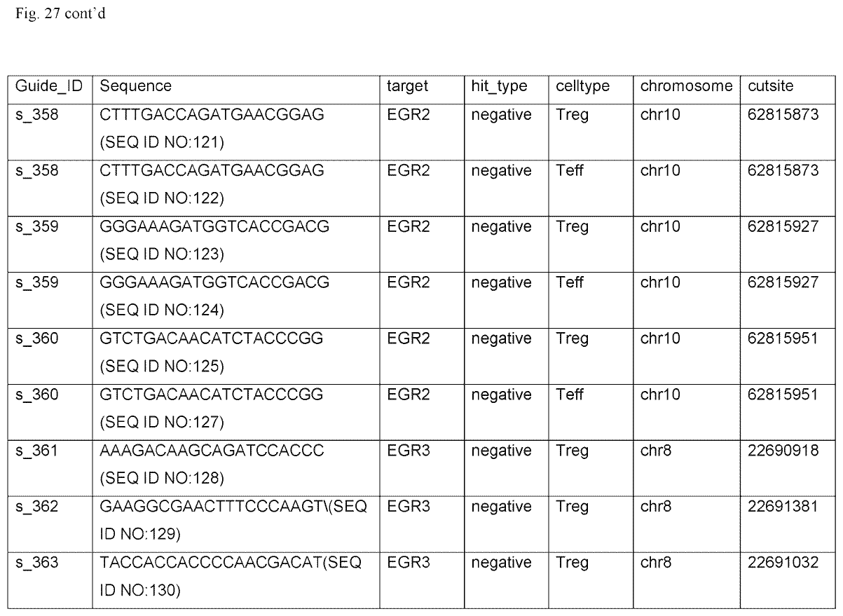

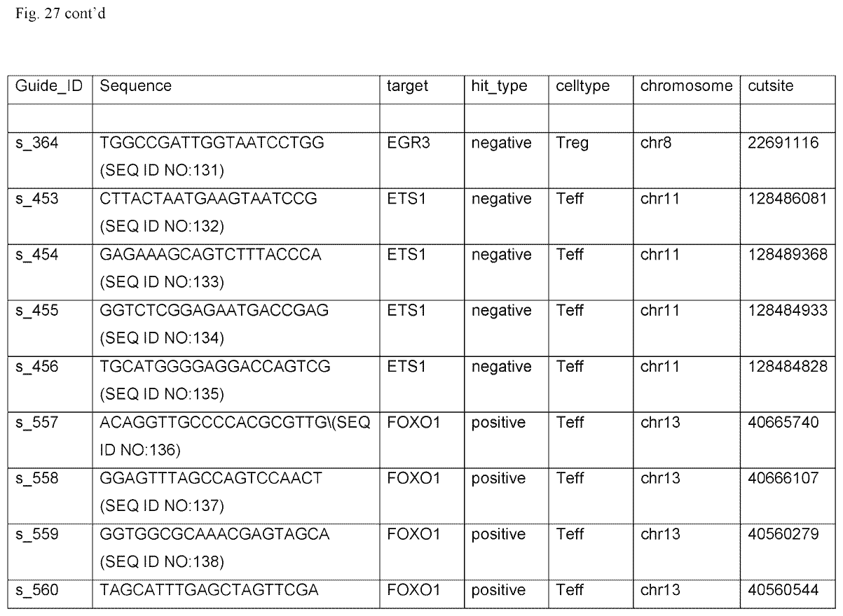

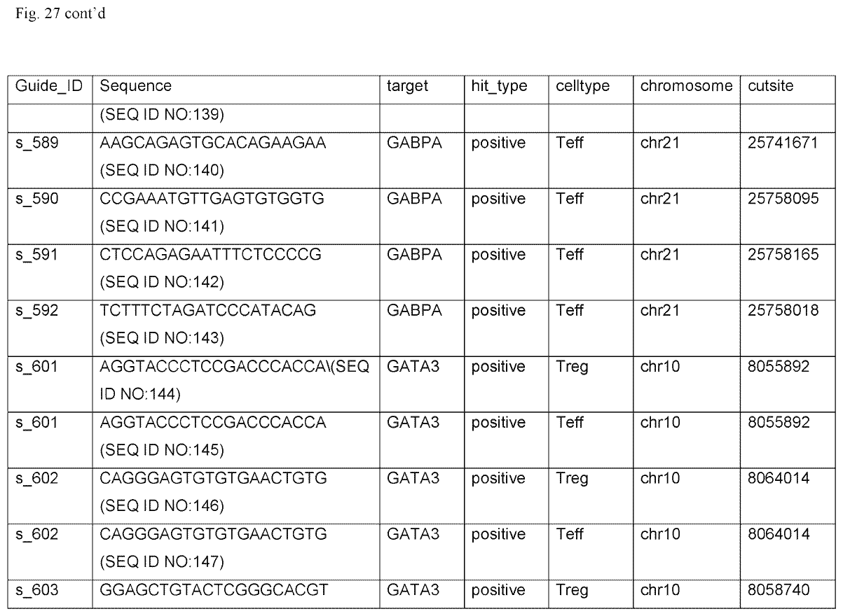

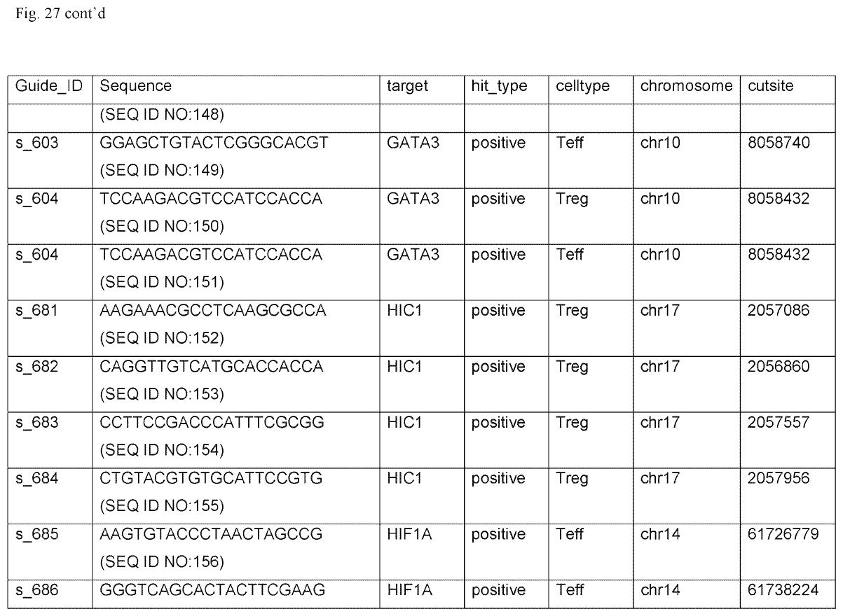

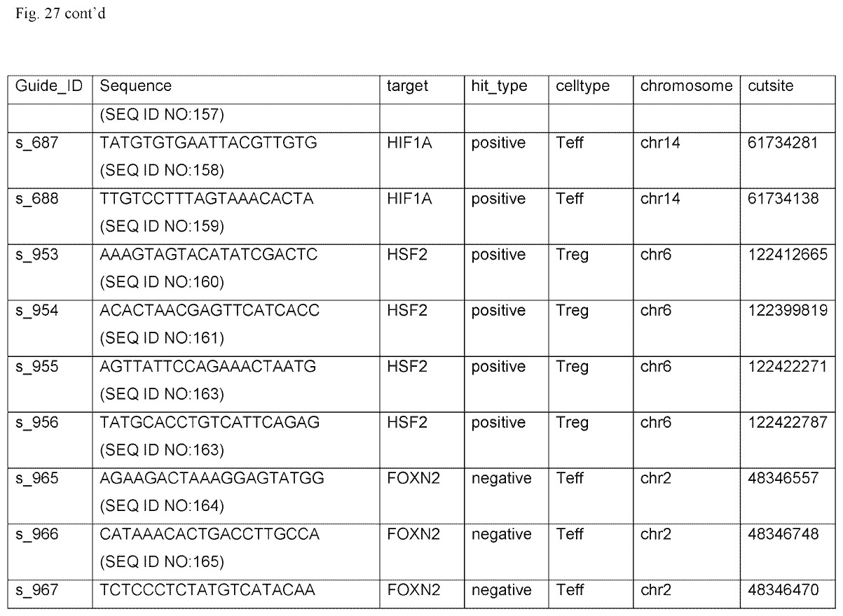

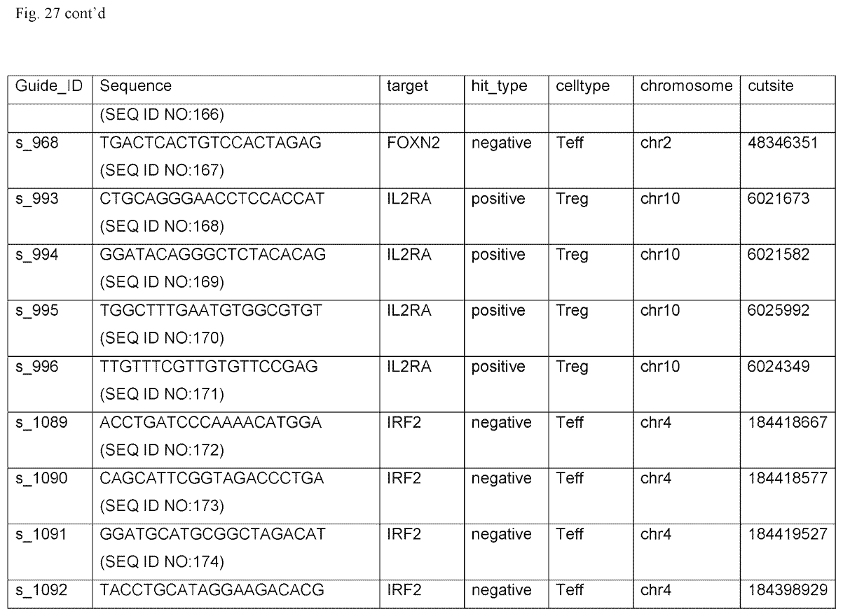

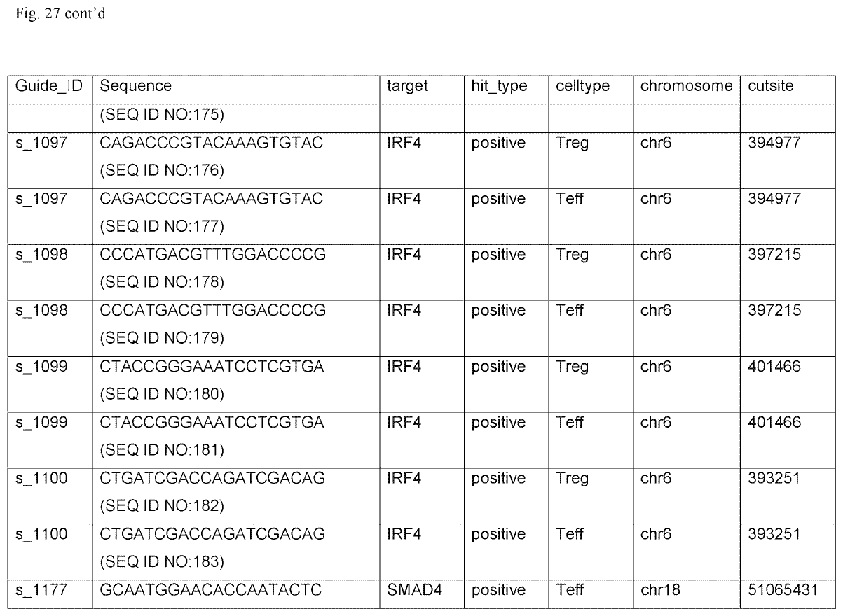

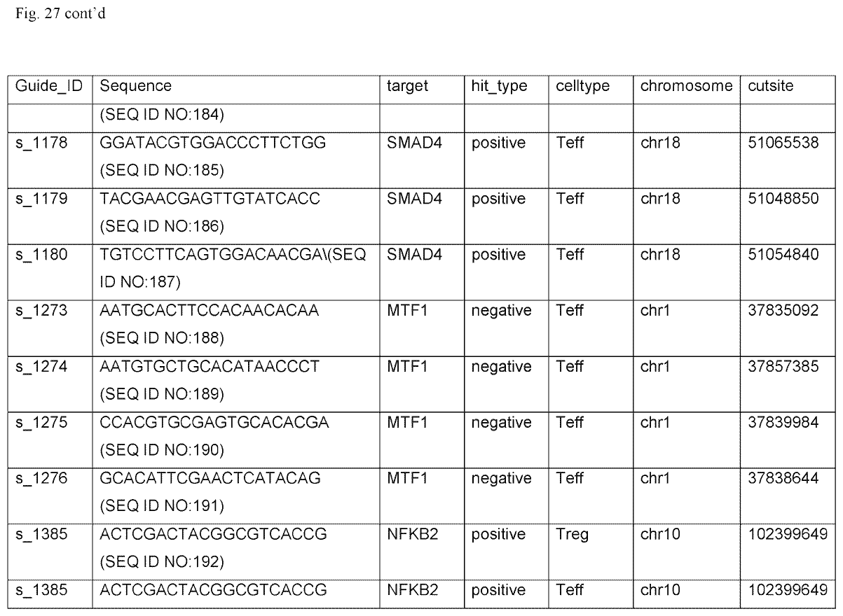

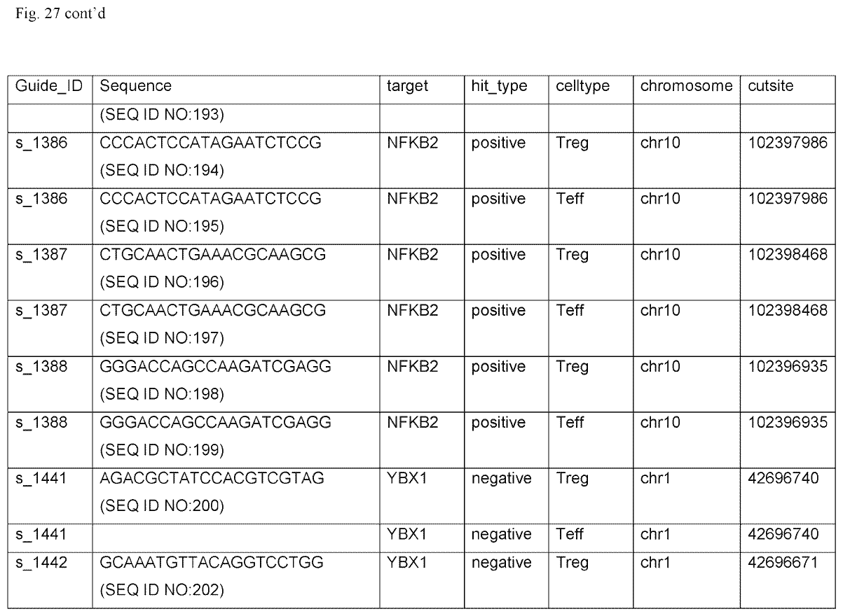

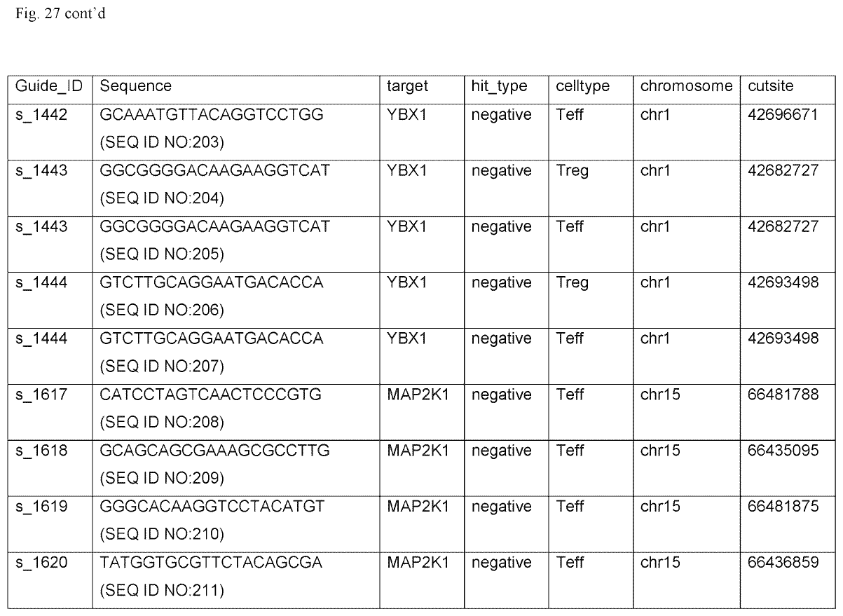

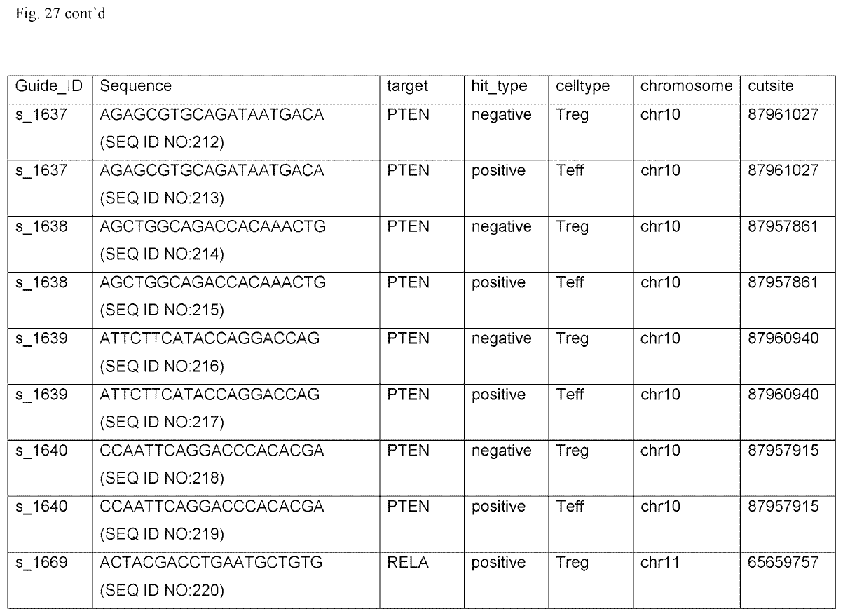

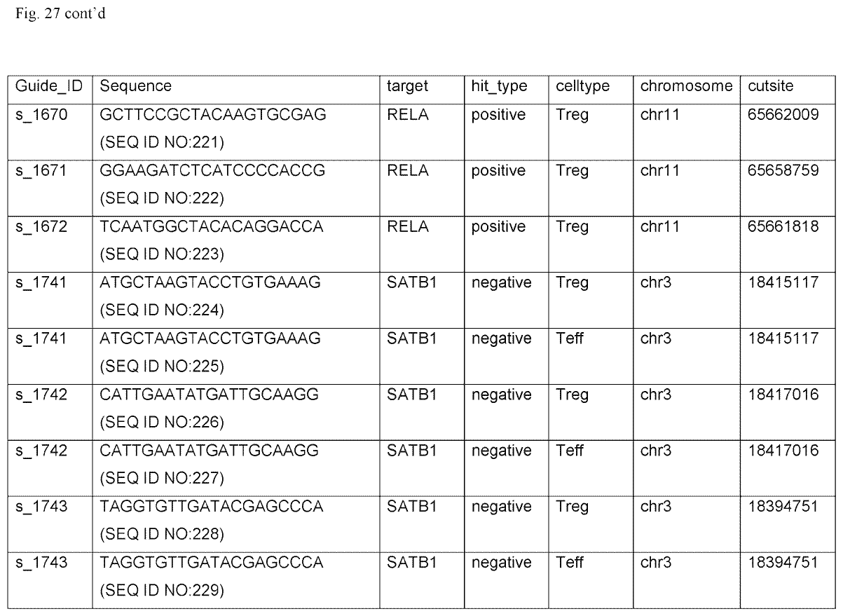

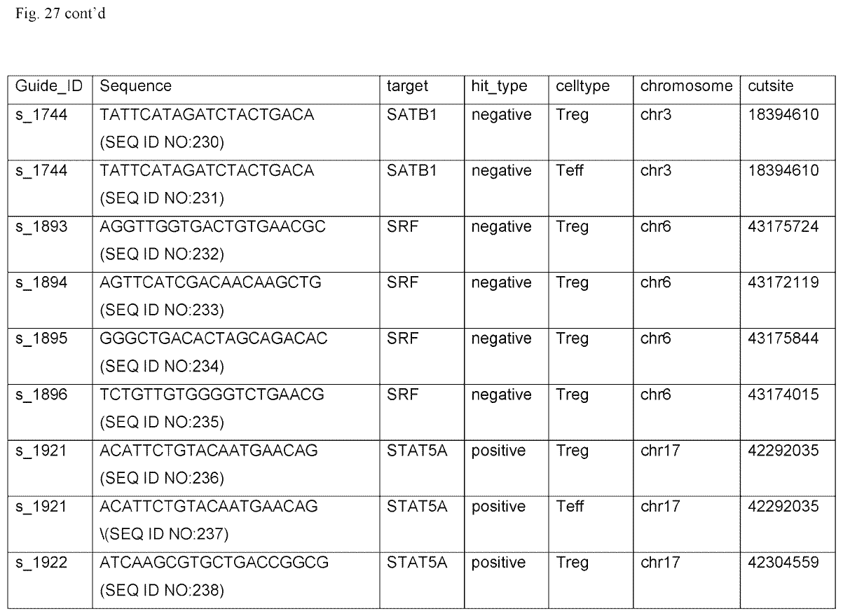

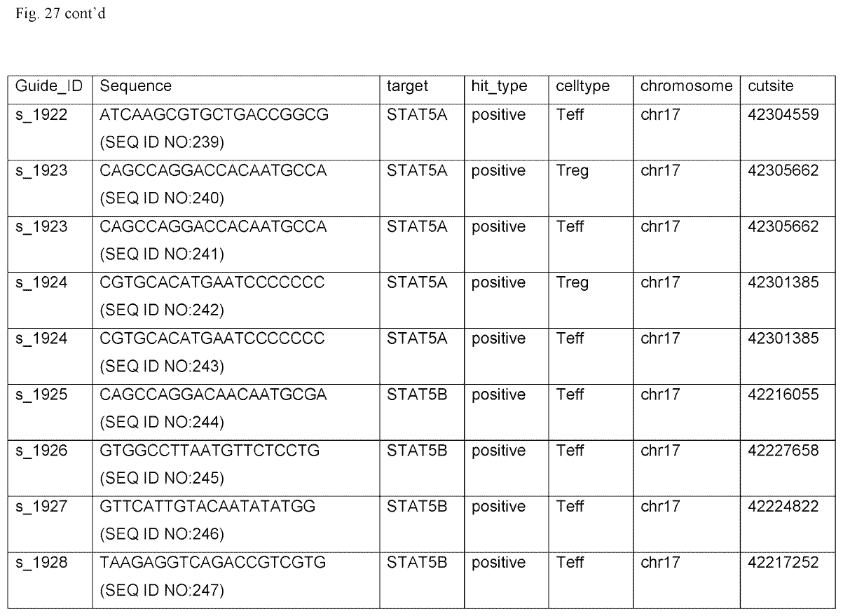

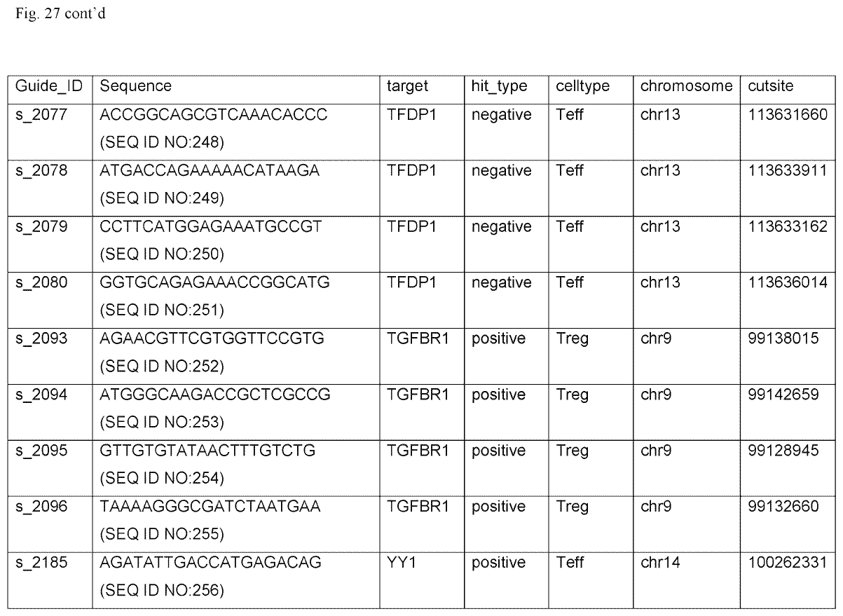

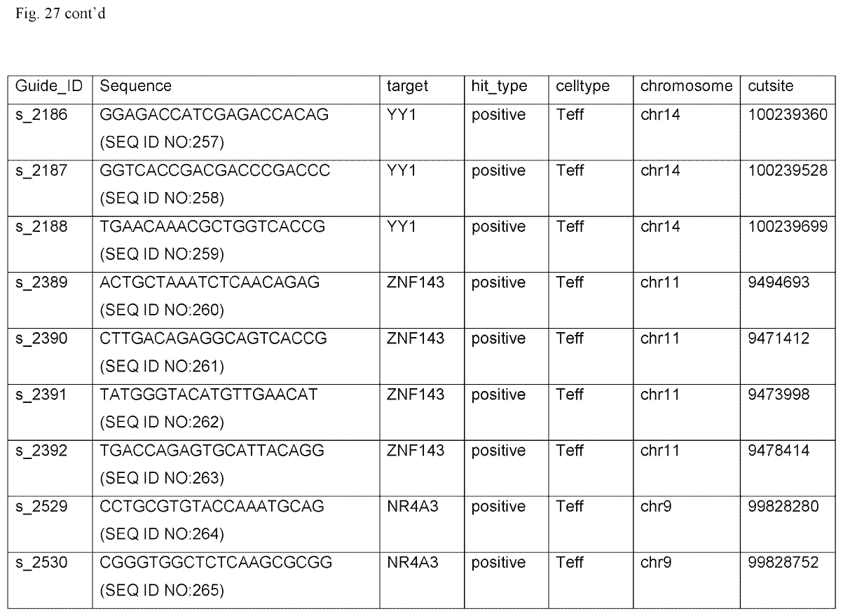

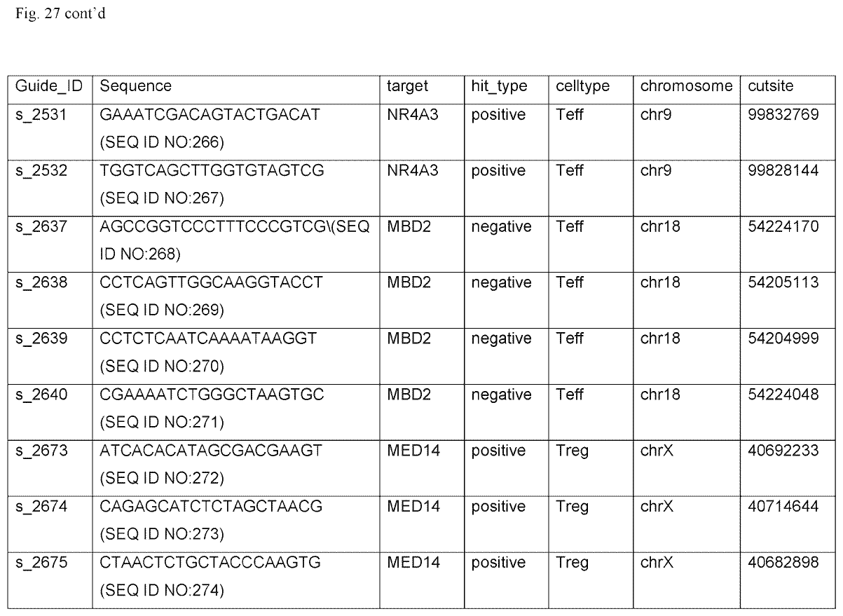

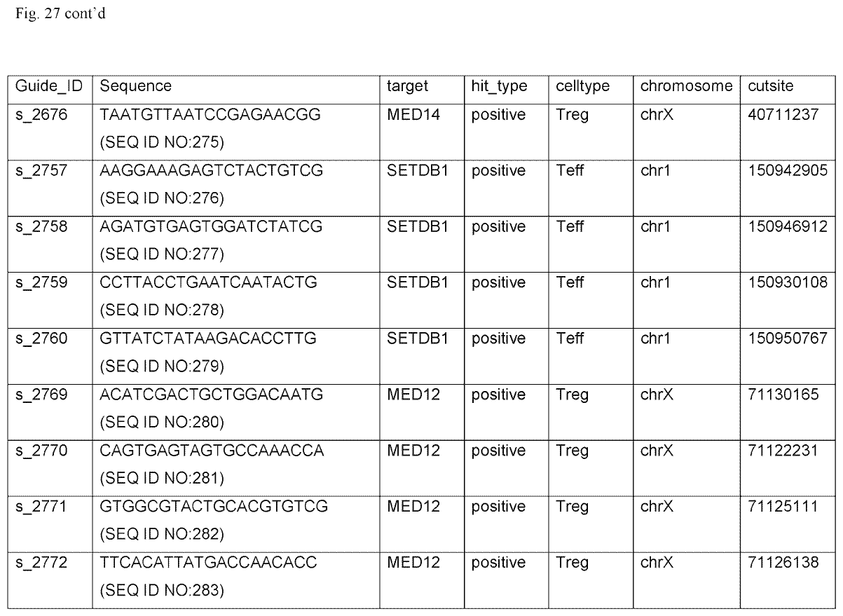

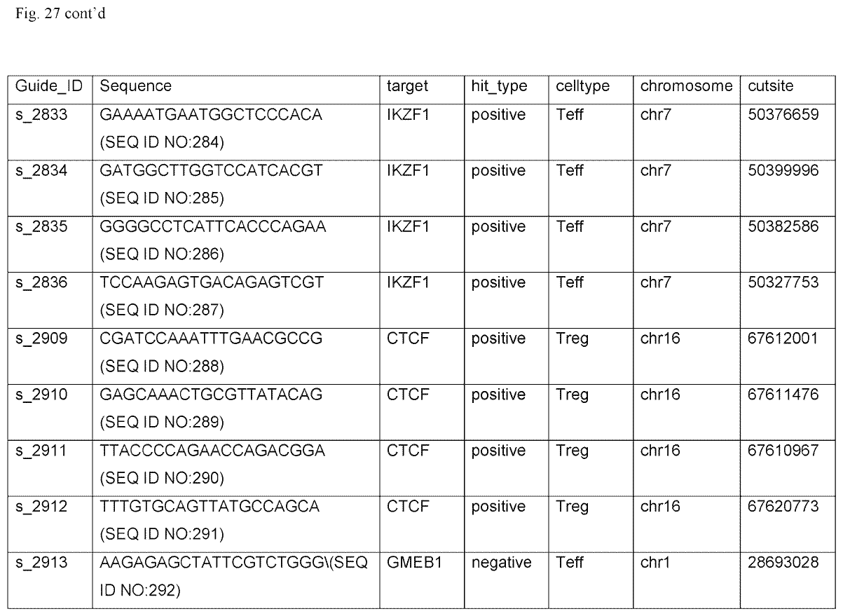

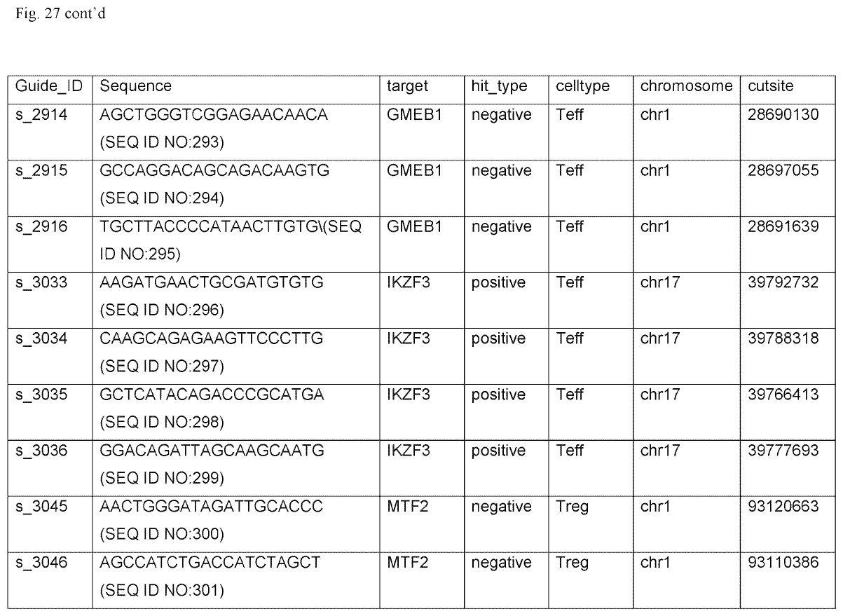

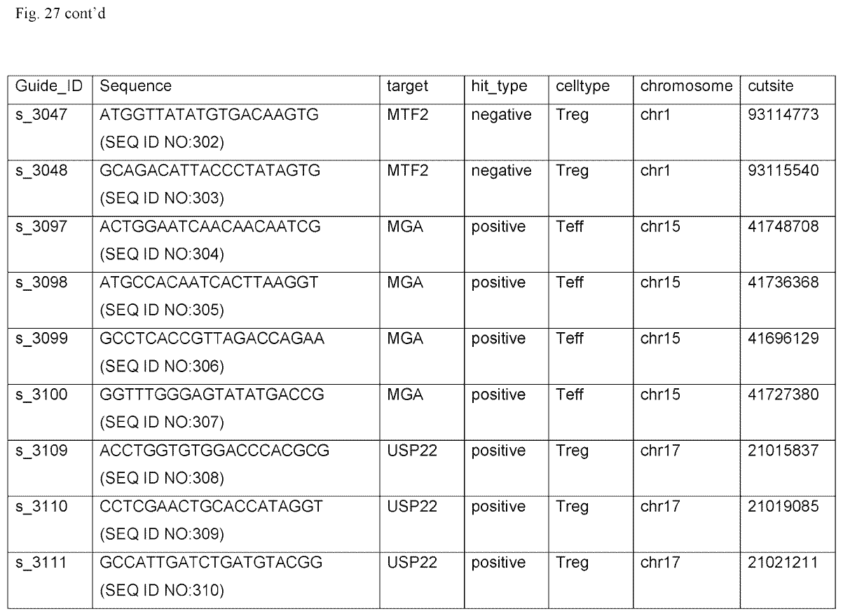

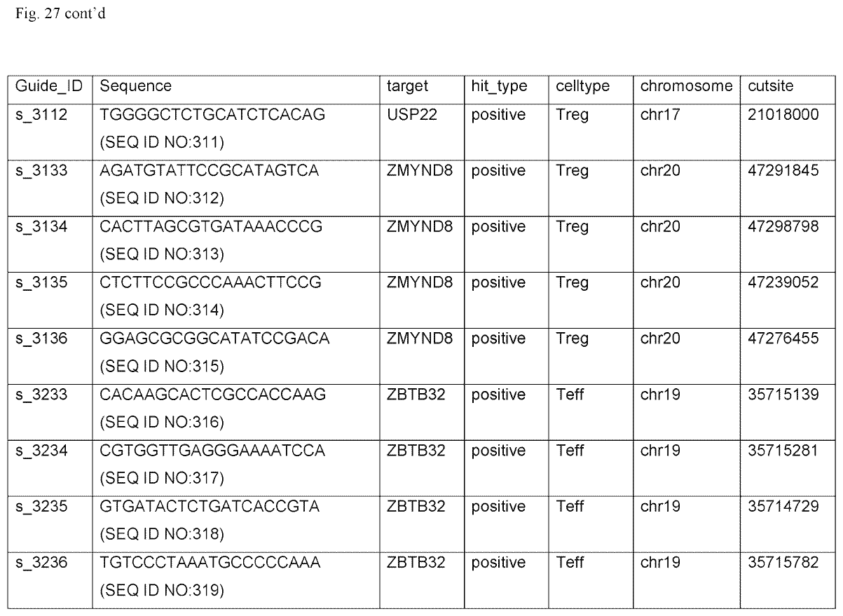

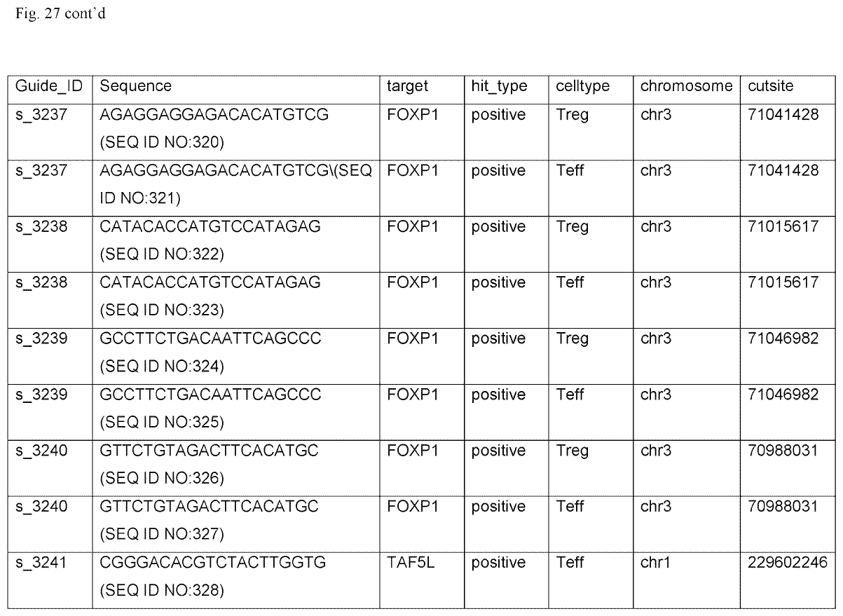

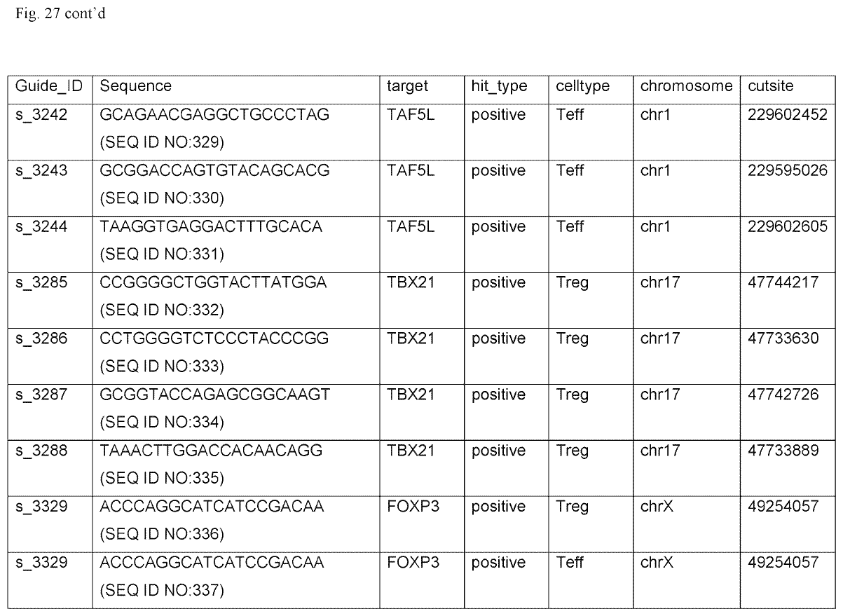

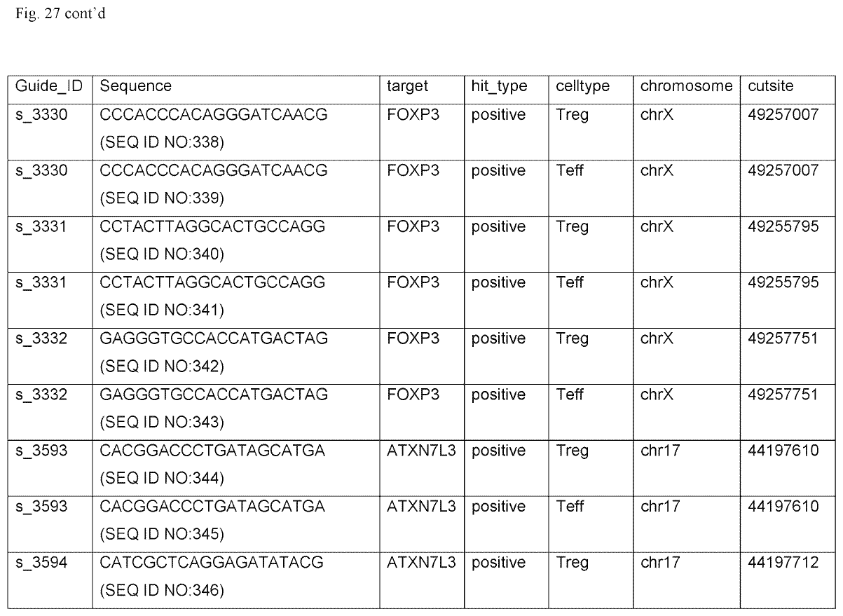

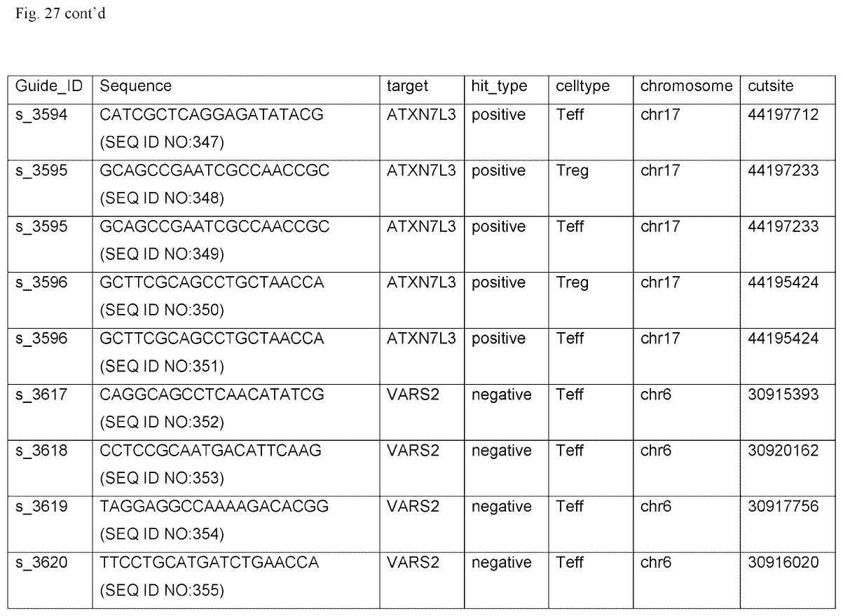

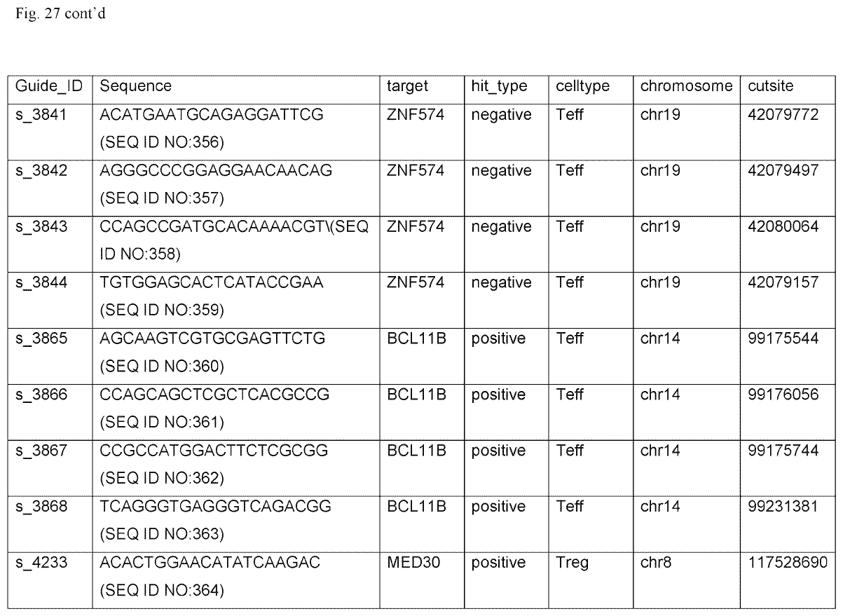

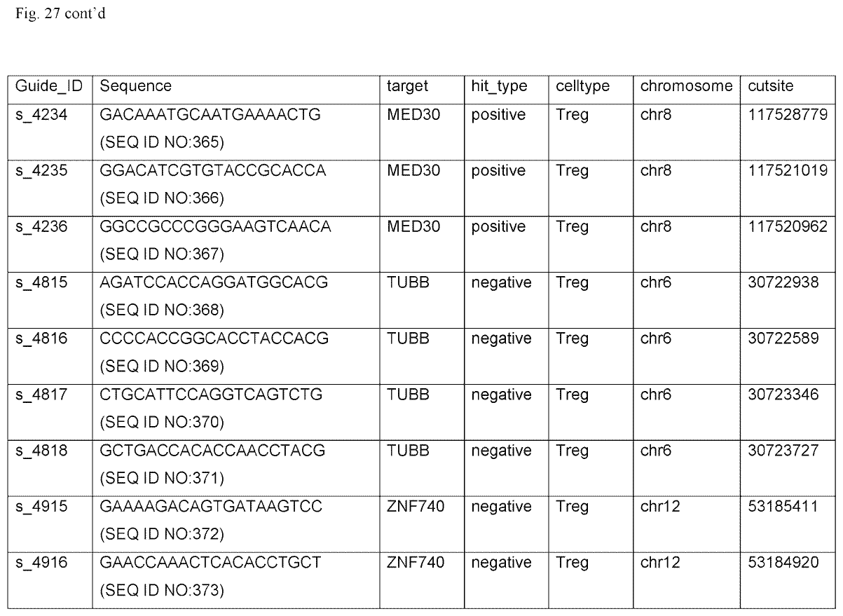

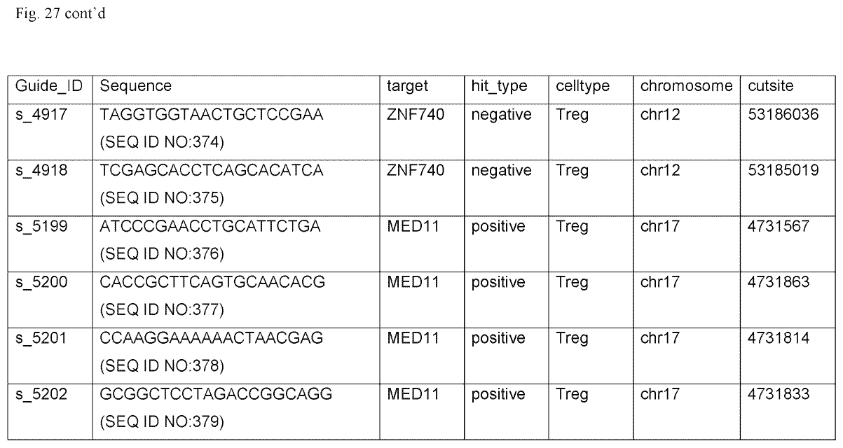

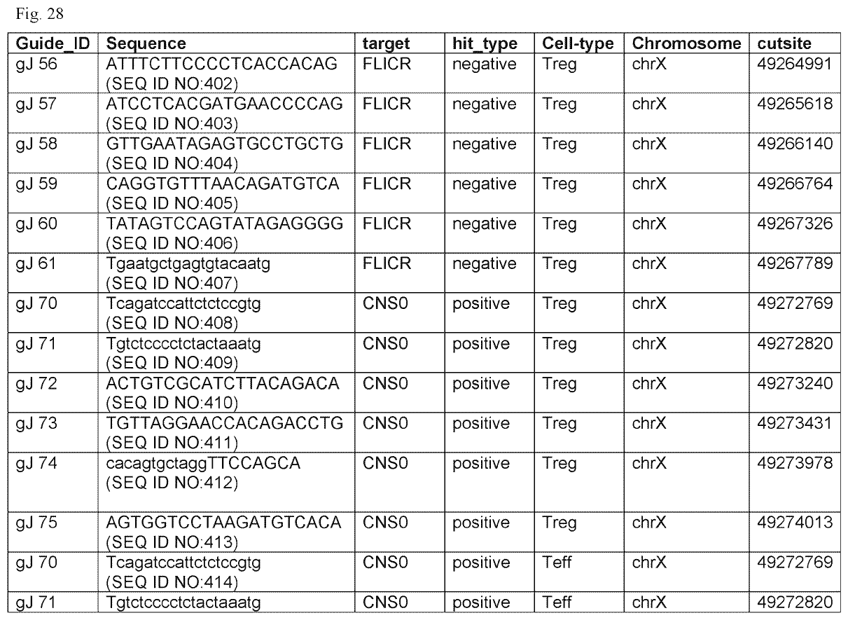

Altered expression of exemplary genes below was observed in Treg and/or Teff cells after gRNA exposure (also see FIG. 27)

| gene | hit_type | Cell type |
|---|---|---|
| FOXP3 | positive | Treg |
| MED12 | positive | Treg |
| FOXP1 | positive | Treg |
| ATXN7L3 | positive | Treg |
| GATA3 | positive | Treg |
| USP22 | positive | Treg |
| MED30 | positive | Treg |
| RUNX3 | positive | Treg |
| CBFB | positive | Treg |
| MED14 | positive | Treg |
| MED11 | positive | Treg |
| STAT5A | positive | Treg |
| NFKB2 | positive | Treg |
| IL2RA | positive | Treg |
| IRF4 | positive | Treg |
| HSF2 | positive | Treg |
| RELA | positive | Treg |
| ZMYND8 | positive | Treg |
| CTCF | positive | Treg |
| HIC1 | positive | Treg |
| TGFBR1 | positive | Treg |
| TBX21 | positive | Treg |
| MTF2 | negative | Treg |
| TUBB | negative | Treg |
| EGR2 | negative | Treg |
| SATB1 | negative | Treg |
| YBX1 | negative | Treg |
| ZNF740 | negative | Treg |
| EGR3 | negative | Treg |
| SRF | negative | Treg |
| PTEN | negative | Treg |
| FOXO1 | positive | Teff |
| PTEN | positive | Teff |
| GATA3 | positive | Teff |
| NFKB2 | positive | Teff |
| STAT5A | positive | Teff |
| ATXN7L3 | positive | Teff |
| FOXP3 | positive | Teff |
| STAT5B | positive | Teff |
| IKZF1 | positive | Teff |
| FOXP1 | positive | Teff |
| NR4A3 | positive | Teff |
| IRF4 | positive | Teff |
| YY1 | positive | Teff |
| BCL11B | positive | Teff |
| ZBTB32 | positive | Teff |
| GABPA | positive | Teff |
| SETDB1 | positive | Teff |
| SMAD4 | positive | Teff |
| TAF5L | positive | Teff |
| IKZF3 | positive | Teff |
| HIF1A | positive | Teff |
| ZNF143 | positive | Teff |
| MGA | positive | Teff |
| GMEB1 | negative | Teff |
| MTF1 | negative | Teff |
| IRF2 | negative | Teff |
| E2F3 | negative | Teff |
| MAP2K1 | negative | Teff |
| MBD2 | negative | Teff |
| ZNF574 | negative | Teff |
| VARS2 | negative | Teff |
| DNMT1 | negative | Teff |
| YBX1 | negative | Teff |
| EGR2 | negative | Teff |
| FOXN2 | negative | Teff |
| ETS1 | negative | Teff |
| TFDP1 | negative | Teff |
| SATB1 | negative | Teff |

### Example 4

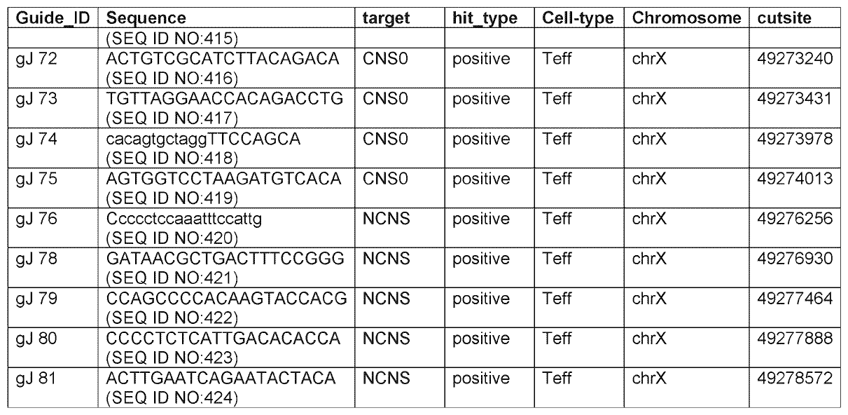

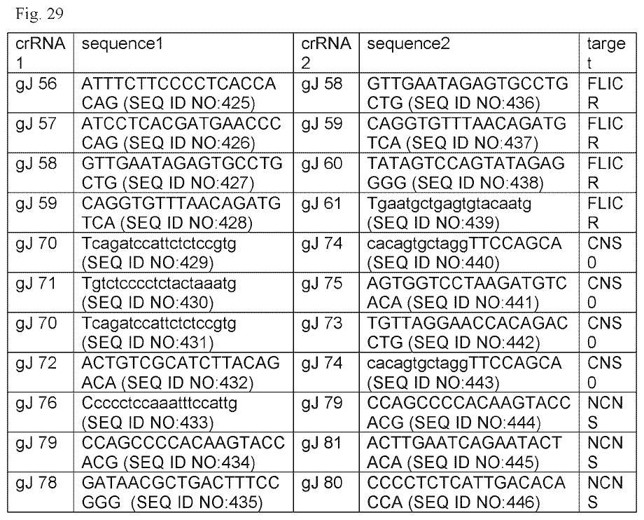

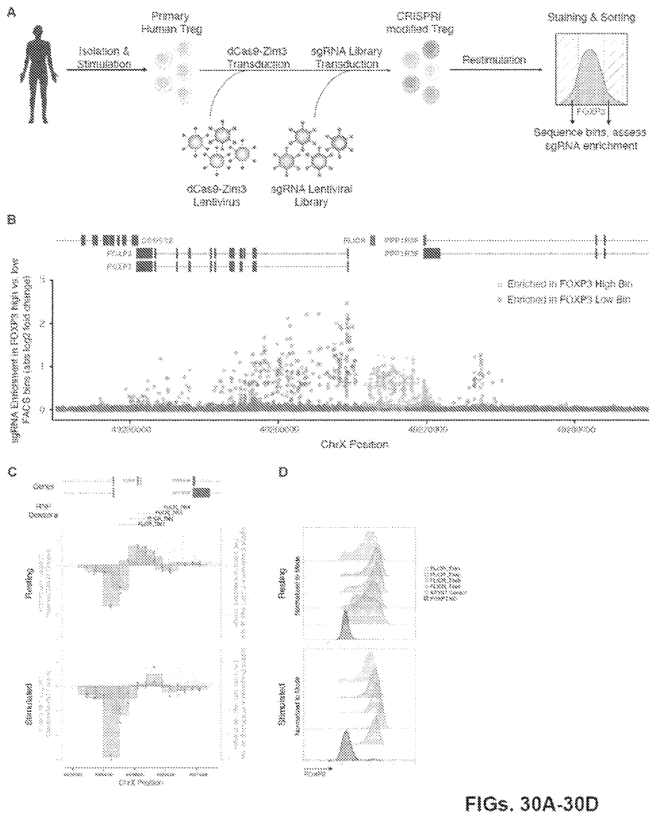

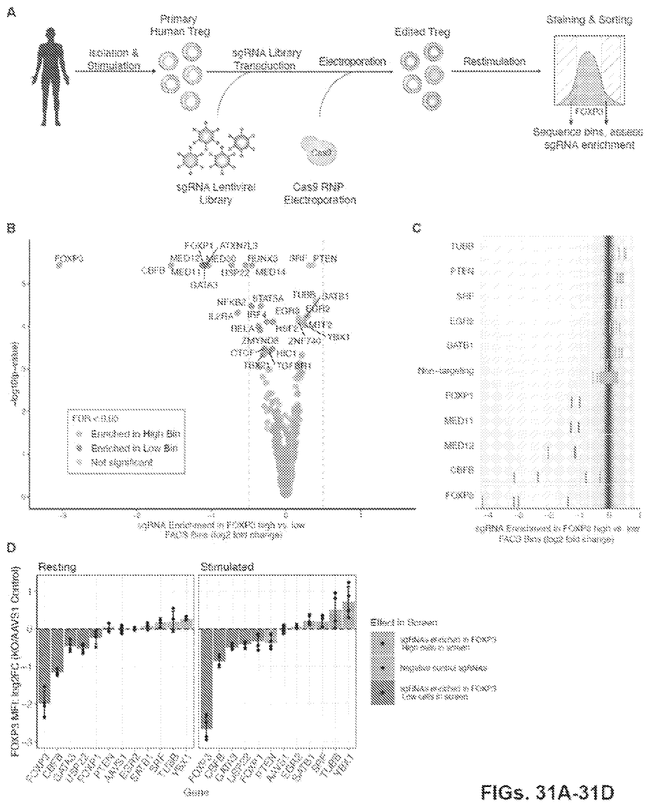

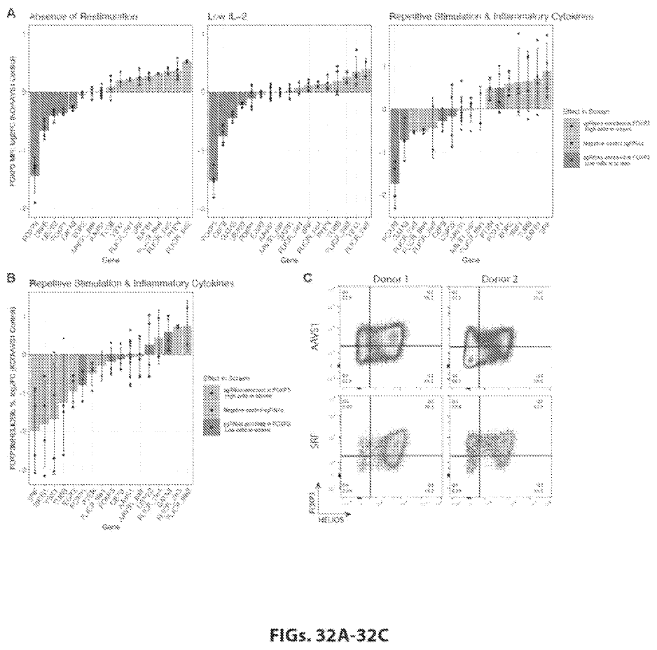

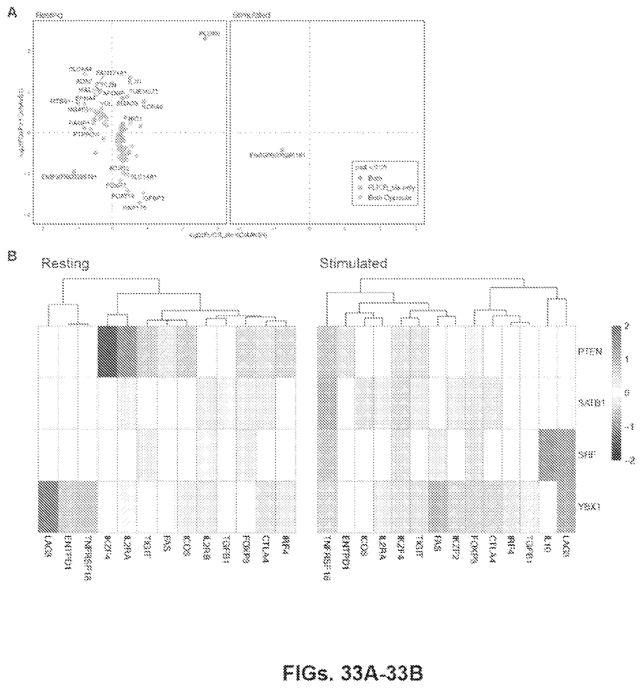

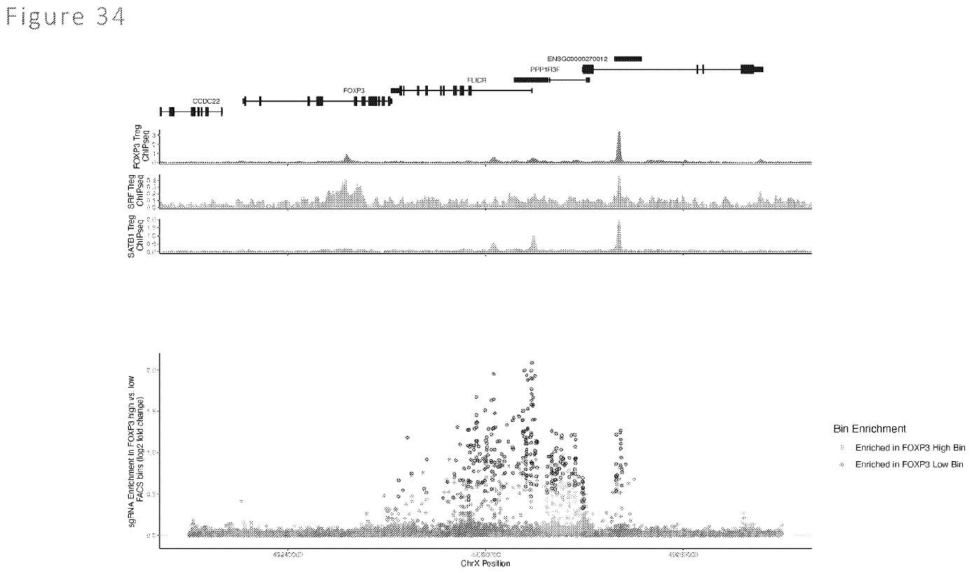

Exemplary paired crRNA sequences for deletions are shown below (see also FIG. 29; how paired when in use).

| name | sequence |
|---|---|
| gJ 56 | ATTTCTTCCCCTCACCACAG (SEQ ID NO: 1) |
| gJ 57 | ATCCTCACGATGAACCCCAG (SEQ ID NO: 2) |
| gJ 58 | GTTGAATAGAGTGCCTGCTG (SEQ ID NO: 3) |
| gJ 59 | CAGGTGTTTAACAGATGTCA (SEQ ID NO: 4) |
| gJ 60 | TATAGTCCAGTATAGAGGGG (SEQ ID NO: 5) |
| gJ 61 | Tgaatgctgagtgtacaatg (SEQ ID NO: 6) |

| crRNA1 | | crRNA2 | sequence2 |
|---|---|---|---|
| gJ 56 | ATTTCTTCCCCTCACCACA | gJ 58 | GTTGAATAGAGTGCCTGCTG |
| | G (SEQ ID NO: 1) | | (SEQ ID NO: 3) |
| gJ 57 | ATCCTCACGATGAACCCCA | gJ 59 | CAGGTGTTTAACAGATGTCA |
| | G (SEQ ID NO: 2) | | (SEQ ID NO: 4) |
| gJ 58 | GTTGAATAGAGTGCCTGCT | gJ 60 | TATAGTCCAGTATAGAGGGG |
| | G (SEQ ID NO: 3) | | (SEQ ID NO: 5) |
| gJ 59 | CAGGTGTTTAACAGATGTC | gJ 61 | Tgaatgctgagtgtacaatg |
| | A (SEQ ID NO: 4) | | (SEQ ID NO: 6) |

### Example 5

### FLICR Exons and Introns

>ENST00000651462.1 cdna:lncRNA

(SEQ ID NO: 7)
GGATGGGATCTCCTGCTCCTGTTCCTCCCGCTCCTGCCCACGGCGGGCTGCGCGGGACGT
AGCGCGCTGGGTGGTCGCAAAAGGAAGCCCAGCCGTCGTGTGAGGCCCGCACGTGCACCG
CCTTCTCGAAGGAGCGGTTCAGCACGCGTACCAACCCGCGCAGCACCGGCGGGCGGCCCC
CAGGCACCCACACCCCGGCACCCCCGGGGACCGCTCCGGGAGGCGGCAGCAGCGCCTCCA
GCTCCACCATGACGCGCCCCAAGCGCTCCAGACGGCCCGGCGCGGGCGGCAGCGAAAATG
TGGGGACCAGCCAGGCCATCAGGCCCATTCTGGGCTTTTCCAGAAGGGTCTGAAGCCAGT
CTTGTAGAGGGCTGGAGTGGTTGTTGGACGACTAGAACCCTGGGCTTTGCAGGGTGCTGG
GAGCTGGAAGTCTTCACCATGGCCTGTCAGGAGAAGCCCCTGGGGGCTGGTCTACCATGC
TGACCAGTGGTTGAATAGAGTGCCTGCTGAGGTCAAGAGATGCAGCCGCCAATTCCTGGA
CAACCTAAAAGCCTCACAAGCTTCAAGTCTCCAAGGACTCTCCAAATCCCAGGATCTCCC
TGCCATCACATCCTAGAATTTAAAACCTGAGAACATCAAGTTCAGGAAAGCTTGGGGATT
TCAAAGCCCAGCACCAAAGAAAGCGGGACCCACACTCGCCAGCTCTGTGATCTTGGGCAA
GTTGCTTGACTACTTATACCTCCGTTTCCCTCATCTGTATAACAGGAATACAATAATGAT
GAGATGGAGCTCAAGGGGCCATGGTGAGGATTGAACATGATCACTTGTGAGACATGTTCA
GCACTGTATCTGACCCATGGAAAATTCAAGATAAACATCAGCTACTGAGATGATGGCGGA
TATTTGGAATCCTAAATCCTTGGAAACTGGGGCTTTTTGAAGTAAAAGACCCCAAAGGCT
GAGGGCCTCAGAAGCATCAGGCCATGATGTTCCTGAAACAAGAGGGTCAGGGTCCCAATG
GGCCTCTGGGGTTCATCGTGAGGATGGATGCATTAATATTGGGGACCTGCTAGGGACCTT
CCCAGTGGGACAGTGGCTGGGTCAGGGCACTCAAGCCCTAAAACGTGATGAGGCGAGACT
TTTCTCTCTTTCCTCATTCAGTAACTGTCAGTAGATTCTGGGAGCCAGGGATTCTCCGAC
TCTTCAAGTCCATGAATTTTAGGGGATGACAGTGGGCTCTCCGCTTTCTCCTCCATGAAG
TAACTTACATGCCCCTCACCCTCTGTGGGAGGGGTGTTGCAGGGGGTGCAGAACTCCCCT
CGCCGGGTAGTTCAAGCAATGGGGACCATATCAATTCCATCTATAGGGAAACTGAGGCCT
GGAGTAGGGCGAGGCCTCTGGGAACCCAGCCCTATTCTGTCTCTTTCCCTGGCATTTCCC
ATCCACACATAGAGCTTCAGATTCTCTTTCTTTCCCCAGAGACCCTCAAATATCCTCTCA
CTCACAGAATGGTGTCTCTGCCTGCCTCGGGTTGGCCCTGTGATTTATTTTAGTTCTTTT
CCCTTGTTTTTTTTTTTTCAAACTCTATACACTTTTGTTTTAAAAACTGTGGTTTCTCAT
GAGCCCTATTATCTCATTGATACCTCTCACCTCTGTGGTGAGGGGAAGAAATCATATTTT
CAGATGACTCGTAAAGGGCAAAGAAAAAAACCCAAAATTTCAAAATTTCCGTTTAAGTCT
CATAATCAAGAAAAGGAGAAACACAGAGAGAGAGAAAAAAAAAACTATGAGAACCCCTCC
CCACCCCGTGATTATCAGCGCACACACTCATCGAAAAAAATTTGGATTATTAGAAGAGAG
AGGTCTGCGGCTTCCACACCGTACAGCGTGGTTTTTCTTCTCGGTATAAAAGCAAAGTTG
TTTTTGATACGTGACAGTTTCCCACAAGCCAGGCTGATCCTTTTCTGTCAGTCCACTTCA
CCAAGGTGAGTGTCCCTGCTCTCCCCTACCAGATGTGGGCCCCATTGGAGGAGATGGCAG
GGAGGTAGGCACGGCGGGGGGGTCAGGGGCCCTCTGGTACAGTGGGATGTACCCAGCTAC
CGTGATTCCAGCCAGGTAAGGTCTTTAAAAAAATAATAGAATAAATGGCAGAAGACTTAA
AGGTGAAGTACTTAATGCATGGGCAGGGGCAAAAATAAATGAATTCATCAATTGATACGT
AGATGGATAGGATTTTTAAAATATGTCAGCTGCCAGTCTTTATGGATTTCAGAGCTTTGA
ACAACTTGAAATGCTGTCCAGGTGCAACTGAAAATTTCAACTGCATCTCTCTCATCTCTT
TGTCTCTTAGCTCTTGATGCCTCTCATCCTTCTCTGTCTCTCTGCTCACCCTGTCTTGCT
CTTTTACATGTTTCTCTGCCTCTCTTTCTCCCGCCATCTGTCTGTCGCTACACTCAGACT
CTCTTTCCCCGCATCTCTCCTGGCCTCTGACCATCTCCCCTATCTCTGTCTCCTCCATCT
TGACCACACTCCATCGATCTCCTTTCCTCCATCTCTCCCGCTCTCTTTTCTTGCCTTCCC
CCACCTGCCTTCCCAGCCAGCATCCAGCCTAGAGCTTTGTACACAGCTGGCGTTTAATAA
TAGTAATGTCGCCGAGGACCTTTACATTTTATGAGAGTGAGCATCAGTTCTGTTCACCCT
AGTAGCAGGAGGATTGCAAGGTGCCGGGGCAAGCTGAGCACTTAACTGAACTTTGCTTGT
ATCGCTTTAGAGGAGACCCTTGGGGCGGGGGGGCAGGGAGCACTTCACACCATTGTCAAC
CGTAGTTTAGCTGTGTGGACCTACCCCAGGACTGCATGTGTGTGACCAGAGTCCATATTC
AGGAAGCAAACAGGTTGTTCAGGGCTGGGGTGAGCTCGATTCTGCAGGCTTAGCACAGCG
TTTGGCACAAAGTAAGTGCTCACGAAGTGTTAGCTATTAGTATTTCTGTAACCAAATTAG
AATCATGCTATATATTGGTTTTGTTCCTGCTTTATTCAATTAACAGTAGTGTTTTTGAAG
CTTACTAAAAATTGTTTGAACACATGATCTTTCATGATGGACAAGACTGTCCTCACATCA
GCATGAACTTTGATTGATTTGACCAAGTGCCGATTGTTGGACATTCCGGTTGTTTCTCGT
TTTTTTCTATAAACATCCCTGACTGCTTCCTTGGATTCCTGAAAAGGGAGTTGCTGGGGC
AAAGGGGGCGTGCATTTTTAAAGTTTATGACCCTCTCTGCTAATTCTCCTTCCAAAAGGT
TGTCCTATTTTATGCTCCCTCTACTTAAGCTAACCCTAGGTGAGTATTATTATGTTTTAA
TTATTTCCCAACATGACAGGTAAGAAAAACACATTATCACATTGTTGAAATTTGCATCTT
GTGATTACTTTTACTATTCATTGGCCATTCATGCTTTTTTTTTTTTTTTTTTGGTTGTCT
GGTCATGTCCTTACTCCTAATAGGGTGTTCATCTTATTTTTCCCTTATTGATTTGCCACA
GCTCTTTATTTGGAATGGTTATTAACCTTTCATCAGCCATTACATATATAGCAAATATTT
TCCCCAATTCATCATTTGCTTTTTTTCTATTTGTTTATGGTGTTTTTTGTGATGCTTATG
ATGGTTTTATACAGTCAAATAGGTTAGTCTTTTTTTCTGTGGCTTCTGTCTCTGGTTTTG
TGCTTAGAAAGTCCTTTCCTACTTGAAAATGAGATAAATGTTCACCTATGTTGGCTTCTA
>ENST00000651462.1 cds: lncRNA
>ENST00000651462.1 ENSE00003843437 exon: lncRNA
(SEQ ID NO: 8)
GGATGGGATCTCCTGCTCCTGTTCCTCCCGCTCCTGCCCACGGCGGGCTGCGCGGGACGT
AGCGCGCTGGGTGGTCGCAAAAGGAAGCCCAGCCGTCGTGTGAGGCCCGCACGTGCACCG
CCTTCTCGAAGGAGCGGTTCAGCACGCGTACCAACCCGCGCAGCACCGGCGGGCGGCCCC
CAGGCACCCACACCCCGGCACCCCCGGGGACCGCTCCGGGAGGCGGCAGCAGCGCCTCCA
GCTCCACCATGACGCGCCCCAAGCGCTCCAGACGGCCCGGCGCGGGCGGCAGCGAAAATG
TGGGGACCAG
>ENST00000651462.1 ENSE00003849356 exon: lncRNA
(SEQ ID NO: 9)
CCAGGCCATCAGGCCCATTCTGGGCTTTTCCAGAAGG
>ENST00000651462.1 ENSE00003849167 exon: lncRNA
(SEQ ID NO: 10)
GTCTGAAGCCAGTCTTGTAGAGGGCTGGAGTGGTTGTTGGACGACTAGAACCCTGGGCTT
TGCAGGGTGCTGGGAGCTGGAAGTCTTCACCATGGCCTGTCAGGAGAAGCCCCTGGGGGC
TGGTCTACCATGCTGACCAGTGGTTGAATAGAGTGCCTGCTGAGGTCAAGAGATGCAGCC
GCCAATTCCTGGACAACCTAAAAGCCTCACAAGCTTCAAGTCTCCAAGGACTCTCCAAAT
CCCAGGATCTCCCTGCCATCACATCCTAGAATTTAAAACCTGAGAACATCAAGTTCAGGA
AAGCTTGGGGATTTCAAAGCCCAGCACCAAAGAAAGCGGGACCCACACTCGCCAGCTCTG
TGATCTTGGGCAAGTTGCTTGACTACTTATACCTCCGTTTCCCTCATCTGTATAACAGGA
ATACAATAATGATGAGATGGAGCTCAAGGGGCCATGGTGAGGATTGAACATGATCACTTG
TGAGACATGTTCAGCACTGTATCTGACCCATGGAAAATTCAAGATAAACATCAGCTACTG
AGATGATGGCGGATATTTGGAATCCTAAATCCTTGGAAACTGGGGCTTTTTGAAGTAAAA
GACCCCAAAGGCTGAGGGCCTCAGAAGCATCAGGCCATGATGTTCCTGAAACAAGAGGGT
CAGGGTCCCAATGGGCCTCTGGGGTTCATCGTGAGGATGGATGCATTAATATTGGGGACC
TGCTAGGGACCTTCCCAGTGGGACAGTGGCTGGGTCAGGGCACTCAAGCCCTAAAACGTG
ATGAGGCGAGACTTTTCTCTCTTTCCTCATTCAGTAACTGTCAGTAGATTCTGGGAGCCA
GGGATTCTCCGACTCTTCAAGTCCATGAATTTTAGGGGATGACAGTGGGCTCTCCGCTTT
CTCCTCCATGAAGTAACTTACATGCCCCTCACCCTCTGTGGGAGGGGTGTTGCAGGGGGT
GCAGAACTCCCCTCGCCGGGTAGTTCAAGCAATGGGGACCATATCAATTCCATCTATAGG
GAAACTGAGGCCTGGAGTAGGGCGAGGCCTCTGGGAACCCAGCCCTATTCTGTCTCTTTC
CCTGGCATTTCCCATCCACACATAGAGCTTCAGATTCTCTTTCTTTCCCCAGAGACCCTC
AAATATCCTCTCACTCACAGAATGGTGTCTCTGCCTGCCTCGGGTTGGCCCTGTGATTTA
TTTTAGTTCTTTTCCCTTGTTTTTTTTTTTTCAAACTCTATACACTTTTGTTTTAAAAAC
TGTGGTTTCTCATGAGCCCTATTATCTCATTGATACCTCTCACCTCTGTGGTGAGGGGAA
GAAATCATATTTTCAGATGACTCGTAAAGGGCAAAGAAAAAAACCCAAAATTTCAAAATT
TCCGTTTAAGTCTCATAATCAAGAAAAGGAGAAACACAGAGAGAGAGAAAAAAAAAACTA
TGAGAACCCCTCCCCACCCCGTGATTATCAGCGCACACACTCATCGAAAAAAATTTGGAT
TATTAGAAGAGAGAGGTCTGCGGCTTCCACACCGTACAGCGTGGTTTTTCTTCTCGGTAT
AAAAGCAAAGTTGTTTTTGATACGTGACAGTTTCCCACAAGCCAGGCTGATCCTTTTCTG
TCAGTCCACTTCACCAAGGTGAGTGTCCCTGCTCTCCCCTACCAGATGTGGGCCCCATTG
GAGGAGATGGCAGGGAGGTAGGCACGGCGGGGGGGTCAGGGGCCCTCTGGTACAGTGGGA
TGTACCCAGCTACCGTGATTCCAGCCAGGTAAGGTCTTTAAAAAAATAATAGAATAAATG
GCAGAAGACTTAAAGGTGAAGTACTTAATGCATGGGCAGGGGCAAAAATAAATGAATTCA
TCAATTGATACGTAGATGGATAGGATTTTTAAAATATGTCAGCTGCCAGTCTTTATGGAT
TTCAGAGCTTTGAACAACTTGAAATGCTGTCCAGGTGCAACTGAAAATTTCAACTGCATC
TCTCTCATCTCTTTGTCTCTTAGCTCTTGATGCCTCTCATCCTTCTCTGTCTCTCTGCTC
ACCCTGTCTTGCTCTTTTACATGTTTCTCTGCCTCTCTTTCTCCCGCCATCTGTCTGTCG
CTACACTCAGACTCTCTTTCCCCGCATCTCTCCTGGCCTCTGACCATCTCCCCTATCTCT
GTCTCCTCCATCTTGACCACACTCCATCGATCTCCTTTCCTCCATCTCTCCCGCTCTCTT
TTCTTGCCTTCCCCCACCTGCCTTCCCAGCCAGCATCCAGCCTAGAGCTTTGTACACAGC
TGGCGTTTAATAATAGTAATGTCGCCGAGGACCTTTACATTTTATGAGAGTGAGCATCAG
TTCTGTTCACCCTAGTAGCAGGAGGATTGCAAGGTGCCGGGGCAAGCTGAGCACTTAACT
GAACTTTGCTTGTATCGCTTTAGAGGAGACCCTTGGGGGGGGGGGGCAGGGAGCACTTCA
CACCATTGTCAACCGTAGTTTAGCTGTGTGGACCTACCCCAGGACTGCATGTGTGTGACC
AGAGTCCATATTCAGGAAGCAAACAGGTTGTTCAGGGCTGGGGTGAGCTCGATTCTGCAG
GCTTAGCACAGCGTTTGGCACAAAGTAAGTGCTCACGAAGTGTTAGCTATTAGTATTTCT
GTAACCAAATTAGAATCATGCTATATATTGGTTTTGTTCCTGCTTTATTCAATTAACAGT
AGTGTTTTTGAAGCTTACTAAAAATTGTTTGAACACATGATCTTTCATGATGGACAAGAC
TGTCCTCACATCAGCATGAACTTTGATTGATTTGACCAAGTGCCGATTGTTGGACATTCC
GGTTGTTTCTCGTTTTTTTCTATAAACATCCCTGACTGCTTCCTTGGATTCCTGAAAAGG
GAGTTGCTGGGGCAAAGGGGGCGTGCATTTTTAAAGTTTATGACCCTCTCTGCTAATTCT
CCTTCCAAAAGGTTGTCCTATTTTATGCTCCCTCTACTTAAGCTAACCCTAGGTGAGTAT
TATTATGTTTTAATTATTTCCCAACATGACAGGTAAGAAAAACACATTATCACATTGTTG
AAATTTGCATCTTGTGATTACTTTTACTATTCATTGGCCATTCATGCTTTTTTTTTTTTT
TTTTTGGTTGTCTGGTCATGTCCTTACTCCTAATAGGGTGTTCATCTTATTTTTCCCTTA
TTGATTTGCCACAGCTCTTTATTTGGAATGGTTATTAACCTTTCATCAGCCATTACATAT
ATAGCAAATATTTTCCCCAATTCATCATTTGCTTTTTTTCTATTTGTTTATGGTGTTTTT
TGTGATGCTTATGATGGTTTTATACAGTCAAATAGGTTAGTCTTTTTTTCTGTGGCTTCT
GTCTCTGGTTTTGTGCTTAGAAAGTCCTTTCCTACTTGAAAATGAGATAAATGTTCACCT
ATGTTGGCTTCTA
>ENST00000651462.1 intron 1: lncRNA
(SEQ ID NO: 11)
GTAAAACCCCCCGCCAGCGGGGACGGGGCACAGCGGTGAGGGCTCGGGGCAAGCCTCCTC
TTCCTCCTCCCCTTCATCCCCATCCTCGCCATCGTCGTCCTCGTCGGCCCCGCCGCCGCC
GCCGCCATCTTGCCCGGCCGCCGCCGCCATCTTGCCCGCCCCGGGCCCGCCCCACGGCCG
GTAGCGGCGCAACTGCGCCAGCGGCAGCCCCAAGGCCTCGTCGGCGAACAGCACCCTCCG
CGGGGCCACCGCCGCCTCGACCGAGGTGCGGGGCTCACCCGCGGCCGGCGAGGGGGGCGC
GGAATGCCGCAGCGGGGGCTCCACAGGGGCCGTACGCGCCATATCGGCGGCGGCGGCGGC
ACCGACGGCACCGGCGGCGGGACCGGCGGGGGCAGGGCCTGAAGGGGCGGGCAGCCAATG
GGAAGGCCAGCGCGGAGGGGTACGGCTCACGGGGGCATAGGCGGTGAGATGAAGACAAGT
ATTGCAGAGGGCCAATGGGCAGCCATGCACGGATGACGTACAGCGAATTGAGAGCCGGAG
AAGCACGCAGCCAATGGGGATGCCCGGGAGTGGGCGGTGTAGGGGGCGGGATGATCGAGG
AATCAATGGAGAGGCCGGAATGGGGGTGGTGCGAAATCCATGATGAAGAAAACAGAAGCA
GACAGCTAATGCGGAAGTCTGAGCGAGAGAGGCATGTGGGCGGTGGGAAAAGTGGAATGG
CTACAGGCGATGAGGGCAGCCGGGAACGAACGCAGAGGCCCAAAAAGTGGGAGATCCCGC
GGGTTGCGTAGGCGGTGGAACGGTAGCTAGAGAGAGAGAACAGCCAATGGAGAGCCACGC
GTGGGATGACGACAATGGCTGCAGCGAGCCAATGGGGAGGCTTGTGAGCGTGCACTGCGG
CTTCGAAGGGGTGGTGCCAAGGTGGAGCTAGCCAATACGAAGGCTAAAAGGAGACCAAGA
GGCGGGTGTGTAGGGAGCCGAGCCAATGGAGGTGGAGGCTGAAGGCGGGAGCGAGGACGA
GAGCTGGCGGATAGCCAATGAGGGAGCCAGGGTGAAGGCGTAGAGACCTCAAAGGACAGT
ACTAGTATGGGATGAGCCAATGCCGAGGCTAGATAGTGCAGCGGGACGGAGGGAGGACTT
GGAGGCAAGATGGGGAAAAAAGCAGAGCCGACGGGGGCAGTGTAGGTGGTGTGAAGGGAG
GCATGAGTAGCCAATAAGGAGGCTGGAGAGGAGGACGCTGTGTAGACAGAGTCTGAGGTA
TTGGGTGGCCGGACAGCAGAACCATAGAACGCAGGCAGCCATTAGGAAAAATGTGCTTCT
CAGGGATGGGGTTACCCTCTCCCTAACCCTCCGTGACTCCCCATTGCCCCAGGAATACCC
AGAGCCCCTGAAGGCCTAGACCACACCTTGGTGTGTGAACTAGGAAAGTGAAAATGACTG
TCCTGTTGCAGTTAGTTACTTACTGGGTCCTCCCTGCCCAACTACAACCTTTTCTTGAGC
AGTGCCACAAGCCAAGAATGCCCCTGATTGTTAGCCGCAAGCACTCTCCAGGACTTGATA
CTATTGTTGAATGAATGGCCGAATAGGAAATTTAACTTTCTGAAGGATTCACTTTTTTAA
ATTTCAGCTCAGCAAACACTGACCTGACTGTGTGCTACCAGAAGTTGATTGGGCATATGG
GTAGCTTCTGACTAATGGAATCACAATCAAAGCCCTGTCCTTCTTGCCCAACCTGACACT
CCACACCCCACAGGGGCCTCACCTCCCTTCTTTCTGTCCCCCCATCTCTGCTTCTACTGT
TCCCTCTCCCAAGAGTGCTGTTCCTTCTGCTGCTTCCATCTAGTAAACTCCTTTCGCTCT
CCCTTTGGGGTCTCACCTTCTGTGGGAACTCAACTGCAGCTTTTGTCCGCTGAACACATC
CATTCATACATTCCTTCTCCATCCTGGGAAGATTTACTGAACACCTACTGAGTGCCAGGT
TCTGAGCATGCTGTTATCTCATTAGAACCTCACAGCATCCTAGGAGGGAGGTGATCCTTC
CAGCAGCCCCTCTCCCCAGGTCTCCATGTTCAGGATGTCCATGAGTTAGAAATGAATATG
TTCAGCTGCAAGTAACAGAAAAATCATAAAATAATGGGGGCTTAAGCAAGACAGAACTTT
ATTTTTTTCTCACCTAAAAGTTCAAGCTGGCAAATAGTCCAGGGACTCCTTCCTGCCCAC
CTGGTTTGGGCATTGTCTGTCCCGATCTGGCTCCAAGACTCTCAGTCTTCAACAAATCCT
TTTGGCTGTCTATTAAAAATATTTACCAAATCCATCCTCTTCTCACCACCTCCACTGCCA
GCACTATCACCAGTCACCTAGACTACTGCAGTAGCCAGCTCCTTGCTGGTCCCCTGCTTC
CACATTCAGGCCCTAGACCCCATTGTACACTCAGCATTCAGGGGCTTCCTGTTAAATCCT
AACACGGTTTGTGTTTCTCCTCTGCTTGCAACCCTGTCATGCCTTCCTCCTCACTCAGCG
TTGAAGCCAAAGTCCTTACCATGGCCTACAAGGTCCTGCGTGGTCTAACCTCCGAGTCCC
CTTTCTGACCTCACCTCCAGCTTCTCTTCTTGCTCTCTCCACACTAACCACACCAGCCCC
CTTGTAGACCTTGAACAAATCAGGTACATTCCCACCTCAGGACCTTTGCCCTGGCTGTTT
CCTTTGGAGTTCCTCAGATATCTAGCTGGCTCCCTCCCTCATCTCCTTCAAGTCTTTGTC
AGAGGCCCCTGTCTCTGGGGACTTTTCCTGACCCGTTTAATCTTGCCTACTCACCCCCAA
CTCCCAGCCCTCCCAGTCTCCTTCCCGAGAAGCCCTCCATGAGAGAGGATATAGTCCAGT
ATAGAGGGGAGGTAGCGACCTCTACCCCTCTCTGCCCTCCTGACCCCAGCCCCCATTCTG
CAAGGATCTTGGCTTTCAGCAGCCGTAGCATTTCCCTCTCACACCCTGGGCCTGCCCAAG
GGGCCAGCTCACATTCTCGGCCACCATCCTCACTTCGTTTGCCTCCTATATCTCTGGCCA
CTCTCTTTCATGGAGTGAGTGACAGTAAGGGACAGATGGTCTGGGGAAGGTATAAGGAGA
ATGCAGCCCACACCTCTGATCCTGAGAGAGAAAACTAGCCCCCTTTTCCAGGAGGGGGAC
AGGTGCTGGGGCAGGGAACAATGCCTCTGCTGATAGAACTGGCCTGGTTTATAATTACGC
AGGAGGTGTAGGGAACATTCCGACCCAAGTCTGCAGGGAGCAAGGGGTTATTAACTATGG
GAACATCAGTGCAAGGGATCATTAACCTTAGGACCATCACTGCAAGGGGTCATTAACCTT
GGGGCCATCACTAGAATCAGCTCCCCAGGACTCCTTCCTGACATGCATGCTCCTCACCCC
CTCCCTTGCTCCCTCCCACATGCCGTGACATCTGTTAAACACCTGCTGTTCACAGGGTCC
CTGCAGGCCTCCAAGCAGGGGACCTCATCCCACCTTGGTGCCTCCCCTGGCTGTCCAACA
CACCCAAGCCATTTTTGGGTCTCAGATGTGTCCCACCAACTCAGAAGCCTCAACCAGCGA
AAGTTTTGTTTTGAACTAATGGTGGGGAGGGTTGGGTGTTTTTTTTTTTTTTTAATTGTT
GTTGGCTGATTTTTTTAAGGTCTTTTTTTTTCTATTCTTTTTTTTTTTTTTTCTTGTCTT
CACAAAG
>ENST00000651462.1 intron 2: lncRNA
(SEQ ID NO: 12)
GTGAGTCTGAGTTGAGGAAAGAGGCCCTCCTTTTTACGAGGGCTTAGAAGGTTGGGGGAG
GAAAGGGGGCAAAGGGGTGAAGGGGTGGATCTTGGCCACCAGATTTGTACCATGTAAATA
AAAAGCCCTTTCCAATTTCTCTTCCCCAG
>X dna: chromosome chromosome: GRCh38: X: 49262866: 49270521:
−1
(SEQ ID NO: 13)
GGATGGGATCTCCTGCTCCTGTTCCTCCCGCTCCTGCCCACGGCGGGCTGCGCGGGACGT
AGCGCGCTGGGTGGTCGCAAAAGGAAGCCCAGCCGTCGTGTGAGGCCCGCACGTGCACCG
CCTTCTCGAAGGAGCGGTTCAGCACGCGTACCAACCCGCGCAGCACCGGCGGGCGGCCCC
CAGGCACCCACACCCCGGCACCCCCGGGGACCGCTCCGGGAGGCGGCAGCAGCGCCTCCA
GCTCCACCATGACGCGCCCCAAGCGCTCCAGACGGCCCGGCGCGGGCGGCAGCGAAAATG
TGGGGACCAGGTAAAACCCCCCGCCAGCGGGGACGGGGCACAGCGGTGAGGGCTCGGGGC
AAGCCTCCTCTTCCTCCTCCCCTTCATCCCCATCCTCGCCATCGTCGTCCTCGTCGGCCC
CGCCGCCGCCGCCGCCATCTTGCCCGGCCGCCGCCGCCATCTTGCCCGCCCCGGGCCCGC
CCCACGGCCGGTAGCGGCGCAACTGCGCCAGCGGCAGCCCCAAGGCCTCGTCGGCGAACA
GCACCCTCCGCGGGGCCACCGCCGCCTCGACCGAGGTGCGGGGCTCACCCGCGGCCGGCG
AGGGGGGCGCGGAATGCCGCAGCGGGGGCTCCACAGGGGCCGTACGCGCCATATCGGCGG
CGGCGGCGGCACCGACGGCACCGGCGGCGGGACCGGCGGGGGCAGGGCCTGAAGGGGCGG
GCAGCCAATGGGAAGGCCAGCGCGGAGGGGTACGGCTCACGGGGGCATAGGCGGTGAGAT
GAAGACAAGTATTGCAGAGGGCCAATGGGCAGCCATGCACGGATGACGTACAGCGAATTG
AGAGCCGGAGAAGCACGCAGCCAATGGGGATGCCCGGGAGTGGGCGGTGTAGGGGGCGGG
ATGATCGAGGAATCAATGGAGAGGCCGGAATGGGGGTGGTGCGAAATCCATGATGAAGAA
AACAGAAGCAGACAGCTAATGCGGAAGTCTGAGCGAGAGAGGCATGTGGGCGGTGGGAAA
AGTGGAATGGCTACAGGCGATGAGGGCAGCCGGGAACGAACGCAGAGGCCCAAAAAGTGG
GAGATCCCGCGGGTTGCGTAGGCGGTGGAACGGTAGCTAGAGAGAGAGAACAGCCAATGG
AGAGCCACGCGTGGGATGACGACAATGGCTGCAGCGAGCCAATGGGGAGGCTTGTGAGCG
TGCACTGCGGCTTCGAAGGGGTGGTGCCAAGGTGGAGCTAGCCAATACGAAGGCTAAAAG
GAGACCAAGAGGCGGGTGTGTAGGGAGCCGAGCCAATGGAGGTGGAGGCTGAAGGCGGGA
GCGAGGACGAGAGCTGGCGGATAGCCAATGAGGGAGCCAGGGTGAAGGCGTAGAGACCTC
AAAGGACAGTACTAGTATGGGATGAGCCAATGCCGAGGCTAGATAGTGCAGCGGGACGGA
GGGAGGACTTGGAGGCAAGATGGGGAAAAAAGCAGAGCCGACGGGGGCAGTGTAGGTGGT
GTGAAGGGAGGCATGAGTAGCCAATAAGGAGGCTGGAGAGGAGGACGCTGTGTAGACAGA
GTCTGAGGTATTGGGTGGCCGGACAGCAGAACCATAGAACGCAGGCAGCCATTAGGAAAA
ATGTGCTTCTCAGGGATGGGGTTACCCTCTCCCTAACCCTCCGTGACTCCCCATTGCCCC
AGGAATACCCAGAGCCCCTGAAGGCCTAGACCACACCTTGGTGTGTGAACTAGGAAAGTG
AAAATGACTGTCCTGTTGCAGTTAGTTACTTACTGGGTCCTCCCTGCCCAACTACAACCT
TTTCTTGAGCAGTGCCACAAGCCAAGAATGCCCCTGATTGTTAGCCGCAAGCACTCTCCA
GGACTTGATACTATTGTTGAATGAATGGCCGAATAGGAAATTTAACTTTCTGAAGGATTC
ACTTTTTTAAATTTCAGCTCAGCAAACACTGACCTGACTGTGTGCTACCAGAAGTTGATT
GGGCATATGGGTAGCTTCTGACTAATGGAATCACAATCAAAGCCCTGTCCTTCTTGCCCA
ACCTGACACTCCACACCCCACAGGGGCCTCACCTCCCTTCTTTCTGTCCCCCCATCTCTG
CTTCTACTGTTCCCTCTCCCAAGAGTGCTGTTCCTTCTGCTGCTTCCATCTAGTAAACTC
CTTTCGCTCTCCCTTTGGGGTCTCACCTTCTGTGGGAACTCAACTGCAGCTTTTGTCCGC
TGAACACATCCATTCATACATTCCTTCTCCATCCTGGGAAGATTTACTGAACACCTACTG
AGTGCCAGGTTCTGAGCATGCTGTTATCTCATTAGAACCTCACAGCATCCTAGGAGGGAG
GTGATCCTTCCAGCAGCCCCTCTCCCCAGGTCTCCATGTTCAGGATGTCCATGAGTTAGA
AATGAATATGTTCAGCTGCAAGTAACAGAAAAATCATAAAATAATGGGGGCTTAAGCAAG
ACAGAACTTTATTTTTTTCTCACCTAAAAGTTCAAGCTGGCAAATAGTCCAGGGACTCCT
TCCTGCCCACCTGGTTTGGGCATTGTCTGTCCCGATCTGGCTCCAAGACTCTCAGTCTTC
AACAAATCCTTTTGGCTGTCTATTAAAAATATTTACCAAATCCATCCTCTTCTCACCACC
TCCACTGCCAGCACTATCACCAGTCACCTAGACTACTGCAGTAGCCAGCTCCTTGCTGGT
CCCCTGCTTCCACATTCAGGCCCTAGACCCCATTGTACACTCAGCATTCAGGGGCTTCCT
GTTAAATCCTAACACGGTTTGTGTTTCTCCTCTGCTTGCAACCCTGTCATGCCTTCCTCC
TCACTCAGCGTTGAAGCCAAAGTCCTTACCATGGCCTACAAGGTCCTGCGTGGTCTAACC
TCCGAGTCCCCTTTCTGACCTCACCTCCAGCTTCTCTTCTTGCTCTCTCCACACTAACCA
CACCAGCCCCCTTGTAGACCTTGAACAAATCAGGTACATTCCCACCTCAGGACCTTTGCC
CTGGCTGTTTCCTTTGGAGTTCCTCAGATATCTAGCTGGCTCCCTCCCTCATCTCCTTCA
AGTCTTTGTCAGAGGCCCCTGTCTCTGGGGACTTTTCCTGACCCGTTTAATCTTGCCTAC
TCACCCCCAACTCCCAGCCCTCCCAGTCTCCTTCCCGAGAAGCCCTCCATGAGAGAGGAT
ATAGTCCAGTATAGAGGGGAGGTAGCGACCTCTACCCCTCTCTGCCCTCCTGACCCCAGC
CCCCATTCTGCAAGGATCTTGGCTTTCAGCAGCCGTAGCATTTCCCTCTCACACCCTGGG
CCTGCCCAAGGGGCCAGCTCACATTCTCGGCCACCATCCTCACTTCGTTTGCCTCCTATA
TCTCTGGCCACTCTCTTTCATGGAGTGAGTGACAGTAAGGGACAGATGGTCTGGGGAAGG
TATAAGGAGAATGCAGCCCACACCTCTGATCCTGAGAGAGAAAACTAGCCCCCTTTTCCA
GGAGGGGGACAGGTGCTGGGGCAGGGAACAATGCCTCTGCTGATAGAACTGGCCTGGTTT
ATAATTACGCAGGAGGTGTAGGGAACATTCCGACCCAAGTCTGCAGGGAGCAAGGGGTTA
TTAACTATGGGAACATCAGTGCAAGGGATCATTAACCTTAGGACCATCACTGCAAGGGGT
CATTAACCTTGGGGCCATCACTAGAATCAGCTCCCCAGGACTCCTTCCTGACATGCATGC
TCCTCACCCCCTCCCTTGCTCCCTCCCACATGCCGTGACATCTGTTAAACACCTGCTGTT
CACAGGGTCCCTGCAGGCCTCCAAGCAGGGGACCTCATCCCACCTTGGTGCCTCCCCTGG
CTGTCCAACACACCCAAGCCATTTTTGGGTCTCAGATGTGTCCCACCAACTCAGAAGCCT
CAACCAGCGAAAGTTTTGTTTTGAACTAATGGTGGGGAGGGTTGGGTGTTTTTTTTTTTT
TTTAATTGTTGTTGGCTGATTTTTTTAAGGTCTTTTTTTTTCTATTCTTTTTTTTTTTTT
TTCTTGTCTTCACAAAGCCAGGCCATCAGGCCCATTCTGGGCTTTTCCAGAAGGGTGAGT
CTGAGTTGAGGAAAGAGGCCCTCCTTTTTACGAGGGCTTAGAAGGTTGGGGGAGGAAAGG
GGGCAAAGGGGTGAAGGGGTGGATCTTGGCCACCAGATTTGTACCATGTAAATAAAAAGC
CCTTTCCAATTTCTCTTCCCCAGGTCTGAAGCCAGTCTTGTAGAGGGCTGGAGTGGTTGT
TGGACGACTAGAACCCTGGGCTTTGCAGGGTGCTGGGAGCTGGAAGTCTTCACCATGGCC
TGTCAGGAGAAGCCCCTGGGGGCTGGTCTACCATGCTGACCAGTGGTTGAATAGAGTGCC
TGCTGAGGTCAAGAGATGCAGCCGCCAATTCCTGGACAACCTAAAAGCCTCACAAGCTTC
AAGTCTCCAAGGACTCTCCAAATCCCAGGATCTCCCTGCCATCACATCCTAGAATTTAAA
ACCTGAGAACATCAAGTTCAGGAAAGCTTGGGGATTTCAAAGCCCAGCACCAAAGAAAGC
GGGACCCACACTCGCCAGCTCTGTGATCTTGGGCAAGTTGCTTGACTACTTATACCTCCG
TTTCCCTCATCTGTATAACAGGAATACAATAATGATGAGATGGAGCTCAAGGGGCCATGG
TGAGGATTGAACATGATCACTTGTGAGACATGTTCAGCACTGTATCTGACCCATGGAAAA
TTCAAGATAAACATCAGCTACTGAGATGATGGCGGATATTTGGAATCCTAAATCCTTGGA
AACTGGGGCTTTTTGAAGTAAAAGACCCCAAAGGCTGAGGGCCTCAGAAGCATCAGGCCA
TGATGTTCCTGAAACAAGAGGGTCAGGGTCCCAATGGGCCTCTGGGGTTCATCGTGAGGA
TGGATGCATTAATATTGGGGACCTGCTAGGGACCTTCCCAGTGGGACAGTGGCTGGGTCA
GGGCACTCAAGCCCTAAAACGTGATGAGGCGAGACTTTTCTCTCTTTCCTCATTCAGTAA
CTGTCAGTAGATTCTGGGAGCCAGGGATTCTCCGACTCTTCAAGTCCATGAATTTTAGGG
GATGACAGTGGGCTCTCCGCTTTCTCCTCCATGAAGTAACTTACATGCCCCTCACCCTCT
GTGGGAGGGGTGTTGCAGGGGGTGCAGAACTCCCCTCGCCGGGTAGTTCAAGCAATGGGG
ACCATATCAATTCCATCTATAGGGAAACTGAGGCCTGGAGTAGGGCGAGGCCTCTGGGAA
CCCAGCCCTATTCTGTCTCTTTCCCTGGCATTTCCCATCCACACATAGAGCTTCAGATTC
TCTTTCTTTCCCCAGAGACCCTCAAATATCCTCTCACTCACAGAATGGTGTCTCTGCCTG
CCTCGGGTTGGCCCTGTGATTTATTTTAGTTCTTTTCCCTTGTTTTTTTTTTTTCAAACT
CTATACACTTTTGTTTTAAAAACTGTGGTTTCTCATGAGCCCTATTATCTCATTGATACC
TCTCACCTCTGTGGTGAGGGGAAGAAATCATATTTTCAGATGACTCGTAAAGGGCAAAGA
AAAAAACCCAAAATTTCAAAATTTCCGTTTAAGTCTCATAATCAAGAAAAGGAGAAACAC
AGAGAGAGAGAAAAAAAAAACTATGAGAACCCCTCCCCACCCCGTGATTATCAGCGCACA
CACTCATCGAAAAAAATTTGGATTATTAGAAGAGAGAGGTCTGCGGCTTCCACACCGTAC
AGCGTGGTTTTTCTTCTCGGTATAAAAGCAAAGTTGTTTTTGATACGTGACAGTTTCCCA
CAAGCCAGGCTGATCCTTTTCTGTCAGTCCACTTCACCAAGGTGAGTGTCCCTGCTCTCC
CCTACCAGATGTGGGCCCCATTGGAGGAGATGGCAGGGAGGTAGGCACGGCGGGGGGGTC
AGGGGCCCTCTGGTACAGTGGGATGTACCCAGCTACCGTGATTCCAGCCAGGTAAGGTCT
TTAAAAAAATAATAGAATAAATGGCAGAAGACTTAAAGGTGAAGTACTTAATGCATGGGC
AGGGGCAAAAATAAATGAATTCATCAATTGATACGTAGATGGATAGGATTTTTAAAATAT
GTCAGCTGCCAGTCTTTATGGATTTCAGAGCTTTGAACAACTTGAAATGCTGTCCAGGTG
CAACTGAAAATTTCAACTGCATCTCTCTCATCTCTTTGTCTCTTAGCTCTTGATGCCTCT
CATCCTTCTCTGTCTCTCTGCTCACCCTGTCTTGCTCTTTTACATGTTTCTCTGCCTCTC
TTTCTCCCGCCATCTGTCTGTCGCTACACTCAGACTCTCTTTCCCCGCATCTCTCCTGGC
CTCTGACCATCTCCCCTATCTCTGTCTCCTCCATCTTGACCACACTCCATCGATCTCCTT
TCCTCCATCTCTCCCGCTCTCTTTTCTTGCCTTCCCCCACCTGCCTTCCCAGCCAGCATC
CAGCCTAGAGCTTTGTACACAGCTGGCGTTTAATAATAGTAATGTCGCCGAGGACCTTTA
CATTTTATGAGAGTGAGCATCAGTTCTGTTCACCCTAGTAGCAGGAGGATTGCAAGGTGC
CGGGGCAAGCTGAGCACTTAACTGAACTTTGCTTGTATCGCTTTAGAGGAGACCCTTGGG
GCGGGGGGGCAGGGAGCACTTCACACCATTGTCAACCGTAGTTTAGCTGTGTGGACCTAC
CCCAGGACTGCATGTGTGTGACCAGAGTCCATATTCAGGAAGCAAACAGGTTGTTCAGGG
CTGGGGTGAGCTCGATTCTGCAGGCTTAGCACAGCGTTTGGCACAAAGTAAGTGCTCACG
AAGTGTTAGCTATTAGTATTTCTGTAACCAAATTAGAATCATGCTATATATTGGTTTTGT
TCCTGCTTTATTCAATTAACAGTAGTGTTTTTGAAGCTTACTAAAAATTGTTTGAACACA
TGATCTTTCATGATGGACAAGACTGTCCTCACATCAGCATGAACTTTGATTGATTTGACC
AAGTGCCGATTGTTGGACATTCCGGTTGTTTCTCGTTTTTTTCTATAAACATCCCTGACT
GCTTCCTTGGATTCCTGAAAAGGGAGTTGCTGGGGCAAAGGGGGCGTGCATTTTTAAAGT
TTATGACCCTCTCTGCTAATTCTCCTTCCAAAAGGTTGTCCTATTTTATGCTCCCTCTAC
TTAAGCTAACCCTAGGTGAGTATTATTATGTTTTAATTATTTCCCAACATGACAGGTAAG
AAAAACACATTATCACATTGTTGAAATTTGCATCTTGTGATTACTTTTACTATTCATTGG
CCATTCATGCTTTTTTTTTTTTTTTTTTGGTTGTCTGGTCATGTCCTTACTCCTAATAGG
GTGTTCATCTTATTTTTCCCTTATTGATTTGCCACAGCTCTTTATTTGGAATGGTTATTA
ACCTTTCATCAGCCATTACATATATAGCAAATATTTTCCCCAATTCATCATTTGCTTTTT
TTCTATTTGTTTATGGTGTTTTTTGTGATGCTTATGATGGTTTTATACAGTCAAATAGGT
TAGTCTTTTTTTCTGTGGCTTCTGTCTCTGGTTTTGTGCTTAGAAAGTCCTTTCCTACTT
GAAAATGAGATAAATGTTCACCTATGTTGGCTTCTA
FLICR full sequence
>chromosome: GRCh38: X: 49262866: 49270521
(SEQ ID NO: 14)
GGATGGGATCTCCTGCTCCTGTTCCTCCCGCTCCTGCCCACGGCGGGCTGCGCGGGACGT
AGCGCGCTGGGTGGTCGCAAAAGGAAGCCCAGCCGTCGTGTGAGGCCCGCACGTGCACCG
CCTTCTCGAAGGAGCGGTTCAGCACGCGTACCAACCCGCGCAGCACCGGCGGGCGGCCCC
CAGGCACCCACACCCCGGCACCCCCGGGGACCGCTCCGGGAGGCGGCAGCAGCGCCTCCA
GCTCCACCATGACGCGCCCCAAGCGCTCCAGACGGCCCGGCGCGGGCGGCAGCGAAAATG
TGGGGACCAGGTAAAACCCCCCGCCAGCGGGGACGGGGCACAGCGGTGAGGGCTCGGGGC
AAGCCTCCTCTTCCTCCTCCCCTTCATCCCCATCCTCGCCATCGTCGTCCTCGTCGGCCC
CGCCGCCGCCGCCGCCATCTTGCCCGGCCGCCGCCGCCATCTTGCCCGCCCCGGGCCCGC
CCCACGGCCGGTAGCGGCGCAACTGCGCCAGCGGCAGCCCCAAGGCCTCGTCGGCGAACA
GCACCCTCCGCGGGGCCACCGCCGCCTCGACCGAGGTGCGGGGCTCACCCGCGGCCGGCG
AGGGGGGCGCGGAATGCCGCAGCGGGGGCTCCACAGGGGCCGTACGCGCCATATCGGCGG
CGGCGGCGGCACCGACGGCACCGGCGGCGGGACCGGCGGGGGCAGGGCCTGAAGGGGCGG
GCAGCCAATGGGAAGGCCAGCGCGGAGGGGTACGGCTCACGGGGGCATAGGCGGTGAGAT
GAAGACAAGTATTGCAGAGGGCCAATGGGCAGCCATGCACGGATGACGTACAGCGAATTG
AGAGCCGGAGAAGCACGCAGCCAATGGGGATGCCCGGGAGTGGGCGGTGTAGGGGGCGGG
ATGATCGAGGAATCAATGGAGAGGCCGGAATGGGGGTGGTGCGAAATCCATGATGAAGAA
AACAGAAGCAGACAGCTAATGCGGAAGTCTGAGCGAGAGAGGCATGTGGGCGGTGGGAAA
AGTGGAATGGCTACAGGCGATGAGGGCAGCCGGGAACGAACGCAGAGGCCCAAAAAGTGG
GAGATCCCGCGGGTTGCGTAGGCGGTGGAACGGTAGCTAGAGAGAGAGAACAGCCAATGG
AGAGCCACGCGTGGGATGACGACAATGGCTGCAGCGAGCCAATGGGGAGGCTTGTGAGCG
TGCACTGCGGCTTCGAAGGGGTGGTGCCAAGGTGGAGCTAGCCAATACGAAGGCTAAAAG
GAGACCAAGAGGCGGGTGTGTAGGGAGCCGAGCCAATGGAGGTGGAGGCTGAAGGCGGGA
GCGAGGACGAGAGCTGGCGGATAGCCAATGAGGGAGCCAGGGTGAAGGCGTAGAGACCTC
AAAGGACAGTACTAGTATGGGATGAGCCAATGCCGAGGCTAGATAGTGCAGCGGGACGGA
GGGAGGACTTGGAGGCAAGATGGGGAAAAAAGCAGAGCCGACGGGGGCAGTGTAGGTGGT
GTGAAGGGAGGCATGAGTAGCCAATAAGGAGGCTGGAGAGGAGGACGCTGTGTAGACAGA
GTCTGAGGTATTGGGTGGCCGGACAGCAGAACCATAGAACGCAGGCAGCCATTAGGAAAA
ATGTGCTTCTCAGGGATGGGGTTACCCTCTCCCTAACCCTCCGTGACTCCCCATTGCCCC
AGGAATACCCAGAGCCCCTGAAGGCCTAGACCACACCTTGGTGTGTGAACTAGGAAAGTG
AAAATGACTGTCCTGTTGCAGTTAGTTACTTACTGGGTCCTCCCTGCCCAACTACAACCT
TTTCTTGAGCAGTGCCACAAGCCAAGAATGCCCCTGATTGTTAGCCGCAAGCACTCTCCA
GGACTTGATACTATTGTTGAATGAATGGCCGAATAGGAAATTTAACTTTCTGAAGGATTC
ACTTTTTTAAATTTCAGCTCAGCAAACACTGACCTGACTGTGTGCTACCAGAAGTTGATT
GGGCATATGGGTAGCTTCTGACTAATGGAATCACAATCAAAGCCCTGTCCTTCTTGCCCA
ACCTGACACTCCACACCCCACAGGGGCCTCACCTCCCTTCTTTCTGTCCCCCCATCTCTG
CTTCTACTGTTCCCTCTCCCAAGAGTGCTGTTCCTTCTGCTGCTTCCATCTAGTAAACTC
CTTTCGCTCTCCCTTTGGGGTCTCACCTTCTGTGGGAACTCAACTGCAGCTTTTGTCCGC
TGAACACATCCATTCATACATTCCTTCTCCATCCTGGGAAGATTTACTGAACACCTACTG
AGTGCCAGGTTCTGAGCATGCTGTTATCTCATTAGAACCTCACAGCATCCTAGGAGGGAG
GTGATCCTTCCAGCAGCCCCTCTCCCCAGGTCTCCATGTTCAGGATGTCCATGAGTTAGA
AATGAATATGTTCAGCTGCAAGTAACAGAAAAATCATAAAATAATGGGGGCTTAAGCAAG
ACAGAACTTTATTTTTTTCTCACCTAAAAGTTCAAGCTGGCAAATAGTCCAGGGACTCCT
TCCTGCCCACCTGGTTTGGGCATTGTCTGTCCCGATCTGGCTCCAAGACTCTCAGTCTTC
AACAAATCCTTTTGGCTGTCTATTAAAAATATTTACCAAATCCATCCTCTTCTCACCACC
TCCACTGCCAGCACTATCACCAGTCACCTAGACTACTGCAGTAGCCAGCTCCTTGCTGGT
CCCCTGCTTCCACATTCAGGCCCTAGACCCCATTGTACACTCAGCATTCAGGGGCTTCCT
GTTAAATCCTAACACGGTTTGTGTTTCTCCTCTGCTTGCAACCCTGTCATGCCTTCCTCC
TCACTCAGCGTTGAAGCCAAAGTCCTTACCATGGCCTACAAGGTCCTGCGTGGTCTAACC
TCCGAGTCCCCTTTCTGACCTCACCTCCAGCTTCTCTTCTTGCTCTCTCCACACTAACCA
CACCAGCCCCCTTGTAGACCTTGAACAAATCAGGTACATTCCCACCTCAGGACCTTTGCC
CTGGCTGTTTCCTTTGGAGTTCCTCAGATATCTAGCTGGCTCCCTCCCTCATCTCCTTCA
AGTCTTTGTCAGAGGCCCCTGTCTCTGGGGACTTTTCCTGACCCGTTTAATCTTGCCTAC
TCACCCCCAACTCCCAGCCCTCCCAGTCTCCTTCCCGAGAAGCCCTCCATGAGAGAGGAT
ATAGTCCAGTATAGAGGGGAGGTAGCGACCTCTACCCCTCTCTGCCCTCCTGACCCCAGC
CCCCATTCTGCAAGGATCTTGGCTTTCAGCAGCCGTAGCATTTCCCTCTCACACCCTGGG
CCTGCCCAAGGGGCCAGCTCACATTCTCGGCCACCATCCTCACTTCGTTTGCCTCCTATA
TCTCTGGCCACTCTCTTTCATGGAGTGAGTGACAGTAAGGGACAGATGGTCTGGGGAAGG
TATAAGGAGAATGCAGCCCACACCTCTGATCCTGAGAGAGAAAACTAGCCCCCTTTTCCA
GGAGGGGGACAGGTGCTGGGGCAGGGAACAATGCCTCTGCTGATAGAACTGGCCTGGTTT
ATAATTACGCAGGAGGTGTAGGGAACATTCCGACCCAAGTCTGCAGGGAGCAAGGGGTTA
TTAACTATGGGAACATCAGTGCAAGGGATCATTAACCTTAGGACCATCACTGCAAGGGGT
CATTAACCTTGGGGCCATCACTAGAATCAGCTCCCCAGGACTCCTTCCTGACATGCATGC
TCCTCACCCCCTCCCTTGCTCCCTCCCACATGCCGTGACATCTGTTAAACACCTGCTGTT
CACAGGGTCCCTGCAGGCCTCCAAGCAGGGGACCTCATCCCACCTTGGTGCCTCCCCTGG
CTGTCCAACACACCCAAGCCATTTTTGGGTCTCAGATGTGTCCCACCAACTCAGAAGCCT
CAACCAGCGAAAGTTTTGTTTTGAACTAATGGTGGGGAGGGTTGGGTGTTTTTTTTTTTT
TTTAATTGTTGTTGGCTGATTTTTTTAAGGTCTTTTTTTTTCTATTCTTTTTTTTTTTTT
TTCTTGTCTTCACAAAGCCAGGCCATCAGGCCCATTCTGGGCTTTTCCAGAAGGGTGAGT
CTGAGTTGAGGAAAGAGGCCCTCCTTTTTACGAGGGCTTAGAAGGTTGGGGGAGGAAAGG
GGGCAAAGGGGTGAAGGGGTGGATCTTGGCCACCAGATTTGTACCATGTAAATAAAAAGC
CCTTTCCAATTTCTCTTCCCCAGGTCTGAAGCCAGTCTTGTAGAGGGCTGGAGTGGTTGT
TGGACGACTAGAACCCTGGGCTTTGCAGGGTGCTGGGAGCTGGAAGTCTTCACCATGGCC
TGTCAGGAGAAGCCCCTGGGGGCTGGTCTACCATGCTGACCAGTGGTTGAATAGAGTGCC
TGCTGAGGTCAAGAGATGCAGCCGCCAATTCCTGGACAACCTAAAAGCCTCACAAGCTTC
AAGTCTCCAAGGACTCTCCAAATCCCAGGATCTCCCTGCCATCACATCCTAGAATTTAAA
ACCTGAGAACATCAAGTTCAGGAAAGCTTGGGGATTTCAAAGCCCAGCACCAAAGAAAGC
GGGACCCACACTCGCCAGCTCTGTGATCTTGGGCAAGTTGCTTGACTACTTATACCTCCG
TTTCCCTCATCTGTATAACAGGAATACAATAATGATGAGATGGAGCTCAAGGGGCCATGG
TGAGGATTGAACATGATCACTTGTGAGACATGTTCAGCACTGTATCTGACCCATGGAAAA
TTCAAGATAAACATCAGCTACTGAGATGATGGCGGATATTTGGAATCCTAAATCCTTGGA
AACTGGGGCTTTTTGAAGTAAAAGACCCCAAAGGCTGAGGGCCTCAGAAGCATCAGGCCA
TGATGTTCCTGAAACAAGAGGGTCAGGGTCCCAATGGGCCTCTGGGGTTCATCGTGAGGA
TGGATGCATTAATATTGGGGACCTGCTAGGGACCTTCCCAGTGGGACAGTGGCTGGGTCA
GGGCACTCAAGCCCTAAAACGTGATGAGGCGAGACTTTTCTCTCTTTCCTCATTCAGTAA
CTGTCAGTAGATTCTGGGAGCCAGGGATTCTCCGACTCTTCAAGTCCATGAATTTTAGGG
GATGACAGTGGGCTCTCCGCTTTCTCCTCCATGAAGTAACTTACATGCCCCTCACCCTCT
GTGGGAGGGGTGTTGCAGGGGGTGCAGAACTCCCCTCGCCGGGTAGTTCAAGCAATGGGG
ACCATATCAATTCCATCTATAGGGAAACTGAGGCCTGGAGTAGGGCGAGGCCTCTGGGAA
CCCAGCCCTATTCTGTCTCTTTCCCTGGCATTTCCCATCCACACATAGAGCTTCAGATTC
TCTTTCTTTCCCCAGAGACCCTCAAATATCCTCTCACTCACAGAATGGTGTCTCTGCCTG
CCTCGGGTTGGCCCTGTGATTTATTTTAGTTCTTTTCCCTTGTTTTTTTTTTTTCAAACT
CTATACACTTTTGTTTTAAAAACTGTGGTTTCTCATGAGCCCTATTATCTCATTGATACC
TCTCACCTCTGTGGTGAGGGGAAGAAATCATATTTTCAGATGACTCGTAAAGGGCAAAGA
AAAAAACCCAAAATTTCAAAATTTCCGTTTAAGTCTCATAATCAAGAAAAGGAGAAACAC
AGAGAGAGAGAAAAAAAAAACTATGAGAACCCCTCCCCACCCCGTGATTATCAGCGCACA
CACTCATCGAAAAAAATTTGGATTATTAGAAGAGAGAGGTCTGCGGCTTCCACACCGTAC
AGCGTGGTTTTTCTTCTCGGTATAAAAGCAAAGTTGTTTTTGATACGTGACAGTTTCCCA
CAAGCCAGGCTGATCCTTTTCTGTCAGTCCACTTCACCAAGGTGAGTGTCCCTGCTCTCC
CCTACCAGATGTGGGCCCCATTGGAGGAGATGGCAGGGAGGTAGGCACGGCGGGGGGGTC
AGGGGCCCTCTGGTACAGTGGGATGTACCCAGCTACCGTGATTCCAGCCAGGTAAGGTCT
TTAAAAAAATAATAGAATAAATGGCAGAAGACTTAAAGGTGAAGTACTTAATGCATGGGC
AGGGGCAAAAATAAATGAATTCATCAATTGATACGTAGATGGATAGGATTTTTAAAATAT
GTCAGCTGCCAGTCTTTATGGATTTCAGAGCTTTGAACAACTTGAAATGCTGTCCAGGTG
CAACTGAAAATTTCAACTGCATCTCTCTCATCTCTTTGTCTCTTAGCTCTTGATGCCTCT
CATCCTTCTCTGTCTCTCTGCTCACCCTGTCTTGCTCTTTTACATGTTTCTCTGCCTCTC
TTTCTCCCGCCATCTGTCTGTCGCTACACTCAGACTCTCTTTCCCCGCATCTCTCCTGGC
CTCTGACCATCTCCCCTATCTCTGTCTCCTCCATCTTGACCACACTCCATCGATCTCCTT
TCCTCCATCTCTCCCGCTCTCTTTTCTTGCCTTCCCCCACCTGCCTTCCCAGCCAGCATC
CAGCCTAGAGCTTTGTACACAGCTGGCGTTTAATAATAGTAATGTCGCCGAGGACCTTTA
CATTTTATGAGAGTGAGCATCAGTTCTGTTCACCCTAGTAGCAGGAGGATTGCAAGGTGC
CGGGGCAAGCTGAGCACTTAACTGAACTTTGCTTGTATCGCTTTAGAGGAGACCCTTGGG
GCGGGGGGGCAGGGAGCACTTCACACCATTGTCAACCGTAGTTTAGCTGTGTGGACCTAC
CCCAGGACTGCATGTGTGTGACCAGAGTCCATATTCAGGAAGCAAACAGGTTGTTCAGGG
CTGGGGTGAGCTCGATTCTGCAGGCTTAGCACAGCGTTTGGCACAAAGTAAGTGCTCACG
AAGTGTTAGCTATTAGTATTTCTGTAACCAAATTAGAATCATGCTATATATTGGTTTTGT
TCCTGCTTTATTCAATTAACAGTAGTGTTTTTGAAGCTTACTAAAAATTGTTTGAACACA
TGATCTTTCATGATGGACAAGACTGTCCTCACATCAGCATGAACTTTGATTGATTTGACC
AAGTGCCGATTGTTGGACATTCCGGTTGTTTCTCGTTTTTTTCTATAAACATCCCTGACT
GCTTCCTTGGATTCCTGAAAAGGGAGTTGCTGGGGCAAAGGGGGCGTGCATTTTTAAAGT
TTATGACCCTCTCTGCTAATTCTCCTTCCAAAAGGTTGTCCTATTTTATGCTCCCTCTAC
TTAAGCTAACCCTAGGTGAGTATTATTATGTTTTAATTATTTCCCAACATGACAGGTAAG
AAAAACACATTATCACATTGTTGAAATTTGCATCTTGTGATTACTTTTACTATTCATTGG
CCATTCATGCTTTTTTTTTTTTTTTTTTGGTTGTCTGGTCATGTCCTTACTCCTAATAGG
GTGTTCATCTTATTTTTCCCTTATTGATTTGCCACAGCTCTTTATTTGGAATGGTTATTA
ACCTTTCATCAGCCATTACATATATAGCAAATATTTTCCCCAATTCATCATTTGCTTTTT
TTCTATTTGTTTATGGTGTTTTTTGTGATGCTTATGATGGTTTTATACAGTCAAATAGGT
TAGTCTTTTTTTCTGTGGCTTCTGTCTCTGGTTTTGTGCTTAGAAAGTCCTTTCCTACTT
GAAAATGAGATAAATGTTCACCTATGTTGGCTTCTA

### BIBLIOGRAPHY

Dikiy et al., Immunity (2021)
Ferreira et al., Nat. Rev. Drug Discov (2019)
Lu et al., Nat Rev Immunol. (2017)
Lucca et al., Nat Rev Immunol. (2020)
Sanson, K. R., et al., Nat. Commun (2018)
Schmidt, R & Steinhart, Z et al. Science (2022)
Shifrut, E & Carnevale, J et al. Cell (2018)
Schumann et al., Nat Immunol (2020
Zemmour et al., PNAS (2017)
Zheng et al., Nature (2010)

All publications, patent applications, patents and other references mentioned herein are expressly incorporated by reference in their entirety, to the same extent as if each were incorporated by reference individually. In case of conflict, the present specification, including definitions, will control.

The following statements provide a summary of some aspects of the inventive nucleic acids and methods described herein.

### Statements:

1. A method to identify regulators of Treg cells, comprising:
contacting human Treg cells with one or more gRNAs targeted to one or more coding or non-coding regions in one or more genes and a polypeptide comprising a Cas polypeptide or nucleic acid encoding the polypeptide; and
selecting one or more human Treg cells that have an altered activity and optionally isolating the Treg cells with altered activity.

2. The method of statement 1 wherein the gRNAs are targeted to FOXP3, MED12, FOXP1, ATXN7L3, GATA3, USP22, MED30, RUNX3, CBFB, MED14, MED11, STAT5A, NFKB2, IL2RA, IRF4, HSF2, RELA, ZMYND8, CTCF, HIC1, TGFBR1, or TBX21.

3. The method of statement 1 wherein the gRNAs are targeted to MTF2, TUBB, EGR2, SATB1, YBX1, ZNF740, EGR3, SRF, or PTEN.

4. The method of statement 1 wherein the gRNAs are targeted to RUNX3, CBFB, DNMT1, E2F3, EGR2, EGR3, ETS1, FOXO1, GABPA, GATA3, HIC1, HIF1A, HSF2, FOXN2, IL2RA, IRF2, IRF4, SMAD4, MTF1, NFKB2, YBX1, MAP2K1, PTEN, RELA, SATB1, SRF, STAT5A, STAT5B, TFDP1, TGFBR1, YY1, ZNF143, NR4A3, MBD2, MED14, SETDB1, MED12, IKZF1, CTCF, GMEB1, IKZF3, MTF2, MGA, USP22, ZMYND8, ZBTB32, FOXP1, TAF5L, TBX21, FOXP3, ATXN7L3, VARS2, ZNF574, BCL11B, MED30, TUBB, ZNF740, or MED11.

5. The method of statement 1 wherein the gRNAs are targeted to any one of SEQ ID Nos. 20-23.

6. The method of statement 1 wherein the gRNAs comprise one or more of SEQ ID NOs: 102-379 or 402-424, including SEQ ID NOs: 102-109, 144, 146, 148, 150, 152-155, 160-163, 168-171, 176, 178, 180, 182, 192, 194, 196, 198, 220-223, 236, 238, 240, 242, 252-255, 272-275, 280-283, 288-291, 308-315, 320, 322, 324, 326, 332-336, 338, 340, 342, 344, 346, 348, 350, 364-367 or 376-379, or a sequence with at least 80%, 82%, 85%, 87%, 90%, 92%, 94%, 95%, 97%, 98% or 99% nucleic acid sequence identity thereto.

7. The method of statement 1 wherein the gRNAs comprise one or more of SEQ ID Nos. 110-113, 115-125, 127-135, 164-167, 172-175, 188-191, 200, 202-212, 214, 216, 218, 224-235, 248-251, 268-271, 292-295, 300-303, 352-359 or 368-375 or a sequence with at least 80%, 82%, 85%, 87%, 90%, 92%, 94%, 95%, 97%, 98% or 99% nucleic acid sequence identity thereto.

8. The method of statement 1 wherein the gRNAs comprise one or more of SEQ ID Nos. 402-407 or a sequence with at least 80%, 82%, 85%, 87%, 90%, 92%, 94%, 95%, 97%, 98% or 99% nucleic acid sequence identity thereto.

9. The method of statement 1 wherein the gRNAs comprise one or more of SEQ ID Nos. 408-413 or a sequence with at least 80%, 82%, 85%, 87%, 90%, 92%, 94%, 95%, 97%, 98% or 99% nucleic acid sequence identity thereto.

10. The method of statement 1 wherein the Tregs are from a human with cancer or wherein the Tregs are from a human receiving transplanted tissue(s)/organ(s)/cells.

11. The method of statement 1 wherein the Tregs are from a human with an autoimmune disease.

11A. The method of statement 1 wherein the Tregs are from a donor transferred to a human with cancer, autoimmune disease or transplant patient.

12. An isolated nucleic acid comprising one of SEQ ID Nos. 402-424 or a combination thereof.

13. A vector comprising at least one of SEQ ID Nos. 402-424.

14. A complex comprising a Cas polypeptide and one of SEQ ID Nos. 402-424.

15. An isolated human Treg having altered FOXP3 activity as a result of a modification associated with a gRNA having one of SEQ ID Nos. 102-109, 144, 146, 148, 150, 152-155, 160-163, 168-171, 176, 178, 180, 182, 192, 194, 196, 198, 220-223, 236, 238, 240, 242, 252-255, 272-275, 280-283, 288-291, 308-315, 320, 322, 324, 326, 332-336, 338, 340, 342, 344, 346, 348, 350, 364-367, 376-379, 110-113, 115-125, 127-135, 164-167, 172-175, 188-191, 200, 202-212, 214, 216, 218, 224-235, 248-251, 268-271, 292-295, 300-303, 352-359, 368-375, or 402-413, or a combination thereof

16. An isolated human Treg having altered FOXP3 activity as a result of a genetic modification in one of RUNX3, CBFB, DNMT1, E2F3, EGR2, EGR3, ETS1, FOXO1, GABPA, GATA3, HIC1, HIF1A, HSF2, FOXN2, IL2RA, IRF2, IRF4, SMAD4, MTF1, NFKB2, YBX1, MAP2K1, PTEN, RELA, SATB1, SRF, STAT5A, STAT5B, TFDP1, TGFBR1, YY1, ZNF143, NR4A3, MBD2, MED14, SETDB1, MED12, IKZF1, CTCF, GMEB1, IKZF3, MTF2, MGA, USP22, ZMYND8, ZBTB32, FOXP1, TAF5L, TBX21, FOXP3, ATXN7L3, VARS2, ZNF574, BCL11B, MED30, TUBB, ZNF740, or MED11.

17. An isolated human Treg having altered FOXP3 activity as a result of a genetic modification in any one of SEQ ID Nos. 20-23.

18. A method of modifying Treg activity, comprising: providing isolated Tregs from a human and contacting the Tregs with one or more of SEQ ID Nos. 102-379 or 402-424.

19. The method of statement 18 wherein the human has cancer.

20. The method of statement 18 wherein the human has an autoimmune disease.

21. The method of statement 18 wherein the human has had an organ tissue or cell transplant.

22. A method to prevent, inhibit or treat cancer in a mammal, comprising administering to the mammal a composition having a plurality of human Treg cells modified with one of SEQ ID NOs: 102-109, 144, 146, 148, 150, 152-155, 160-163, 168-171, 176, 178, 180, 182, 192, 194, 196, 198, 220-223, 236, 238, 240, 242, 252-255, 272-275, 280-283, 288-291, 308-315, 320, 322, 324, 326, 332-336, 338, 340, 342, 344, 346, 348, 350, 364-367 or 376-379, or 408-413.

23. A method to prevent, inhibit or treat cancer in a mammal, comprising: providing human Treg cells from a mammal modified with one of SEQ ID NOs: 102-109, 144, 146, 148, 150, 152-155, 160-163, 168-171, 176, 178, 180, 182, 192, 194, 196, 198, 220-223, 236, 238, 240, 242, 252-255, 272-275, 280-283, 288-291, 308-315, 320, 322, 324, 326, 332-336, 338, 340, 342, 344, 346, 348, 350, 364-367 or 376-379, or 408-413 and administering the modified human Treg cells to the mammal.

24. A method to prevent, inhibit or treat an autoimmune disease in a mammal, comprising administering to the mammal a composition having a plurality of Treg cells modified with one of SEQ ID NOs: 110-113, 115-125, 127-135, 164-167, 172-175, 188-191, 200, 202-212, 214, 216, 218, 224-235, 248-251, 268-271, 292-295, 300-303, 352-359 or 368-375, or 402-407.

25. A method to prevent, inhibit or treat an autoimmune disease in a mammal, comprising: providing human Treg cells from a mammal modified with one of SEQ ID NOs: 110-113, 115-125, 127-135, 164-167, 172-175, 188-191, 200, 202-212, 214, 216, 218, 224-235, 248-251, 268-271, 292-295, 300-303, 352-359 or 368-375, or 402-407 and administering the modified human Treg cells to the mammal.

1. An isolated human Treg cell having altered FOXP3 activity as a result of a modification associated with a gRNA having one of SEQ ID NOs: 102-379 and 402-424, including, 102-109, 144, 146, 148, 150, 152-155, 160-163, 168-171, 176, 178, 180, 182, 192, 194, 196, 198, 220-223, 236, 238, 240, 242, 252-255, 272-275, 280-283, 288-291, 308-315, 320, 322, 324, 326, 332-336, 338, 340, 342, 344, 346, 348, 350, 364-367 or 376-379, SEQ ID NOs: 110-113, 115-125, 127-135, 164-167, 172-175, 188-191, 200, 202-212, 214, 216, 218, 224-235, 248-251, 268-271, 292-295, 300-303, 352-359 or 368-375; or SEQ ID NOs: 402-413, or a combination thereof.

2. An isolated human Treg cell having altered FOXP3 activity as a result of a genetic modification in one of RUNX3, CBFB, DNMT1, E2F3, EGR2, EGR3, ETS1, FOXO1, GABPA, GATA3, HIC1, HIF1A, HSF2, FOXN2, IL2RA, IRF2, IRF4, SMAD4, MTF1, NFKB2, YBX1, MAP2K1, PTEN, RELA, SATB1, SRF, STAT5A, STAT5B, TFDP1, TGFBR1, YY1, ZNF143, NR4A3, MBD2, MED14, SETDB1, MED12, IKZF1, CTCF, GMEB1, IKZF3, MTF2, MGA, USP22, ZMYND8, ZBTB32, FOXP1, TAF5L, TBX21, FOXP3, ATXN7L3, VARS2, ZNF574, BCL11B, MED30, TUBB, ZNF740 or MED11.

3. An isolated human Treg cell having altered FOXP3 activity as a result of a genetic modification in any one of SEQ ID NOs: 20-23.

4. An isolated human Treg having altered FOXP3 activity as a result of a genetic modification in one of RUNX3, CBFB, GATA3, HIC1, HSF2, IL2RA, IRF4, NFKB2, RELA, STAT5A, TGFBR1, MED14, MED12, CTCF, USP22, ZMYND8, FOXP1, TBX21, FOXP3, ATXN7L3, MED30, or MED11.

5. An isolated human Treg cell having altered FOXP3 activity as a result of a genetic modification in one of EGR2, EGR3, YBX1, PTEN, RELA, SATB1, SRF, MTF2, TUBB, or ZNF740.

6. A method to prevent, inhibit or treat cancer in a mammal, comprising administering to the mammal a composition having a plurality of human Treg cells modified with one or more of SEQ ID NOs: 102-109, 144, 146, 148, 150, 152-155, 160-163, 168-171, 176, 178, 180, 182, 192, 194, 196, 198, 220-223, 236, 238, 240, 242, 252-255, 272-275, 280-283, 288-291, 308-315, 320, 322, 324, 326, 332-336, 338, 340, 342, 344, 346, 348, 350, 364-367 or 376-379 or 408-413.

7. A method to prevent, inhibit or treat cancer in a mammal, comprising administering to the mammal a composition having a plurality of human Treg cells having a genetic modification in one or more of RUNX3, CBFB, DNMT1, E2F3, EGR2, EGR3, ETS1, FOXO1, GABPA, GATA3, HIC1, HIF1A, HSF2, FOXN2, IL2RA, IRF2, IRF4, SMAD4, MTF1, NFKB2, YBX1, MAP2K1, PTEN, RELA, SATB1, SRF, STAT5A, STAT5B, TFDP1, TGFBR1, YY1, ZNF143, NR4A3, MBD2, MED14, SETDB1, MED12, IKZF1, CTCF, GMEB1, IKZF3, MTF2, MGA, USP22, ZMYND8, ZBTB32, FOXP1, TAF5L, TBX21, FOXP3, ATXN7L3, VARS2, ZNF574, BCL11B, MED30, TUBB, ZNF740 or MED11.

8. A method to prevent, inhibit or treat cancer in a mammal, comprising administering to the mammal a composition having a plurality of human Treg cells having a genetic modification in one or more of SEQ ID NOs: 20 or 21.

9. The method of claim 6, wherein the modified cells have reduced Treg activity relative to unmodified cells.

10.-12. (canceled)

13. The method of any one-of claim 6, wherein the cancer is an immune cell cancer.

14. The method of any one-of claim 6, wherein the cancer is a carcinomas; a leukemia; a benign or malignant lymphoma; a benign or malignant melanoma; a myeloproliferative disease; a sarcoma; a tumor of the central nervous system; a germ-line tumor; mixed types of neoplasias; or tumors of mixed origin.

15. The method of claim 6, wherein the cells are autologous or allogeneic cells.

16. A method to prevent, inhibit or treat an autoimmune disease in a mammal, comprising administering to the mammal a composition having a plurality of Treg cells modified with one of SEQ ID NOs: 110-113, 115-125, 127-135, 164-167, 172-175, 188-191, 200, 202-212, 214, 216, 218, 224-235, 248-251, 268-271, 292-295, 300-303, 352-359 or 368-375 or 402-407.

17. A method to prevent, inhibit or treat an autoimmune disease in a mammal, comprising administering to the mammal a composition having a plurality of Treg cells with a genetic modification in RUNX3, CBFB, DNMT1, E2F3, EGR2, EGR3, ETS1, FOX01, GABPA, GATA3, HIC1, HIF1A, HSF2, FOXN2, IL2RA, IRF2, IRF4, SMAD4, MTF1, NFKB2, YBX1, MAP2K1, PTEN, RELA, SATB1, SRF, STAT5A, STAT5B, TFDP1, TGFBR1, YY1, ZNF143, NR4A3, MBD2, MED14, SETDB1, MED12, IKZF1, CTCF, GMEB1, IKZF3, MTF2, MGA, USP22, ZMYND8, ZBTB32, FOXP1, TAF5L, TBX21, FOXP3, ATXN7L3, VARS2, ZNF574, BCL11B, MED30, TUBB, ZNF740 or MED11.

18. A method to prevent, inhibit or treat an autoimmune disease in a mammal, comprising administering to the mammal a composition having a plurality of Treg cells having a genetic modification in one of SEQ ID NOs: 7-14.

19. The method of claim 16, where the modified cells have enhanced activity relative to unmodified cells.

20.-22. (canceled)

23. The method of claim 16, wherein the autoimmune disease is rheumatoid arthritis, Crohn's disease, multiple sclerosis (MS), systemic sclerosis (SSc), systemic lupus erythematosus (SLE), autoimmune encephalomyelitis, myasthenia gravis (MG), Hashimoto's thyroiditis, Goodpasture's syndrome, pemphigus (e.g., pemphigus vulgaris), Grave's disease (GD), aplastic anemia (AA), vitiligo, autoimmune hemolytic anemia, autoimmune thrombocytopenia purpura, scleroderma with anti-collagen antibodies, mixed connective tissue disease, polymyositis, pernicious anemia, idiopathic Addison's disease, autoimmune-associated infertility, glomerulonephritis (e.g., crescentic glomerulonephritis, proliferative glomerulonephritis), graft vs host disease (GvHD) or promote organ transplant tolerance, bullous pemphigoid, Sjogren's syndrome, insulin resistance, or autoimmune diabetes mellitus (type 1 diabetes (TID) mellitus; insulin-dependent diabetes mellitus).

24. The method of claim 16, wherein the cells are autologous cells or allogeneic cells.

25. A method to identify regulators of Treg cells, comprising:
contacting human Treg cells with one or more gRNAs targeted to one or more coding or non-coding regions in one or more genes and a polypeptide comprising a Cas polypeptide or nucleic acid encoding the polypeptide; and
selecting one or more human Treg cells that have an altered activity and optionally isolating the Treg cells with altered activity.

26. The method of claim 25, wherein the human Treg cells are contacted with a library of gRNAs targeted to the one or more coding regions in a plurality of genes.

27. The method of claim 25, wherein a library of viruses expresses the one or more gRNAs.

28. The method of claim 27, wherein the virus is a lentivirus, retrovirus, adenovirus, herpesvirus or adeno-associated virus.

29. The method of claim 25, further comprising stimulating T cell receptors prior to selecting.

30. The method of claim 25, wherein T regs are selected that have decreased activity.

31. The method of claim 25, wherein T regs are selected that have increased activity.

32. The method of claim 25, wherein the Cas enzyme is fused to a transcriptional activator, transcriptional repressor, base editor, or other epigenetic machinery.

33. The method of claim 25, wherein the Tregs have increased FOXP3 activity.

34. The method of claim 25, wherein the Tregs have decreased FOXP3 activity.

35. The method of claim 25, wherein the gRNAs are targeted to FOXP3, MED12, FOXP1, ATXN7L3, GATA3, USP22, MED30, RUNX3, CBFB, MED14, MED11, STAT5A, NFKB2, IL2RA, IRF4, HSF2, RELA, ZMYND8, CTCF, HIC1, TGFBR1, or TBX21.

36. The method of claim 25, wherein the gRNAs are targeted to MTF2, TUBB, EGR2, SATB1, YBX1, ZNF740, EGR3, SRF or PTEN.

37. The method of claim 25, wherein the gRNAs are targeted to RUNX3, CBFB, DNMT1, E2F3, EGR2, EGR3, ETS1, FOXO1, GABPA, GATA3, HIC1, HIF1A, HSF2, FOXN2, IL2RA, IRF2, IRF4, SMAD4, MTF1, NFKB2, YBX1, MAP2K1, PTEN, RELA, SATB1, SRF, STAT5A, STAT5B, TFDP1, TGFBR1, YY1, ZNF143, NR4A3, MBD2, MED14, SETDB1, MED12, IKZF1, CTCF, GMEB1, IKZF3, MTF2, MGA, USP22, ZMYND8, ZBTB32, FOXP1, TAF5L, TBX21, FOXP3, ATXN7L3, VARS2, ZNF574, BCL11B, MED30, TUBB, ZNF740, or MED11.

38. The method of claim 25, wherein the gRNAs are targeted to RUNX3, CBFB, GATA3, HIC1, HSF2, IL2RA, IRF4, NFKB2, RELA, STAT5A, TGFBR1, MED14, MED12, CTCF, USP22, ZMYND8, FOXP1, TBX21, FOXP3, ATXN7L3, MED30, or MED11

39. The method of claim 25, wherein the gRNAs are targeted to EGR2, EGR3, YBX1, PTEN, RELA, SATB1, SRF, MTF2, TUBB, or ZNF740

40. The method of claim 25, wherein the gRNAs are targeted to any one of SEQ ID NOs: 20-23.

41. The method of claim 25, wherein the gRNAs comprise one or more of SEQ ID NOs: 102-109, 144, 146, 148, 150, 152-155, 160-163, 168-171, 176, 178, 180, 182, 192, 194, 196, 198, 220-223, 236, 238, 240, 242, 252-255, 272-275, 280-283, 288-291, 308-315, 320, 322, 324, 326, 332-336, 338, 340, 342, 344, 346, 348, 350, 364-367 or 376-379, or a sequence with at least 80%, 82%, 85%, 87%, 90%, 92%, 94%, 95%, 97%, 98% or 99% nucleic acid sequence identity thereto.

42. The method of claim 25, wherein the gRNAs comprise one or more of SEQ ID NOs: 110-113, 115-125, 127-135, 164-167, 172-175, 188-191, 200, 202-212, 214, 216, 218, 224-235, 248-251, 268-271, 292-295, 300-303, 352-359 or 368-375 or a sequence with at least 80%, 82%, 85%, 87%, 90%, 92%, 94%, 95%, 97%, 98% or 99% nucleic acid sequence identity thereto.

43. The method of claim 25, wherein the gRNAs comprise one or more of SEQ ID NOs: 402-407 or a sequence with at least 80%, 82%, 85%, 87%, 90%, 92%, 94%, 95%, 97%, 98% or 99% nucleic acid sequence identity thereto.

44. The method of claim 25, wherein the gRNAs comprise one or more of SEQ ID NOs: 408-413 or a sequence with at least 80%, 82%, 85%, 87%, 90%, 92%, 94%, 95%, 97%, 98% or 99% nucleic acid sequence identity thereto.

45. A population of cells obtained by the method of claim 25.

46. An isolated human cell selected by the method of claim 25.

47. An isolated nucleic acid comprising one of SEQ ID NOs: 402-424 or a combination thereof.

48. A vector comprising at least one of SEQ ID NOs: 402-424.

49. A complex comprising a Cas polypeptide and one of SEQ ID NOs: 402-424.


SEQUENCE LISTING (451 sequences)

SEQ ID NO: 1 - RNA, 20 nt, synthetic construct
  source 1..20: mol_type = other RNA
atttcttcccctcaccacag

SEQ ID NO: 2 - RNA, 20 nt, synthetic construct
  source 1..20: mol_type = other RNA
atcctcacgatgaaccccag

SEQ ID NO: 3 - RNA, 20 nt, synthetic construct
  source 1..20: mol_type = other RNA
gttgaatagagtgcctgctg

SEQ ID NO: 4 - RNA, 20 nt, synthetic construct
  source 1..20: mol_type = other RNA
caggtgtttaacagatgtca

SEQ ID NO: 5 - RNA, 20 nt, synthetic construct
  source 1..20: mol_type = other RNA
tatagtccagtatagagggg

SEQ ID NO: 6 - RNA, 20 nt, synthetic construct
  source 1..20: mol_type = other RNA
tgaatgctgagtgtacaatg

SEQ ID NO: 7 - RNA, 3780 nt, Homo sapiens
  source 1..3780: mol_type = genomic RNA
ggatgggatctcctgctcctgttcctcccgctcctgcccacggcgggctgcgcgggacgtagcgcgctgggtggtcgcaaaaggaagcccagccgtcgtgtgaggcccgcacgtgcaccgccttctcgaaggagcggttcagcacgcgtaccaacccgcgcagcaccggcgggcggcccccaggcacccacaccccggcacccccggggaccgctccgggaggcggcagcagcgcctccagctccaccatgacgcgccccaagcgctccagacggcccggcgcgggcggcagcgaaaatgtggggaccagccaggccatcaggcccattctgggcttttccagaagggtctgaagccagtcttgtagagggctggagtggttgttggacgactagaaccctgggctttgcagggtgctgggagctggaagtcttcaccatggcctgtcaggagaagcccctgggggctggtctaccatgctgaccagtggttgaatagagtgcctgctgaggtcaagagatgcagccgccaattcctggacaacctaaaagcctcacaagcttcaagtctccaaggactctccaaatcccaggatctccctgccatcacatcctagaatttaaaacctgagaacatcaagttcaggaaagcttggggatttcaaagcccagcaccaaagaaagcgggacccacactcgccagctctgtgatcttgggcaagttgcttgactacttatacctccgtttccctcatctgtataacaggaatacaataatgatgagatggagctcaaggggccatggtgaggattgaacatgatcacttgtgagacatgttcagcactgtatctgacccatggaaaattcaagataaacatcagctactgagatgatggcggatatttggaatcctaaatccttggaaactggggctttttgaagtaaaagaccccaaaggctgagggcctcagaagcatcaggccatgatgttcctgaaacaagagggtcagggtcccaatgggcctctggggttcatcgtgaggatggatgcattaatattggggacctgctagggaccttcccagtgggacagtggctgggtcagggcactcaagccctaaaacgtgatgaggcgagacttttctctctttcctcattcagtaactgtcagtagattctgggagccagggattctccgactcttcaagtccatgaattttaggggatgacagtgggctctccgctttctcctccatgaagtaacttacatgcccctcaccctctgtgggaggggtgttgcagggggtgcagaactcccctcgccgggtagttcaagcaatggggaccatatcaattccatctatagggaaactgaggcctggagtagggcgaggcctctgggaacccagccctattctgtctctttccctggcatttcccatccacacatagagcttcagattctctttctttccccagagaccctcaaatatcctctcactcacagaatggtgtctctgcctgcctcgggttggccctgtgatttattttagttcttttcccttgttttttttttttcaaactctatacacttttgttttaaaaactgtggtttctcatgagccctattatctcattgatacctctcacctctgtggtgaggggaagaaatcatattttcagatgactcgtaaagggcaaagaaaaaaacccaaaatttcaaaatttccgtttaagtctcataatcaagaaaaggagaaacacagagagagagaaaaaaaaaactatgagaacccctccccaccccgtgattatcagcgcacacactcatcgaaaaaaatttggattattagaagagagaggtctgcggcttccacaccgtacagcgtggtttttcttctcggtataaaagcaaagttgtttttgatacgtgacagtttcccacaagccaggctgatccttttctgtcagtccacttcaccaaggtgagtgtccctgctctcccctaccagatgtgggccccattggaggagatggcagggaggtaggcacggcgggggggtcaggggccctctggtacagtgggatgtacccagctaccgtgattccagccaggtaaggtctttaaaaaaataatagaataaatggcagaagacttaaaggtgaagtacttaatgcatgggcaggggcaaaaataaatgaattcatcaattgatacgtagatggataggatttttaaaatatgtcagctgccagtctttatggatttcagagctttgaacaacttgaaatgctgtccaggtgcaactgaaaatttcaactgcatctctctcatctctttgtctcttagctcttgatgcctctcatccttctctgtctctctgctcaccctgtcttgctcttttacatgtttctctgcctctctttctcccgccatctgtctgtcgctacactcagactctctttccccgcatctctcctggcctctgaccatctcccctatctctgtctcctccatcttgaccacactccatcgatctcctttcctccatctctcccgctctcttttcttgccttcccccacctgccttcccagccagcatccagcctagagctttgtacacagctggcgtttaataatagtaatgtcgccgaggacctttacattttatgagagtgagcatcagttctgttcaccctagtagcaggaggattgcaaggtgccggggcaagctgagcacttaactgaactttgcttgtatcgctttagaggagacccttggggcgggggggcagggagcacttcacaccattgtcaaccgtagtttagctgtgtggacctaccccaggactgcatgtgtgtgaccagagtccatattcaggaagcaaacaggttgttcagggctggggtgagctcgattctgcaggcttagcacagcgtttggcacaaagtaagtgctcacgaagtgttagctattagtatttctgtaaccaaattagaatcatgctatatattggttttgttcctgctttattcaattaacagtagtgtttttgaagcttactaaaaattgtttgaacacatgatctttcatgatggacaagactgtcctcacatcagcatgaactttgattgatttgaccaagtgccgattgttggacattccggttgtttctcgtttttttctataaacatccctgactgcttccttggattcctgaaaagggagttgctggggcaaagggggcgtgcatttttaaagtttatgaccctctctgctaattctccttccaaaaggttgtcctattttatgctccctctacttaagctaaccctaggtgagtattattatgttttaattatttcccaacatgacaggtaagaaaaacacattatcacattgttgaaatttgcatcttgtgattacttttactattcattggccattcatgcttttttttttttttttttggttgtctggtcatgtccttactcctaatagggtgttcatcttatttttcccttattgatttgccacagctctttatttggaatggttattaacctttcatcagccattacatatatagcaaatattttccccaattcatcatttgctttttttctatttgtttatggtgttttttgtgatgcttatgatggttttatacagtcaaataggttagtctttttttctgtggcttctgtctctggttttgtgcttagaaagtcctttcctacttgaaaatgagataaatgttcacctatgttggcttcta

SEQ ID NO: 8 - RNA, 310 nt, Homo sapiens
  source 1..310: mol_type = genomic RNA
ggatgggatctcctgctcctgttcctcccgctcctgcccacggcgggctgcgcgggacgtagcgcgctgggtggtcgcaaaaggaagcccagccgtcgtgtgaggcccgcacgtgcaccgccttctcgaaggagcggttcagcacgcgtaccaacccgcgcagcaccggcgggcggcccccaggcacccacaccccggcacccccggggaccgctccgggaggcggcagcagcgcctccagctccaccatgacgcgccccaagcgctccagacggcccggcgcgggcggcagcgaaaatgtggggaccag

SEQ ID NO: 9 - RNA, 310 nt, Homo sapiens; identical to SEQ ID NO: 8
  source 1..310: mol_type = genomic RNA

SEQ ID NO: 10 - RNA, 3433 nt, Homo sapiens
  source 1..3433: mol_type = genomic RNA
gtctgaagccagtcttgtagagggctggagtggttgttggacgactagaaccctgggctttgcagggtgctgggagctggaagtcttcaccatggcctgtcaggagaagcccctgggggctggtctaccatgctgaccagtggttgaatagagtgcctgctgaggtcaagagatgcagccgccaattcctggacaacctaaaagcctcacaagcttcaagtctccaaggactctccaaatcccaggatctccctgccatcacatcctagaatttaaaacctgagaacatcaagttcaggaaagcttggggatttcaaagcccagcaccaaagaaagcgggacccacactcgccagctctgtgatcttgggcaagttgcttgactacttatacctccgtttccctcatctgtataacaggaatacaataatgatgagatggagctcaaggggccatggtgaggattgaacatgatcacttgtgagacatgttcagcactgtatctgacccatggaaaattcaagataaacatcagctactgagatgatggcggatatttggaatcctaaatccttggaaactggggctttttgaagtaaaagaccccaaaggctgagggcctcagaagcatcaggccatgatgttcctgaaacaagagggtcagggtcccaatgggcctctggggttcatcgtgaggatggatgcattaatattggggacctgctagggaccttcccagtgggacagtggctgggtcagggcactcaagccctaaaacgtgatgaggcgagacttttctctctttcctcattcagtaactgtcagtagattctgggagccagggattctccgactcttcaagtccatgaattttaggggatgacagtgggctctccgctttctcctccatgaagtaacttacatgcccctcaccctctgtgggaggggtgttgcagggggtgcagaactcccctcgccgggtagttcaagcaatggggaccatatcaattccatctatagggaaactgaggcctggagtagggcgaggcctctgggaacccagccctattctgtctctttccctggcatttcccatccacacatagagcttcagattctctttctttccccagagaccctcaaatatcctctcactcacagaatggtgtctctgcctgcctcgggttggccctgtgatttattttagttcttttcccttgttttttttttttcaaactctatacacttttgttttaaaaactgtggtttctcatgagccctattatctcattgatacctctcacctctgtggtgaggggaagaaatcatattttcagatgactcgtaaagggcaaagaaaaaaacccaaaatttcaaaatttccgtttaagtctcataatcaagaaaaggagaaacacagagagagagaaaaaaaaaactatgagaacccctccccaccccgtgattatcagcgcacacactcatcgaaaaaaatttggattattagaagagagaggtctgcggcttccacaccgtacagcgtggtttttcttctcggtataaaagcaaagttgtttttgatacgtgacagtttcccacaagccaggctgatccttttctgtcagtccacttcaccaaggtgagtgtccctgctctcccctaccagatgtgggccccattggaggagatggcagggaggtaggcacggcgggggggtcaggggccctctggtacagtgggatgtacccagctaccgtgattccagccaggtaaggtctttaaaaaaataatagaataaatggcagaagacttaaaggtgaagtacttaatgcatgggcaggggcaaaaataaatgaattcatcaattgatacgtagatggataggatttttaaaatatgtcagctgccagtctttatggatttcagagctttgaacaacttgaaatgctgtccaggtgcaactgaaaatttcaactgcatctctctcatctctttgtctcttagctcttgatgcctctcatccttctctgtctctctgctcaccctgtcttgctcttttacatgtttctctgcctctctttctcccgccatctgtctgtcgctacactcagactctctttccccgcatctctcctggcctctgaccatctcccctatctctgtctcctccatcttgaccacactccatcgatctcctttcctccatctctcccgctctcttttcttgccttcccccacctgccttcccagccagcatccagcctagagctttgtacacagctggcgtttaataatagtaatgtcgccgaggacctttacattttatgagagtgagcatcagttctgttcaccctagtagcaggaggattgcaaggtgccggggcaagctgagcacttaactgaactttgcttgtatcgctttagaggagacccttggggcgggggggcagggagcacttcacaccattgtcaaccgtagtttagctgtgtggacctaccccaggactgcatgtgtgtgaccagagtccatattcaggaagcaaacaggttgttcagggctggggtgagctcgattctgcaggcttagcacagcgtttggcacaaagtaagtgctcacgaagtgttagctattagtatttctgtaaccaaattagaatcatgctatatattggttttgttcctgctttattcaattaacagtagtgtttttgaagcttactaaaaattgtttgaacacatgatctttcatgatggacaagactgtcctcacatcagcatgaactttgattgatttgaccaagtgccgattgttggacattccggttgtttctcgtttttttctataaacatccctgactgcttccttggattcctgaaaagggagttgctggggcaaagggggcgtgcatttttaaagtttatgaccctctctgctaattctccttccaaaaggttgtcctattttatgctccctctacttaagctaaccctaggtgagtattattatgttttaattatttcccaacatgacaggtaagaaaaacacattatcacattgttgaaatttgcatcttgtgattacttttactattcattggccattcatgcttttttttttttttttttggttgtctggtcatgtccttactcctaatagggtgttcatcttatttttcccttattgatttgccacagctctttatttggaatggttattaacctttcatcagccattacatatatagcaaatattttccccaattcatcatttgctttttttctatttgtttatggtgttttttgtgatgcttatgatggttttatacagtcaaataggttagtctttttttctgtggcttctgtctctggttttgtgcttagaaagtcctttcctacttgaaaatgagataaatgttcacctatgttggcttcta

SEQ ID NO: 11 - RNA, 3727 nt, Homo sapiens
  source 1..3727: mol_type = genomic RNA
gtaaaaccccccgccagcggggacggggcacagcggtgagggctcggggcaagcctcctcttcctcctccccttcatccccatcctcgccatcgtcgtcctcgtcggccccgccgccgccgccgccatcttgcccggccgccgccgccatcttgcccgccccgggcccgccccacggccggtagcggcgcaactgcgccagcggcagccccaaggcctcgtcggcgaacagcaccctccgcggggccaccgccgcctcgaccgaggtgcggggctcacccgcggccggcgaggggggcgcggaatgccgcagcgggggctccacaggggccgtacgcgccatatcggcggcggcggcggcaccgacggcaccggcggcgggaccggcgggggcagggcctgaaggggcgggcagccaatgggaaggccagcgcggaggggtacggctcacgggggcataggcggtgagatgaagacaagtattgcagagggccaatgggcagccatgcacggatgacgtacagcgaattgagagccggagaagcacgcagccaatggggatgcccgggagtgggcggtgtagggggcgggatgatcgaggaatcaatggagaggccggaatgggggtggtgcgaaatccatgatgaagaaaacagaagcagacagctaatgcggaagtctgagcgagagaggcatgtgggcggtgggaaaagtggaatggctacaggcgatgagggcagccgggaacgaacgcagaggcccaaaaagtgggagatcccgcgggttgcgtaggcggtggaacggtagctagagagagagaacagccaatggagagccacgcgtgggatgacgacaatggctgcagcgagccaatggggaggcttgtgagcgtgcactgcggcttcgaaggggtggtgccaaggtggagctagccaatacgaaggctaaaaggagaccaagaggcgggtgtgtagggagccgagccaatggaggtggaggctgaaggcgggagcgaggacgagagctggcggatagccaatgagggagccagggtgaaggcgtagagacctcaaaggacagtactagtatgggatgagccaatgccgaggctagatagtgcagcgggacggagggaggacttggaggcaagatggggaaaaaagcagagccgacgggggcagtgtaggtggtgtgaagggaggcatgagtagccaataaggaggctggagaggaggacgctgtgtagacagagtctgaggtattgggtggccggacagcagaaccatagaacgcaggcagccattaggaaaaatgtgcttctcagggatggggttaccctctccctaaccctccgtgactccccattgccccaggaatacccagagcccctgaaggcctagaccacaccttggtgtgtgaactaggaaagtgaaaatgactgtcctgttgcagttagttacttactgggtcctccctgcccaactacaaccttttcttgagcagtgccacaagccaagaatgcccctgattgttagccgcaagcactctccaggacttgatactattgttgaatgaatggccgaataggaaatttaactttctgaaggattcacttttttaaatttcagctcagcaaacactgacctgactgtgtgctaccagaagttgattgggcatatgggtagcttctgactaatggaatcacaatcaaagccctgtccttcttgcccaacctgacactccacaccccacaggggcctcacctcccttctttctgtccccccatctctgcttctactgttccctctcccaagagtgctgttccttctgctgcttccatctagtaaactcctttcgctctccctttggggtctcaccttctgtgggaactcaactgcagcttttgtccgctgaacacatccattcatacattccttctccatcctgggaagatttactgaacacctactgagtgccaggttctgagcatgctgttatctcattagaacctcacagcatcctaggagggaggtgatccttccagcagcccctctccccaggtctccatgttcaggatgtccatgagttagaaatgaatatgttcagctgcaagtaacagaaaaatcataaaataatgggggcttaagcaagacagaactttatttttttctcacctaaaagttcaagctggcaaatagtccagggactccttcctgcccacctggtttgggcattgtctgtcccgatctggctccaagactctcagtcttcaacaaatccttttggctgtctattaaaaatatttaccaaatccatcctcttctcaccacctccactgccagcactatcaccagtcacctagactactgcagtagccagctccttgctggtcccctgcttccacattcaggccctagaccccattgtacactcagcattcaggggcttcctgttaaatcctaacacggtttgtgtttctcctctgcttgcaaccctgtcatgccttcctcctcactcagcgttgaagccaaagtccttaccatggcctacaaggtcctgcgtggtctaacctccgagtcccctttctgacctcacctccagcttctcttcttgctctctccacactaaccacaccagcccccttgtagaccttgaacaaatcaggtacattcccacctcaggacctttgccctggctgtttcctttggagttcctcagatatctagctggctccctccctcatctccttcaagtctttgtcagaggcccctgtctctggggacttttcctgacccgtttaatcttgcctactcacccccaactcccagccctcccagtctccttcccgagaagccctccatgagagaggatatagtccagtatagaggggaggtagcgacctctacccctctctgccctcctgaccccagcccccattctgcaaggatcttggctttcagcagccgtagcatttccctctcacaccctgggcctgcccaaggggccagctcacattctcggccaccatcctcacttcgtttgcctcctatatctctggccactctctttcatggagtgagtgacagtaagggacagatggtctggggaaggtataaggagaatgcagcccacacctctgatcctgagagagaaaactagcccccttttccaggagggggacaggtgctggggcagggaacaatgcctctgctgatagaactggcctggtttataattacgcaggaggtgtagggaacattccgacccaagtctgcagggagcaaggggttattaactatgggaacatcagtgcaagggatcattaaccttaggaccatcactgcaaggggtcattaaccttggggccatcactagaatcagctccccaggactccttcctgacatgcatgctcctcaccccctcccttgctccctcccacatgccgtgacatctgttaaacacctgctgttcacagggtccctgcaggcctccaagcaggggacctcatcccaccttggtgcctcccctggctgtccaacacacccaagccatttttgggtctcagatgtgtcccaccaactcagaagcctcaaccagcgaaagttttgttttgaactaatggtggggagggttgggtgtttttttttttttttaattgttgttggctgatttttttaaggtctttttttttctattctttttttttttttttcttgtcttcacaaag

SEQ ID NO: 12 - RNA, 149 nt, Homo sapiens
  source 1..149: mol_type = genomic RNA
gtgagtctgagttgaggaaagaggccctcctttttacgagggcttagaaggttgggggaggaaagggggcaaaggggtgaaggggtggatcttggccaccagatttgtaccatgtaaataaaaagccctttccaatttctcttccccag

SEQ ID NO: 13 - DNA, 7656 bp, Homo sapiens
  source 1..7656: mol_type = genomic DNA
ggatgggatctcctgctcctgttcctcccgctcctgcccacggcgggctgcgcgggacgtagcgcgctgggtggtcgcaaaaggaagcccagccgtcgtgtgaggcccgcacgtgcaccgccttctcgaaggagcggttcagcacgcgtaccaacccgcgcagcaccggcgggcggcccccaggcacccacaccccggcacccccggggaccgctccgggaggcggcagcagcgcctccagctccaccatgacgcgccccaagcgctccagacggcccggcgcgggcggcagcgaaaatgtggggaccaggtaaaaccccccgccagcggggacggggcacagcggtgagggctcggggcaagcctcctcttcctcctccccttcatccccatcctcgccatcgtcgtcctcgtcggccccgccgccgccgccgccatcttgcccggccgccgccgccatcttgcccgccccgggcccgccccacggccggtagcggcgcaactgcgccagcggcagccccaaggcctcgtcggcgaacagcaccctccgcggggccaccgccgcctcgaccgaggtgcggggctcacccgcggccggcgaggggggcgcggaatgccgcagcgggggctccacaggggccgtacgcgccatatcggcggcggcggcggcaccgacggcaccggcggcgggaccggcgggggcagggcctgaaggggcgggcagccaatgggaaggccagcgcggaggggtacggctcacgggggcataggcggtgagatgaagacaagtattgcagagggccaatgggcagccatgcacggatgacgtacagcgaattgagagccggagaagcacgcagccaatggggatgcccgggagtgggcggtgtagggggcgggatgatcgaggaatcaatggagaggccggaatgggggtggtgcgaaatccatgatgaagaaaacagaagcagacagctaatgcggaagtctgagcgagagaggcatgtgggcggtgggaaaagtggaatggctacaggcgatgagggcagccgggaacgaacgcagaggcccaaaaagtgggagatcccgcgggttgcgtaggcggtggaacggtagctagagagagagaacagccaatggagagccacgcgtgggatgacgacaatggctgcagcgagccaatggggaggcttgtgagcgtgcactgcggcttcgaaggggtggtgccaaggtggagctagccaatacgaaggctaaaaggagaccaagaggcgggtgtgtagggagccgagccaatggaggtggaggctgaaggcgggagcgaggacgagagctggcggatagccaatgagggagccagggtgaaggcgtagagacctcaaaggacagtactagtatgggatgagccaatgccgaggctagatagtgcagcgggacggagggaggacttggaggcaagatggggaaaaaagcagagccgacgggggcagtgtaggtggtgtgaagggaggcatgagtagccaataaggaggctggagaggaggacgctgtgtagacagagtctgaggtattgggtggccggacagcagaaccatagaacgcaggcagccattaggaaaaatgtgcttctcagggatggggttaccctctccctaaccctccgtgactccccattgccccaggaatacccagagcccctgaaggcctagaccacaccttggtgtgtgaactaggaaagtgaaaatgactgtcctgttgcagttagttacttactgggtcctccctgcccaactacaaccttttcttgagcagtgccacaagccaagaatgcccctgattgttagccgcaagcactctccaggacttgatactattgttgaatgaatggccgaataggaaatttaactttctgaaggattcacttttttaaatttcagctcagcaaacactgacctgactgtgtgctaccagaagttgattgggcatatgggtagcttctgactaatggaatcacaatcaaagccctgtccttcttgcccaacctgacactccacaccccacaggggcctcacctcccttctttctgtccccccatctctgcttctactgttccctctcccaagagtgctgttccttctgctgcttccatctagtaaactcctttcgctctccctttggggtctcaccttctgtgggaactcaactgcagcttttgtccgctgaacacatccattcatacattccttctccatcctgggaagatttactgaacacctactgagtgccaggttctgagcatgctgttatctcattagaacctcacagcatcctaggagggaggtgatccttccagcagcccctctccccaggtctccatgttcaggatgtccatgagttagaaatgaatatgttcagctgcaagtaacagaaaaatcataaaataatgggggcttaagcaagacagaactttatttttttctcacctaaaagttcaagctggcaaatagtccagggactccttcctgcccacctggtttgggcattgtctgtcccgatctggctccaagactctcagtcttcaacaaatccttttggctgtctattaaaaatatttaccaaatccatcctcttctcaccacctccactgccagcactatcaccagtcacctagactactgcagtagccagctccttgctggtcccctgcttccacattcaggccctagaccccattgtacactcagcattcaggggcttcctgttaaatcctaacacggtttgtgtttctcctctgcttgcaaccctgtcatgccttcctcctcactcagcgttgaagccaaagtccttaccatggcctacaaggtcctgcgtggtctaacctccgagtcccctttctgacctcacctccagcttctcttcttgctctctccacactaaccacaccagcccccttgtagaccttgaacaaatcaggtacattcccacctcaggacctttgccctggctgtttcctttggagttcctcagatatctagctggctccctccctcatctccttcaagtctttgtcagaggcccctgtctctggggacttttcctgacccgtttaatcttgcctactcacccccaactcccagccctcccagtctccttcccgagaagccctccatgagagaggatatagtccagtatagaggggaggtagcgacctctacccctctctgccctcctgaccccagcccccattctgcaaggatcttggctttcagcagccgtagcatttccctctcacaccctgggcctgcccaaggggccagctcacattctcggccaccatcctcacttcgtttgcctcctatatctctggccactctctttcatggagtgagtgacagtaagggacagatggtctggggaaggtataaggagaatgcagcccacacctctgatcctgagagagaaaactagcccccttttccaggagggggacaggtgctggggcagggaacaatgcctctgctgatagaactggcctggtttataattacgcaggaggtgtagggaacattccgacccaagtctgcagggagcaaggggttattaactatgggaacatcagtgcaagggatcattaaccttaggaccatcactgcaaggggtcattaaccttggggccatcactagaatcagctccccaggactccttcctgacatgcatgctcctcaccccctcccttgctccctcccacatgccgtgacatctgttaaacacctgctgttcacagggtccctgcaggcctccaagcaggggacctcatcccaccttggtgcctcccctggctgtccaacacacccaagccatttttgggtctcagatgtgtcccaccaactcagaagcctcaaccagcgaaagttttgttttgaactaatggtggggagggttgggtgtttttttttttttttaattgttgttggctgatttttttaaggtctttttttttctattctttttttttttttttcttgtcttcacaaagccaggccatcaggcccattctgggcttttccagaagggtgagtctgagttgaggaaagaggccctcctttttacgagggcttagaaggttgggggaggaaagggggcaaaggggtgaaggggtggatcttggccaccagatttgtaccatgtaaataaaaagccctttccaatttctcttccccaggtctgaagccagtcttgtagagggctggagtggttgttggacgactagaaccctgggctttgcagggtgctgggagctggaagtcttcaccatggcctgtcaggagaagcccctgggggctggtctaccatgctgaccagtggttgaatagagtgcctgctgaggtcaagagatgcagccgccaattcctggacaacctaaaagcctcacaagcttcaagtctccaaggactctccaaatcccaggatctccctgccatcacatcctagaatttaaaacctgagaacatcaagttcaggaaagcttggggatttcaaagcccagcaccaaagaaagcgggacccacactcgccagctctgtgatcttgggcaagttgcttgactacttatacctccgtttccctcatctgtataacaggaatacaataatgatgagatggagctcaaggggccatggtgaggattgaacatgatcacttgtgagacatgttcagcactgtatctgacccatggaaaattcaagataaacatcagctactgagatgatggcggatatttggaatcctaaatccttggaaactggggctttttgaagtaaaagaccccaaaggctgagggcctcagaagcatcaggccatgatgttcctgaaacaagagggtcagggtcccaatgggcctctggggttcatcgtgaggatggatgcattaatattggggacctgctagggaccttcccagtgggacagtggctgggtcagggcactcaagccctaaaacgtgatgaggcgagacttttctctctttcctcattcagtaactgtcagtagattctgggagccagggattctccgactcttcaagtccatgaattttaggggatgacagtgggctctccgctttctcctccatgaagtaacttacatgcccctcaccctctgtgggaggggtgttgcagggggtgcagaactcccctcgccgggtagttcaagcaatggggaccatatcaattccatctatagggaaactgaggcctggagtagggcgaggcctctgggaacccagccctattctgtctctttccctggcatttcccatccacacatagagcttcagattctctttctttccccagagaccctcaaatatcctctcactcacagaatggtgtctctgcctgcctcgggttggccctgtgatttattttagttcttttcccttgttttttttttttcaaactctatacacttttgttttaaaaactgtggtttctcatgagccctattatctcattgatacctctcacctctgtggtgaggggaagaaatcatattttcagatgactcgtaaagggcaaagaaaaaaacccaaaatttcaaaatttccgtttaagtctcataatcaagaaaaggagaaacacagagagagagaaaaaaaaaactatgagaacccctccccaccccgtgattatcagcgcacacactcatcgaaaaaaatttggattattagaagagagaggtctgcggcttccacaccgtacagcgtggtttttcttctcggtataaaagcaaagttgtttttgatacgtgacagtttcccacaagccaggctgatccttttctgtcagtccacttcaccaaggtgagtgtccctgctctcccctaccagatgtgggccccattggaggagatggcagggaggtaggcacggcgggggggtcaggggccctctggtacagtgggatgtacccagctaccgtgattccagccaggtaaggtctttaaaaaaataatagaataaatggcagaagacttaaaggtgaagtacttaatgcatgggcaggggcaaaaataaatgaattcatcaattgatacgtagatggataggatttttaaaatatgtcagctgccagtctttatggatttcagagctttgaacaacttgaaatgctgtccaggtgcaactgaaaatttcaactgcatctctctcatctctttgtctcttagctcttgatgcctctcatccttctctgtctctctgctcaccctgtcttgctcttttacatgtttctctgcctctctttctcccgccatctgtctgtcgctacactcagactctctttccccgcatctctcctggcctctgaccatctcccctatctctgtctcctccatcttgaccacactccatcgatctcctttcctccatctctcccgctctcttttcttgccttcccccacctgccttcccagccagcatccagcctagagctttgtacacagctggcgtttaataatagtaatgtcgccgaggacctttacattttatgagagtgagcatcagttctgttcaccctagtagcaggaggattgcaaggtgccggggcaagctgagcacttaactgaactttgcttgtatcgctttagaggagacccttggggcgggggggcagggagcacttcacaccattgtcaaccgtagtttagctgtgtggacctaccccaggactgcatgtgtgtgaccagagtccatattcaggaagcaaacaggttgttcagggctggggtgagctcgattctgcaggcttagcacagcgtttggcacaaagtaagtgctcacgaagtgttagctattagtatttctgtaaccaaattagaatcatgctatatattggttttgttcctgctttattcaattaacagtagtgtttttgaagcttactaaaaattgtttgaacacatgatctttcatgatggacaagactgtcctcacatcagcatgaactttgattgatttgaccaagtgccgattgttggacattccggttgtttctcgtttttttctataaacatccctgactgcttccttggattcctgaaaagggagttgctggggcaaagggggcgtgcatttttaaagtttatgaccctctctgctaattctccttccaaaaggttgtcctattttatgctccctctacttaagctaaccctaggtgagtattattatgttttaattatttcccaacatgacaggtaagaaaaacacattatcacattgttgaaatttgcatcttgtgattacttttactattcattggccattcatgcttttttttttttttttttggttgtctggtcatgtccttactcctaatagggtgttcatcttatttttcccttattgatttgccacagctctttatttggaatggttattaacctttcatcagccattacatatatagcaaatattttccccaattcatcatttgctttttttctatttgtttatggtgttttttgtgatgcttatgatggttttatacagtcaaataggttagtctttttttctgtggcttctgtctctggttttgtgcttagaaagtcctttcctacttgaaaatgagataaatgttcacctatgttggcttcta

SEQ ID NO: 14 - DNA, 7656 bp, Homo sapiens; identical to SEQ ID NO: 13
  source 1..7656: mol_type = genomic DNA

SEQ ID NO: 15 - DNA, 78 bp, Mus musculus
  source 1..78: mol_type = genomic DNA
ccagtcttgttatttccaaaaaggtgagttaagatgaggaaagtcagtctcttttttgttgttgttgttggggcgggg

SEQ ID NO: 16 - DNA, 68 bp, Mus musculus
  source 1..68: mol_type = genomic DNA
tcatgtgaataaaaacgtatttctactttctcttcctcaggcctgaagccagtcttgcaaagaggtgg

SEQ ID NO: 17 - DNA, 64 bp, Homo sapiens
  source 1..64: mol_type = genomic DNA
ccattctgggcttttccagaagggtgagtctgagttgaggaaagaggccctcctttttacgagg

SEQ ID NO: 18 - DNA, 68 bp, Homo sapiens
  source 1..68: mol_type = genomic DNA
ccatgtaaataaaaagccctttccaatttctcttccccaggtctgaagccagtcttgtagagggctgg

SEQ ID NO: 19 - skipped

SEQ ID NO: 20 - DNA, 2301 bp, Homo sapiens
  source 1..2301: mol_type = genomic DNA
ttcaaatatttctttgatctgctgaacaactcactgatgtaggtattattatcccctccttagagatgaggtaactgaggcacagagaggttaagtaacttgccccaggtcacacagctgataggtggcagagctggaattgttcaaaattgcatatcctaaccctttattgagttgtgaaatcaatttactgggtttcaacaagcattaaaaagaaaaaggaaatagcataagaaatgccagtgtatcaccacatgcagtaaaggtaagtattgtttcactcgaagaacaaccatttttcagttatttatatgtctgtttacacgtgtgggtgtgctgggttatgatgtagaatggatttcttactatgggtcgtggccaaagaatgaagtcatggctgttgaagaagctcatggcacgagaaactgctctccctctaccccactggatcacccgcaagtcccagagttgaggctgacacacttgttggggaaggcaaagcagtgccccacataacttggtggctttcacagccaccttcaaaccctgttcttttcagaaagcagatggctcagggtaactgcaattctgagtatcgtggggcaggtttctgaagccactcatccccccggaaagtcagcgttatcttcaggttgactacatgggagccggggtctgctgagtttccctttgggtttcagtgcctgacccactttgggctggcaaaattctttgaaaacatgaatgctgggggtgctccagaggactcgggtggtggtggcaatggcagtctgtgactgctctgaaagtctgctttgcttttctccacagggcttgtcagccctcacccgctcgcttactctgtgactggcgaatcacctttcttggctttcttggctggcctaggccggggccaacaccacctctttccaaatcctcaccctctggtctcctcggggcctatcagcttggcagccattgtgtttcctgatggccggaggaatttgcacgcccaggagactggcgtgcaggcctgagatggccccttttagtcgacaccagcttgactagtgctcactagcacccaaatgatgcatgtccaagattttccagatctgtgtgccctggcccctatggctcactgcccttgaggggatgccacgtggtacttgtggggctggtgccaaaagaacaggtttccttcttgaaaacgagcaggcatactgcaggtacagttttgttcttaatcttctccctccccatttttctaagaacccctcttctctgttaccgatcagtgagtcagtatacatttgtacttgatttctcttactatcctcatgttgattgaagtcatagctgcccttgagtttttactgtgaaagacggttcaaagataacttgtttctttttaagcccacaatttcaaactctcttcaaagtggagccctcctggagtgtttgttaccagcgtggttgtgtagtcagtgagtgtagagatgcagttccttgagttttagtttttgcatttgtaaaaaggaagggtgttgttttgaaggatagatgtgaaggttttcaaatgccttggtgtgtcaatgagaggggcccatggtggaggaggtgaacaatacatgcttgtgctttctgctttcatatctgactttggagaacgacttgtttgcttctgtcgatgttgtggatcttgggattggctcaatggcgtgacctgctttttggatgttctcgccctcctcagccatggaaagggtgctctgggggctgaaggattgattgtgtatttgtttttctttctccttcctccaactgaattgtggagtcctttacctgctggctagctgattcctgagtgttctcctttttctgtctcacatctatgactgcagtggcttttagaagcctgtttgtaatatatgtccggactaggccagatggaggagaaggcttgcctgctactgcgcacaggtgggagggctggctttctgtctgtctgtgggccttcttgaagaggcttggtttagaagatcctaggaggaggatgttttctgtcatgaaggactatggtaacaaaaagaagtaagttagtgcagcctggcagaaattgtgttgaaacaaaagtccaaagacctggattttaggaccacggaggggattggtgtgagaccaagctggtttgctctgaatctctctttctcatctgtgatgtgtgggaggtggcagctgggctcccagagtcacccaccctaggccctgtagtattctgattcaagtacctctggtggg

SEQ ID NO: 21 - DNA, 1201 bp, Homo sapiens
  source 1..1201: mol_type = genomic DNA
ggtgctatgattatccccatttagtagaggggagacaggtagctcagagagatgagccagcttgctcaaagcctctcagctagtgagtgacagggccagggaagaaatgctagggacatttatttttttctattttttttttttaaactggtcctctagaaagcgtggcatgattcagggcaaattctggatttcactcttggtgttcccagcatgtcgtgctttccttttacctttttttttttttttgctttatcgaggtatacctacataaagtgtactgatattctgtgtatagcccagtggatttgtacatatgtatgcacttgtgtagccaccacccagatgaagatactgagcaggtccagtaccccagagctaccttcatttctatcactttcccaagctgtcatccccgccggctgtcatgggaaccctgtctgtaagatgcgacagtttgggtaaaggagtttggtcattttaaagagtgtgaaaggcagagaacagagaaatcaaaaccttgcagggccaaggtgggtggagagggtgtttttcttttaacatacatgggcggttttaaggagaaattgaagcagcctgttcagacaattgttttggtatctggccccaggtctgtggttcctaacatgacttgtgatattattttaagtgggcagatggctttttgatagcttctttatctttcgatctcagctcttgcaaaggggaggttggtgctcattgcaagatcagcgataagggtttctttgtaggtcggtggctttcttggtgagtacatttcaacatattattgttttagaacctgtgtgctgccagtgacttgcagcactgttgaagactagccaccctttgtgacctagccctcttgggaaatggcggaggatctcagggtatatcccttacctgtgggagccctatcagagggcttcctgttgaggaaatgttggctgtaggccctctgtgcactgagcacagccacatcaggtgagggcatgggagaagtccgtggtagccatcaggatagagttcagaaacccaaatggctgctttccctgggttgctggaccaggcatttggtaactctaaagcttagagatgattcatcgacaagcatttattgactgcctactgtgtgctgggcacagtgctaggttccagcagggaaagagatgcagagtgg

SEQ ID NO: 22 - DNA, 621 bp, Homo sapiens
  source 1..621: mol_type = genomic DNA
cctgtggtcacttctgaagctgcctggacacttggccagagctaagaattctccccaaacacatgtgggatggcctgactcagcaaagcatagatacattctcagacagggacatggagatgatctgtctgggggtagaggacctagagggccgggctgggcagccggcttcctgcactgtctgttgggacgtccctttctgactgggtttctcagaagctgaatgggggatgtttctgggacacagattatgttttcatatcggggtctgcatctgggccctgttgtcacagcccccgacttgcccagatttttccgccattgacgtcatggcggccggatgcgccgggcttcatcgacaccacggaggaagagaagagggcagataccccaccccacaggtttcgttccgagaactggctgccctgtcctgcagcaggcttggcccaggtggggtgacatgggtgctggtggatgtggtaggtgatgtccatctggccactatgacaagcccctagctctgaagacctggcccttcttgggttgtggagaggacccaggtttgaagctctgagagtgccaggcaggctccacagatactgggacccctggggtcttcaa

SEQ ID NO: 23 - DNA, 211 bp, Homo sapiens
  source 1..211: mol_type = genomic DNA
gcctcctcctctcctgagacagggattgggaggtcggggagagcctccaatctctgaggcctggcaggtggggattttcttggccctgcaacatctgcataagtcacagacttgcctgggacccagaaaccacttcctgtgccccagccagcccccctcccgcccagtgccacagtaaaggtcggcacctgtaggtccaggtaccccaccc

SEQ ID NO: 24-101 - skipped (78 entries)

SEQ ID NO: 102 - RNA, 20 nt, synthetic construct
  source 1..20: mol_type = other RNA
cactgcggcccacgaagcga

SEQ ID NO: 103 - RNA, 20 nt, synthetic construct
  source 1..20: mol_type = other RNA
ccccaggatgcattatcccg

SEQ ID NO: 104 - RNA, 20 nt, synthetic construct
  source 1..20: mol_type = other RNA
ccgtgccgtaccttggattg

SEQ ID NO: 105 - RNA, 20 nt, synthetic construct
  source 1..20: mol_type = other RNA
tcggtggtaggtcgccactt

SEQ ID NO: 106 - RNA, 20 nt, synthetic construct
  source 1..20: mol_type = other RNA
aagtcgacatactctcggct

SEQ ID NO: 107 - RNA, 20 nt, synthetic construct
  source 1..20: mol_type = other RNA
cctgcctcacctcacactcg

SEQ ID NO: 108 - RNA, 20 nt, synthetic construct
  source 1..20: mol_type = other RNA
gccgacttacgatttccgag

SEQ ID NO: 109 - RNA, 20 nt, synthetic construct
  source 1..20: mol_type = other RNA
ggagtctgtgttatctggaa

SEQ ID NO: 110 - RNA, 20 nt, synthetic construct
  source 1..20: mol_type = other RNA
gaggcaaaaagaaatcccca

SEQ ID NO: 111 - RNA, 20 nt, synthetic construct
  source 1..20: mol_type = other RNA
gatttctgatgaaaaagacg

SEQ ID NO: 112 - RNA, 20 nt, synthetic construct
  source 1..20: mol_type = other RNA
gctctactggagcgacgagg

SEQ ID NO: 113 - RNA, 20 nt, synthetic construct
  source 1..20: mol_type = other RNA
tcacccaaaaaaatgcacca

SEQ ID NO: 114 - skipped

SEQ ID NO: 115 - RNA, 20 nt, synthetic construct
  source 1..20: mol_type = other RNA
aatctccctcagaaaaaacg

SEQ ID NO: 116 - RNA, 20 nt, synthetic construct
  source 1..20: mol_type = other RNA
agatggtttaaaaaccccca

SEQ ID NO: 117 - RNA, 20 nt, synthetic construct
  source 1..20: mol_type = other RNA
ggacctcaaactgttaaccg

SEQ ID NO: 118 - RNA, 20 nt, synthetic construct
  source 1..20: mol_type = other RNA
gtggatgccttccagaacgt

SEQ ID NO: 119 - RNA, 20 nt, synthetic construct
  source 1..20: mol_type = other RNA
ctccgttcatctggtcaaag

SEQ ID NO: 120 - RNA, 20 nt, synthetic construct
  source 1..20: mol_type = other RNA
ctccgttcatctggtcaaag

SEQ ID NO: 121 - RNA, 20 nt, synthetic construct
  source 1..20: mol_type = other RNA
ctttgaccagatgaacggag

SEQ ID NO: 122 - RNA, 20 nt, synthetic construct
  source 1..20: mol_type = other RNA
ctttgaccagatgaacggag

SEQ ID NO: 123 - RNA, 20 nt, synthetic construct
  source 1..20: mol_type = other RNA
gggaaagatggtcaccgacg

SEQ ID NO: 124 - RNA, 20 nt, synthetic construct
  source 1..20: mol_type = other RNA
gggaaagatggtcaccgacg

SEQ ID NO: 125 - RNA, 20 nt, synthetic construct
  source 1..20: mol_type = other RNA
gtctgacaacatctacccgg

SEQ ID NO: 126 - skipped

SEQ ID NO: 127 - RNA, 20 nt, synthetic construct
  source 1..20: mol_type = other RNA
gtctgacaacatctacccgg

SEQ ID NO: 128 - RNA, 20 nt, synthetic construct
  source 1..20: mol_type = other RNA
aaagacaagcagatccaccc

SEQ ID NO: 129 - RNA, 20 nt, synthetic construct
  source 1..20: mol_type = other RNA
gaaggcgaactttcccaagt

SEQ ID NO: 130 - RNA, 20 nt, synthetic construct
  source 1..20: mol_type = other RNA
taccaccaccccaacgacat

SEQ ID NO: 131 - RNA, 20 nt, synthetic construct
  source 1..20: mol_type = other RNA
tggccgattggtaatcctgg

SEQ ID NO: 132 - RNA, 20 nt, synthetic construct
  source 1..20: mol_type = other RNA
cttactaatgaagtaatccg

SEQ ID NO: 133 - RNA, 20 nt, synthetic construct
  source 1..20: mol_type = other RNA
gagaaagcagtctttaccca

SEQ ID NO: 134 - RNA, 20 nt, synthetic construct
  source 1..20: mol_type = other RNA
ggtctcggagaatgaccgag

SEQ ID NO: 135 - RNA, 20 nt, synthetic construct
  source 1..20: mol_type = other RNA
tgcatggggaggaccagtcg

SEQ ID NO: 136 - RNA, 20 nt, synthetic construct
  source 1..20: mol_type = other RNA
acaggttgccccacgcgttg

SEQ ID NO: 137 - RNA, 20 nt, synthetic construct
  source 1..20: mol_type = other RNA
ggagtttagccagtccaact

SEQ ID NO: 138 - RNA, 20 nt, synthetic construct
  source 1..20: mol_type = other RNA
ggtggcgcaaacgagtagca

SEQ ID NO: 139 - RNA, 20 nt, synthetic construct
  source 1..20: mol_type = other RNA
tagcatttgagctagttcga

SEQ ID NO: 140 - RNA, 20 nt, synthetic construct
  source 1..20: mol_type = other RNA
aagcagagtgcacagaagaa

SEQ ID NO: 141 - RNA, 20 nt, synthetic construct
  source 1..20: mol_type = other RNA
ccgaaatgttgagtgtggtg

SEQ ID NO: 142 - RNA, 20 nt, synthetic construct
  source 1..20: mol_type = other RNA
ctccagagaatttctccccg

SEQ ID NO: 143 - RNA, 20 nt, synthetic construct
  source 1..20: mol_type = other RNA
tctttctagatcccatacag

SEQ ID NO: 144 - RNA, 20 nt, synthetic construct
  source 1..20: mol_type = other RNA
aggtaccctccgacccacca

SEQ ID NO: 145 - RNA, 20 nt, synthetic construct
  source 1..20: mol_type = other RNA
aggtaccctccgacccacca

SEQ ID NO: 146 - RNA, 20 nt, synthetic construct
  source 1..20: mol_type = other RNA
cagggagtgtgtgaactgtg

SEQ ID NO: 147 - RNA, 20 nt, synthetic construct
  source 1..20: mol_type = other RNA
cagggagtgtgtgaactgtg

SEQ ID NO: 148 - RNA, 20 nt, synthetic construct
  source 1..20: mol_type = other RNA
ggagctgtactcgggcacgt

SEQ ID NO: 149 - RNA, 20 nt, synthetic construct
  source 1..20: mol_type = other RNA
ggagctgtactcgggcacgt

SEQ ID NO: 150 - RNA, 20 nt, synthetic construct
  source 1..20: mol_type = other RNA
tccaagacgtccatccacca

SEQ ID NO: 151 - RNA, 20 nt, synthetic construct
  source 1..20: mol_type = other RNA
tccaagacgtccatccacca

SEQ ID NO: 152 - RNA, 20 nt, synthetic construct
  source 1..20: mol_type = other RNA
aagaaacgcctcaagcgcca

SEQ ID NO: 153 - RNA, 20 nt, synthetic construct
  source 1..20: mol_type = other RNA
caggttgtcatgcaccacca

SEQ ID NO: 154 - RNA, 20 nt, synthetic construct
  source 1..20: mol_type = other RNA
ccttccgacccatttcgcgg

SEQ ID NO: 155 - RNA, 20 nt, synthetic construct
  source 1..20: mol_type = other RNA
ctgtacgtgtgcattccgtg

SEQ ID NO: 156 - RNA, 20 nt, synthetic construct
  source 1..20: mol_type = other RNA
aagtgtaccctaactagccg

SEQ ID NO: 157 - RNA, 20 nt, synthetic construct
  source 1..20: mol_type = other RNA
gggtcagcactacttcgaag

SEQ ID NO: 158 - RNA, 20 nt, synthetic construct
  source 1..20: mol_type = other RNA
tatgtgtgaattacgttgtg

SEQ ID NO: 159 - RNA, 20 nt, synthetic construct
  source 1..20: mol_type = other RNA
ttgtcctttagtaaacacta

SEQ ID NO: 160 - RNA, 20 nt, synthetic construct
  source 1..20: mol_type = other RNA
aaagtagtacatatcgactc

SEQ ID NO: 161 - RNA, 20 nt, synthetic construct
  source 1..20: mol_type = other RNA
acactaacgagttcatcacc

SEQ ID NO: 162 - RNA, 20 nt, synthetic construct
  source 1..20: mol_type = other RNA
agttattccagaaactaatg

SEQ ID NO: 163 - RNA, 20 nt, synthetic construct
  source 1..20: mol_type = other RNA
tatgcacctgtcattcagag

SEQ ID NO: 164 - RNA, 20 nt, synthetic construct
  source 1..20: mol_type = other RNA
agaagactaaaggagtatgg

SEQ ID NO: 165 - RNA, 20 nt, synthetic construct
  source 1..20: mol_type = other RNA
cataaacactgaccttgcca

SEQ ID NO: 166 - RNA, 20 nt, synthetic construct
  source 1..20: mol_type = other RNA
tctccctctatgtcatacaa

SEQ ID NO: 167 - RNA, 20 nt, synthetic construct
  source 1..20: mol_type = other RNA
tgactcactgtccactagag

SEQ ID NO: 168 - RNA, 20 nt, synthetic construct
  source 1..20: mol_type = other RNA
ctgcagggaacctccaccat

SEQ ID NO: 169 - RNA, 20 nt, synthetic construct
  source 1..20: mol_type = other RNA
ggatacagggctctacacag

SEQ ID NO: 170 - RNA, 20 nt, synthetic construct
  source 1..20: mol_type = other RNA
tggctttgaatgtggcgtgt

SEQ ID NO: 171 - RNA, 20 nt, synthetic construct
  source 1..20: mol_type = other RNA
ttgtttcgttgtgttccgag

SEQ ID NO: 172 - RNA, 20 nt, synthetic construct
  source 1..20: mol_type = other RNA
acctgatcccaaaacatgga

SEQ ID NO: 173 - RNA, 20 nt, synthetic construct
  source 1..20: mol_type = other RNA
cagcattcggtagaccctga

SEQ ID NO: 174 - RNA, 20 nt, synthetic construct
  source 1..20: mol_type = other RNA
ggatgcatgcggctagacat

SEQ ID NO: 175 - RNA, 20 nt, synthetic construct
  source 1..20: mol_type = other RNA
tacctgcataggaagacacg

SEQ ID NO: 176 - RNA, 20 nt, synthetic construct
  source 1..20: mol_type = other RNA
cagacccgtacaaagtgtac

SEQ ID NO: 177 - RNA, 20 nt, synthetic construct
  source 1..20: mol_type = other RNA
cagacccgtacaaagtgtac

SEQ ID NO: 178 - RNA, 20 nt, synthetic construct
  source 1..20: mol_type = other RNA
cccatgacgtttggaccccg

SEQ ID NO: 179 - RNA, 20 nt, synthetic construct
  source 1..20: mol_type = other RNA
cccatgacgtttggaccccg

SEQ ID NO: 180 - RNA, 20 nt, synthetic construct
  source 1..20: mol_type = other RNA
ctaccgggaaatcctcgtga

SEQ ID NO: 181 - RNA, 20 nt, synthetic construct
  source 1..20: mol_type = other RNA
ctaccgggaaatcctcgtga

SEQ ID NO: 182 - RNA, 20 nt, synthetic construct
  source 1..20: mol_type = other RNA
ctgatcgaccagatcgacag

SEQ ID NO: 183 - RNA, 20 nt, synthetic construct
  source 1..20: mol_type = other RNA
ctgatcgaccagatcgacag

SEQ ID NO: 184 - RNA, 20 nt, synthetic construct
  source 1..20: mol_type = other RNA
gcaatggaacaccaatactc

SEQ ID NO: 185 - RNA, 20 nt, synthetic construct
  source 1..20: mol_type = other RNA
ggatacgtggacccttctgg

SEQ ID NO: 186 - RNA, 20 nt, synthetic construct
  source 1..20: mol_type = other RNA
tacgaacgagttgtatcacc

SEQ ID NO: 187 - RNA, 20 nt, synthetic construct
  source 1..20: mol_type = other RNA
tgtccttcagtggacaacga

SEQ ID NO: 188 - RNA, 20 nt, synthetic construct
  source 1..20: mol_type = other RNA
aatgcacttccacaacacaa

SEQ ID NO: 189 - RNA, 20 nt, synthetic construct
  source 1..20: mol_type = other RNA
aatgtgctgcacataaccct

SEQ ID NO: 190 - RNA, 20 nt, synthetic construct
  source 1..20: mol_type = other RNA
ccacgtgcgagtgcacacga

SEQ ID NO: 191 - RNA, 20 nt, synthetic construct
  source 1..20: mol_type = other RNA
gcacattcgaactcatacag

SEQ ID NO: 192 - RNA, 20 nt, synthetic construct
  source 1..20: mol_type = other RNA
actcgactacggcgtcaccg

SEQ ID NO: 193 - RNA, 20 nt, synthetic construct
  source 1..20: mol_type = other RNA
actcgactacggcgtcaccg

SEQ ID NO: 194 - RNA, 20 nt, synthetic construct
  source 1..20: mol_type = other RNA
cccactccatagaatctccg

SEQ ID NO: 195 - RNA, 20 nt, synthetic construct
  source 1..20: mol_type = other RNA
cccactccatagaatctccg

SEQ ID NO: 196 - RNA, 20 nt, synthetic construct
  source 1..20: mol_type = other RNA
ctgcaactgaaacgcaagcg

SEQ ID NO: 197 - RNA, 20 nt, synthetic construct
  source 1..20: mol_type = other RNA
ctgcaactgaaacgcaagcg

SEQ ID NO: 198 - RNA, 20 nt, synthetic construct
  source 1..20: mol_type = other RNA
gggaccagccaagatcgagg

SEQ ID NO: 199 - RNA, 20 nt, synthetic construct
  source 1..20: mol_type = other RNA
gggaccagccaagatcgagg

SEQ ID NO: 200 - RNA, 20 nt, synthetic construct
  source 1..20: mol_type = other RNA
agacgctatccacgtcgtag

SEQ ID NO: 201 - skipped

SEQ ID NO: 202 - RNA, 20 nt, synthetic construct
  source 1..20: mol_type = other RNA
gcaaatgttacaggtcctgg

SEQ ID NO: 203 - RNA, 20 nt, synthetic construct
  source 1..20: mol_type = other RNA
gcaaatgttacaggtcctgg

SEQ ID NO: 204 - RNA, 20 nt, synthetic construct
  source 1..20: mol_type = other RNA
ggcggggacaagaaggtcat

SEQ ID NO: 205 - RNA, 20 nt, synthetic construct
  source 1..20: mol_type = other RNA
ggcggggacaagaaggtcat

SEQ ID NO: 206 - RNA, 20 nt, synthetic construct
  source 1..20: mol_type = other RNA
gtcttgcaggaatgacacca

SEQ ID NO: 207 - RNA, 20 nt, synthetic construct
  source 1..20: mol_type = other RNA
gtcttgcaggaatgacacca

SEQ ID NO: 208 - RNA, 20 nt, synthetic construct
  source 1..20: mol_type = other RNA
catcctagtcaactcccgtg

SEQ ID NO: 209 - RNA, 20 nt, synthetic construct
  source 1..20: mol_type = other RNA
gcagcagcgaaagcgccttg

SEQ ID NO: 210 - RNA, 20 nt, synthetic construct
  source 1..20: mol_type = other RNA
gggcacaaggtcctacatgt

SEQ ID NO: 211 - RNA, 20 nt, synthetic construct
  source 1..20: mol_type = other RNA
tatggtgcgttctacagcga

SEQ ID NO: 212 - RNA, 20 nt, synthetic construct
  source 1..20: mol_type = other RNA
agagcgtgcagataatgaca

SEQ ID NO: 213 - RNA, 20 nt, synthetic construct
  source 1..20: mol_type = other RNA
agagcgtgcagataatgaca

SEQ ID NO: 214 - RNA, 20 nt, synthetic construct
  source 1..20: mol_type = other RNA
agctggcagaccacaaactg

SEQ ID NO: 215 - RNA, 20 nt, synthetic construct
  source 1..20: mol_type = other RNA
agctggcagaccacaaactg

SEQ ID NO: 216 - RNA, 20 nt, synthetic construct
  source 1..20: mol_type = other RNA
attcttcataccaggaccag

SEQ ID NO: 217 - RNA, 20 nt, synthetic construct
  source 1..20: mol_type = other RNA
attcttcataccaggaccag

SEQ ID NO: 218 - RNA, 20 nt, synthetic construct
  source 1..20: mol_type = other RNA
ccaattcaggacccacacga

SEQ ID NO: 219 - RNA, 20 nt, synthetic construct
  source 1..20: mol_type = other RNA
ccaattcaggacccacacga

SEQ ID NO: 220 - RNA, 20 nt, synthetic construct
  source 1..20: mol_type = other RNA
actacgacctgaatgctgtg

SEQ ID NO: 221 - RNA, 20 nt, synthetic construct
  source 1..20: mol_type = other RNA
gcttccgctacaagtgcgag

SEQ ID NO: 222 - RNA, 20 nt, synthetic construct
  source 1..20: mol_type = other RNA
ggaagatctcatccccaccg

SEQ ID NO: 223 - RNA, 20 nt, synthetic construct
  source 1..20: mol_type = other RNA
tcaatggctacacaggacca

SEQ ID NO: 224 - RNA, 20 nt, synthetic construct
  source 1..20: mol_type = other RNA
atgctaagtacctgtgaaag

SEQ ID NO: 225 - RNA, 20 nt, synthetic construct
  source 1..20: mol_type = other RNA
atgctaagtacctgtgaaag

SEQ ID NO: 226 - RNA, 20 nt, synthetic construct
  source 1..20: mol_type = other RNA
cattgaatatgattgcaagg

SEQ ID NO: 227 - RNA, 20 nt, synthetic construct
  source 1..20: mol_type = other RNA
cattgaatatgattgcaagg

SEQ ID NO: 228 - RNA, 20 nt, synthetic construct
  source 1..20: mol_type = other RNA
taggtgttgatacgagccca

SEQ ID NO: 229 - RNA, 20 nt, synthetic construct
  source 1..20: mol_type = other RNA
taggtgttgatacgagccca

SEQ ID NO: 230 - RNA, 20 nt, synthetic construct
  source 1..20: mol_type = other RNA
tattcatagatctactgaca

SEQ ID NO: 231 - RNA, 20 nt, synthetic construct
  source 1..20: mol_type = other RNA
tattcatagatctactgaca

SEQ ID NO: 232 - RNA, 20 nt, synthetic construct
  source 1..20: mol_type = other RNA
aggttggtgactgtgaacgc

SEQ ID NO: 233 - RNA, 20 nt, synthetic construct
  source 1..20: mol_type = other RNA
agttcatcgacaacaagctg

SEQ ID NO: 234 - RNA, 20 nt, synthetic construct
  source 1..20: mol_type = other RNA
gggctgacactagcagacac

SEQ ID NO: 235 - RNA, 20 nt, synthetic construct
  source 1..20: mol_type = other RNA
tctgttgtggggtctgaacg

SEQ ID NO: 236 - RNA, 20 nt, synthetic construct
  source 1..20: mol_type = other RNA
acattctgtacaatgaacag

SEQ ID NO: 237 - RNA, 20 nt, synthetic construct
  source 1..20: mol_type = other RNA
acattctgtacaatgaacag

SEQ ID NO: 238 - RNA, 20 nt, synthetic construct
  source 1..20: mol_type = other RNA
atcaagcgtgctgaccggcg

SEQ ID NO: 239 - RNA, 20 nt, synthetic construct
  source 1..20: mol_type = other RNA
atcaagcgtgctgaccggcg

SEQ ID NO: 240 - RNA, 20 nt, synthetic construct
  source 1..20: mol_type = other RNA
cagccaggaccacaatgcca

SEQ ID NO: 241 - RNA, 20 nt, synthetic construct
  source 1..20: mol_type = other RNA
cagccaggaccacaatgcca

SEQ ID NO: 242 - RNA, 20 nt, synthetic construct
  source 1..20: mol_type = other RNA
cgtgcacatgaatccccccc

SEQ ID NO: 243 - RNA, 20 nt, synthetic construct
  source 1..20: mol_type = other RNA
cgtgcacatgaatccccccc

SEQ ID NO: 244 - RNA, 20 nt, synthetic construct
  source 1..20: mol_type = other RNA
cagccaggacaacaatgcga

SEQ ID NO: 245 - RNA, 20 nt, synthetic construct
  source 1..20: mol_type = other RNA
gtggccttaatgttctcctg

SEQ ID NO: 246 - RNA, 20 nt, synthetic construct
  source 1..20: mol_type = other RNA
gttcattgtacaatatatgg

SEQ ID NO: 247 - RNA, 20 nt, synthetic construct
  source 1..20: mol_type = other RNA
taagaggtcagaccgtcgtg

SEQ ID NO: 248 - RNA, 20 nt, synthetic construct
  source 1..20: mol_type = other RNA
accggcagcgtcaaacaccc

SEQ ID NO: 249 - RNA, 20 nt, synthetic construct
  source 1..20: mol_type = other RNA
atgaccagaaaaacataaga

SEQ ID NO: 250 - RNA, 20 nt, synthetic construct
  source 1..20: mol_type = other RNA
ccttcatggagaaatgccgt

SEQ ID NO: 251 - RNA, 20 nt, synthetic construct
  source 1..20: mol_type = other RNA
ggtgcagagaaaccggcatg

SEQ ID NO: 252 - RNA, 20 nt, synthetic construct
  source 1..20: mol_type = other RNA
agaacgttcgtggttccgtg

SEQ ID NO: 253 - RNA, 20 nt, synthetic construct
  source 1..20: mol_type = other RNA
atgggcaagaccgctcgccg

SEQ ID NO: 254 - RNA, 20 nt, synthetic construct
  source 1..20: mol_type = other RNA
gttgtgtataactttgtctg

SEQ ID NO: 255 - RNA, 20 nt, synthetic construct
  source 1..20: mol_type = other RNA
taaaagggcgatctaatgaa

SEQ ID NO: 256 - RNA, 20 nt, synthetic construct
  source 1..20: mol_type = other RNA
agatattgaccatgagacag

SEQ ID NO: 257 - RNA, 20 nt, synthetic construct
  source 1..20: mol_type = other RNA
ggagaccatcgagaccacag

SEQ ID NO: 258 - RNA, 20 nt, synthetic construct
  source 1..20: mol_type = other RNA
ggtcaccgacgacccgaccc

SEQ ID NO: 259 - RNA, 20 nt, synthetic construct
  source 1..20: mol_type = other RNA
tgaacaaacgctggtcaccg

SEQ ID NO: 260 - RNA, 20 nt, synthetic construct
  source 1..20: mol_type = other RNA
actgctaaatctcaacagag

SEQ ID NO: 261 - RNA, 20 nt, synthetic construct
  source 1..20: mol_type = other RNA
cttgacagaggcagtcaccg

SEQ ID NO: 262 - RNA, 20 nt, synthetic construct
  source 1..20: mol_type = other RNA
tatgggtacatgttgaacat

SEQ ID NO: 263 - RNA, 20 nt, synthetic construct
  source 1..20: mol_type = other RNA
tgaccagagtgcattacagg

SEQ ID NO: 264 - RNA, 20 nt, synthetic construct
  source 1..20: mol_type = other RNA
cctgcgtgtaccaaatgcag

SEQ ID NO: 265 - RNA, 20 nt, synthetic construct
  source 1..20: mol_type = other RNA
cgggtggctctcaagcgcgg

SEQ ID NO: 266 - RNA, 20 nt, synthetic construct
  source 1..20: mol_type = other RNA
gaaatcgacagtactgacat

SEQ ID NO: 267 - RNA, 20 nt, synthetic construct
  source 1..20: mol_type = other RNA
tggtcagcttggtgtagtcg

SEQ ID NO: 268 - RNA, 20 nt, synthetic construct
  source 1..20: mol_type = other RNA
agccggtccctttcccgtcg

SEQ ID NO: 269 - RNA, 20 nt, synthetic construct
  source 1..20: mol_type = other RNA
cctcagttggcaaggtacct

SEQ ID NO: 270 - RNA, 20 nt, synthetic construct
  source 1..20: mol_type = other RNA
cctctcaatcaaaataaggt

SEQ ID NO: 271 - RNA, 20 nt, synthetic construct
  source 1..20: mol_type = other RNA
cgaaaatctgggctaagtgc

SEQ ID NO: 272 - RNA, 20 nt, synthetic construct
  source 1..20: mol_type = other RNA
atcacacatagcgacgaagt

SEQ ID NO: 273 - RNA, 20 nt, synthetic construct
  source 1..20: mol_type = other RNA
cagagcatctctagctaacg

SEQ ID NO: 274 - RNA, 20 nt, synthetic construct
  source 1..20: mol_type = other RNA
ctaactctgctacccaagtg

SEQ ID NO: 275 - RNA, 20 nt, synthetic construct
  source 1..20: mol_type = other RNA
taatgttaatccgagaacgg

SEQ ID NO: 276 - RNA, 20 nt, synthetic construct
  source 1..20: mol_type = other RNA
aaggaaagagtctactgtcg

SEQ ID NO: 277 - RNA, 20 nt, synthetic construct
  source 1..20: mol_type = other RNA
agatgtgagtggatctatcg

SEQ ID NO: 278 - RNA, 20 nt, synthetic construct
  source 1..20: mol_type = other RNA
ccttacctgaatcaatactg

SEQ ID NO: 279 - RNA, 20 nt, synthetic construct
  source 1..20: mol_type = other RNA
gttatctataagacaccttg

SEQ ID NO: 280 - RNA, 20 nt, synthetic construct
  source 1..20: mol_type = other RNA
acatcgactgctggacaatg

SEQ ID NO: 281 - RNA, 20 nt, synthetic construct
  source 1..20: mol_type = other RNA
cagtgagtagtgccaaacca

SEQ ID NO: 282 - RNA, 20 nt, synthetic construct
  source 1..20: mol_type = other RNA
gtggcgtactgcacgtgtcg

SEQ ID NO: 283 - RNA, 20 nt, synthetic construct
  source 1..20: mol_type = other RNA
ttcacattatgaccaacacc

SEQ ID NO: 284 - RNA, 20 nt, synthetic construct
  source 1..20: mol_type = other RNA
gaaaatgaatggctcccaca

SEQ ID NO: 285 - RNA, 20 nt, synthetic construct
  source 1..20: mol_type = other RNA
gatggcttggtccatcacgt

SEQ ID NO: 286 - RNA, 20 nt, synthetic construct
  source 1..20: mol_type = other RNA
ggggcctcattcacccagaa

SEQ ID NO: 287 - RNA, 20 nt, synthetic construct
  source 1..20: mol_type = other RNA
tccaagagtgacagagtcgt

SEQ ID NO: 288 - RNA, 20 nt, synthetic construct
  source 1..20: mol_type = other RNA
cgatccaaatttgaacgccg

SEQ ID NO: 289 - RNA, 20 nt, synthetic construct
  source 1..20: mol_type = other RNA
gagcaaactgcgttatacag

SEQ ID NO: 290 - RNA, 20 nt, synthetic construct
  source 1..20: mol_type = other RNA
ttaccccagaaccagacgga

SEQ ID NO: 291 - RNA, 20 nt, synthetic construct
  source 1..20: mol_type = other RNA
tttgtgcagttatgccagca

SEQ ID NO: 292 - RNA, 20 nt, synthetic construct
  source 1..20: mol_type = other RNA
aagagagctattcgtctggg

SEQ ID NO: 293 - RNA, 20 nt, synthetic construct
  source 1..20: mol_type = other RNA
agctgggtcggagaacaaca

SEQ ID NO: 294 - RNA, 20 nt, synthetic construct
  source 1..20: mol_type = other RNA
gccaggacagcagacaagtg

SEQ ID NO: 295 - RNA, 20 nt, synthetic construct
  source 1..20: mol_type = other RNA
tgcttaccccataacttgtg

SEQ ID NO: 296 - RNA, 20 nt, synthetic construct
  source 1..20: mol_type = other RNA
aagatgaactgcgatgtgtg

SEQ ID NO: 297 - RNA, 20 nt, synthetic construct
  source 1..20: mol_type = other RNA
caagcagagaagttcccttg

SEQ ID NO: 298 - RNA, 20 nt, synthetic construct
  source 1..20: mol_type = other RNA
gctcatacagacccgcatga

SEQ ID NO: 299 - RNA, 20 nt, synthetic construct
  source 1..20: mol_type = other RNA
ggacagattagcaagcaatg

SEQ ID NO: 300 - RNA, 20 nt, synthetic construct
  source 1..20: mol_type = other RNA
aactgggatagattgcaccc

SEQ ID NO: 301 - RNA, 20 nt, synthetic construct
  source 1..20: mol_type = other RNA
agccatctgaccatctagct

SEQ ID NO: 302 - RNA, 20 nt, synthetic construct
  source 1..20: mol_type = other RNA
atggttatatgtgacaagtg

SEQ ID NO: 303 - RNA, 20 nt, synthetic construct
  source 1..20: mol_type = other RNA
gcagacattaccctatagtg

SEQ ID NO: 304 - RNA, 20 nt, synthetic construct
  source 1..20: mol_type = other RNA
actggaatcaacaacaatcg

SEQ ID NO: 305 - RNA, 20 nt, synthetic construct
  source 1..20: mol_type = other RNA
atgccacaatcacttaaggt

SEQ ID NO: 306 - RNA, 20 nt, synthetic construct
  source 1..20: mol_type = other RNA
gcctcaccgttagaccagaa

SEQ ID NO: 307 - RNA, 20 nt, synthetic construct
  source 1..20: mol_type = other RNA
ggtttgggagtatatgaccg

SEQ ID NO: 308 - RNA, 20 nt, synthetic construct
  source 1..20: mol_type = other RNA
acctggtgtggacccacgcg

SEQ ID NO: 309 - RNA, 20 nt, synthetic construct
  source 1..20: mol_type = other RNA
cctcgaactgcaccataggt

SEQ ID NO: 310 - RNA, 20 nt, synthetic construct
  source 1..20: mol_type = other RNA
gccattgatctgatgtacgg

SEQ ID NO: 311 - RNA, 20 nt, synthetic construct
  source 1..20: mol_type = other RNA
tggggctctgcatctcacag

SEQ ID NO: 312 - RNA, 20 nt, synthetic construct
  source 1..20: mol_type = other RNA
agatgtattccgcatagtca

SEQ ID NO: 313 - RNA, 20 nt, synthetic construct
  source 1..20: mol_type = other RNA
cacttagcgtgataaacccg

SEQ ID NO: 314 - RNA, 20 nt, synthetic construct
  source 1..20: mol_type = other RNA
ctcttccgcccaaacttccg

SEQ ID NO: 315 - RNA, 20 nt, synthetic construct
  source 1..20: mol_type = other RNA
ggagcgcggcatatccgaca

SEQ ID NO: 316 - RNA, 20 nt, synthetic construct
  source 1..20: mol_type = other RNA
cacaagcactcgccaccaag

SEQ ID NO: 317 - RNA, 20 nt, synthetic construct
  source 1..20: mol_type = other RNA
cgtggttgagggaaaatcca

SEQ ID NO: 318 - RNA, 20 nt, synthetic construct
  source 1..20: mol_type = other RNA
gtgatactctgatcaccgta

SEQ ID NO: 319 - RNA, 20 nt, synthetic construct
  source 1..20: mol_type = other RNA
tgtccctaaatgcccccaaa

SEQ ID NO: 320 - RNA, 20 nt, synthetic construct
  source 1..20: mol_type = other RNA
agaggaggagacacatgtcg

SEQ ID NO: 321 - RNA, 20 nt, synthetic construct
  source 1..20: mol_type = other RNA
agaggaggagacacatgtcg

SEQ ID NO: 322 - RNA, 20 nt, synthetic construct
  source 1..20: mol_type = other RNA
catacaccatgtccatagag

SEQ ID NO: 323 - RNA, 20 nt, synthetic construct
  source 1..20: mol_type = other RNA
catacaccatgtccatagag

SEQ ID NO: 324 - RNA, 20 nt, synthetic construct
  source 1..20: mol_type = other RNA
gccttctgacaattcagccc

SEQ ID NO: 325 - RNA, 20 nt, synthetic construct
  source 1..20: mol_type = other RNA
gccttctgacaattcagccc

SEQ ID NO: 326 - RNA, 20 nt, synthetic construct
  source 1..20: mol_type = other RNA
gttctgtagacttcacatgc

SEQ ID NO: 327 - RNA, 20 nt, synthetic construct
  source 1..20: mol_type = other RNA
gttctgtagacttcacatgc

SEQ ID NO: 328 - RNA, 20 nt, synthetic construct
  source 1..20: mol_type = other RNA
cgggacacgtctacttggtg

SEQ ID NO: 329 - RNA, 20 nt, synthetic construct
  source 1..20: mol_type = other RNA
gcagaacgaggctgccctag

SEQ ID NO: 330 - RNA, 20 nt, synthetic construct
  source 1..20: mol_type = other RNA
gcggaccagtgtacagcacg

SEQ ID NO: 331 - RNA, 20 nt, synthetic construct
  source 1..20: mol_type = other RNA
taaggtgaggactttgcaca

SEQ ID NO: 332 - RNA, 20 nt, synthetic construct
  source 1..20: mol_type = other RNA
ccggggctggtacttatgga

SEQ ID NO: 333 - RNA, 20 nt, synthetic construct
  source 1..20: mol_type = other RNA
cctggggtctccctacccgg

SEQ ID NO: 334 - RNA, 20 nt, synthetic construct
  source 1..20: mol_type = other RNA
gcggtaccagagcggcaagt

SEQ ID NO: 335 - RNA, 20 nt, synthetic construct
  source 1..20: mol_type = other RNA
taaacttggaccacaacagg

SEQ ID NO: 336 - RNA, 20 nt, synthetic construct
  source 1..20: mol_type = other RNA
acccaggcatcatccgacaa

SEQ ID NO: 337 - RNA, 20 nt, synthetic construct
  source 1..20: mol_type = other RNA
acccaggcatcatccgacaa

SEQ ID NO: 338 - RNA, 20 nt, synthetic construct
  source 1..20: mol_type = other RNA
cccacccacagggatcaacg

SEQ ID NO: 339 - RNA, 20 nt, synthetic construct
  source 1..20: mol_type = other RNA
cccacccacagggatcaacg

SEQ ID NO: 340 - RNA, 20 nt, synthetic construct
  source 1..20: mol_type = other RNA
cctacttaggcactgccagg

SEQ ID NO: 341 - RNA, 20 nt, synthetic construct
  source 1..20: mol_type = other RNA
cctacttaggcactgccagg

SEQ ID NO: 342 - RNA, 20 nt, synthetic construct
  source 1..20: mol_type = other RNA
gagggtgccaccatgactag

SEQ ID NO: 343 - RNA, 20 nt, synthetic construct
  source 1..20: mol_type = other RNA
gagggtgccaccatgactag

SEQ ID NO: 344 - RNA, 20 nt, synthetic construct
  source 1..20: mol_type = other RNA
cacggaccctgatagcatga

SEQ ID NO: 345 - RNA, 20 nt, synthetic construct
  source 1..20: mol_type = other RNA
cacggaccctgatagcatga

SEQ ID NO: 346 - RNA, 20 nt, synthetic construct
  source 1..20: mol_type = other RNA
catcgctcaggagatatacg

SEQ ID NO: 347 - RNA, 20 nt, synthetic construct
  source 1..20: mol_type = other RNA
catcgctcaggagatatacg

SEQ ID NO: 348 - RNA, 20 nt, synthetic construct
  source 1..20: mol_type = other RNA
gcagccgaatcgccaaccgc

SEQ ID NO: 349 - RNA, 20 nt, synthetic construct
  source 1..20: mol_type = other RNA
gcagccgaatcgccaaccgc

SEQ ID NO: 350 - RNA, 20 nt, synthetic construct
  source 1..20: mol_type = other RNA
gcttcgcagcctgctaacca

SEQ ID NO: 351 - RNA, 20 nt, synthetic construct
  source 1..20: mol_type = other RNA
gcttcgcagcctgctaacca

SEQ ID NO: 352 - RNA, 20 nt, synthetic construct
  source 1..20: mol_type = other RNA
caggcagcctcaacatatcg

SEQ ID NO: 353 - RNA, 20 nt, synthetic construct
  source 1..20: mol_type = other RNA
cctccgcaatgacattcaag

SEQ ID NO: 354 - RNA, 20 nt, synthetic construct
  source 1..20: mol_type = other RNA
taggaggccaaaagacacgg

SEQ ID NO: 355 - RNA, 20 nt, synthetic construct
  source 1..20: mol_type = other RNA
ttcctgcatgatctgaacca

SEQ ID NO: 356 - RNA, 20 nt, synthetic construct
  source 1..20: mol_type = other RNA
acatgaatgcagaggattcg

SEQ ID NO: 357 - RNA, 20 nt, synthetic construct
  source 1..20: mol_type = other RNA
agggcccggaggaacaacag

SEQ ID NO: 358 - RNA, 20 nt, synthetic construct
  source 1..20: mol_type = other RNA
ccagccgatgcacaaaacgt

SEQ ID NO: 359 - RNA, 20 nt, synthetic construct
  source 1..20: mol_type = other RNA
tgtggagcactcataccgaa

SEQ ID NO: 360 - RNA, 20 nt, synthetic construct
  source 1..20: mol_type = other RNA
agcaagtcgtgcgagttctg

SEQ ID NO: 361 - RNA, 20 nt, synthetic construct
  source 1..20: mol_type = other RNA
ccagcagctcgctcacgccg

SEQ ID NO: 362 - RNA, 20 nt, synthetic construct
  source 1..20: mol_type = other RNA
ccgccatggacttctcgcgg

SEQ ID NO: 363 - RNA, 20 nt, synthetic construct
  source 1..20: mol_type = other RNA
tcagggtgagggtcagacgg

SEQ ID NO: 364 - RNA, 20 nt, synthetic construct
  source 1..20: mol_type = other RNA
acactggaacatatcaagac

SEQ ID NO: 365 - RNA, 20 nt, synthetic construct
  source 1..20: mol_type = other RNA
gacaaatgcaatgaaaactg

SEQ ID NO: 366 - RNA, 20 nt, synthetic construct
  source 1..20: mol_type = other RNA
ggacatcgtgtaccgcacca

SEQ ID NO: 367 - RNA, 20 nt, synthetic construct
  source 1..20: mol_type = other RNA
ggccgcccgggaagtcaaca

SEQ ID NO: 368 - RNA, 20 nt, synthetic construct
  source 1..20: mol_type = other RNA
agatccaccaggatggcacg

SEQ ID NO: 369 - RNA, 20 nt, synthetic construct
  source 1..20: mol_type = other RNA
ccccaccggcacctaccacg

SEQ ID NO: 370 - RNA, 20 nt, synthetic construct
  source 1..20: mol_type = other RNA
ctgcattccaggtcagtctg

SEQ ID NO: 371 - RNA, 20 nt, synthetic construct
  source 1..20: mol_type = other RNA
gctgaccacaccaacctacg

SEQ ID NO: 372 - RNA, 20 nt, synthetic construct
  source 1..20: mol_type = other RNA
gaaaagacagtgataagtcc

SEQ ID NO: 373 - RNA, 20 nt, synthetic construct
  source 1..20: mol_type = other RNA
gaaccaaactcacacctgct

SEQ ID NO: 374 - RNA, 20 nt, synthetic construct
  source 1..20: mol_type = other RNA
taggtggtaactgctccgaa

SEQ ID NO: 375 - RNA, 20 nt, synthetic construct
  source 1..20: mol_type = other RNA
tcgagcacctcagcacatca

SEQ ID NO: 376 - RNA, 20 nt, synthetic construct
  source 1..20: mol_type = other RNA
atcccgaacctgcattctga

SEQ ID NO: 377 - RNA, 20 nt, synthetic construct
  source 1..20: mol_type = other RNA
caccgcttcagtgcaacacg

SEQ ID NO: 378 - RNA, 20 nt, synthetic construct
  source 1..20: mol_type = other RNA
ccaaggaaaaaactaacgag

SEQ ID NO: 379 - RNA, 20 nt, synthetic construct
  source 1..20: mol_type = other RNA
gcggctcctagaccggcagg

SEQ ID NO: 380-401 - skipped (22 entries)

SEQ ID NO: 402 - RNA, 20 nt, synthetic construct
  source 1..20: mol_type = other RNA
atttcttcccctcaccacag

SEQ ID NO: 403 - RNA, 20 nt, synthetic construct
  source 1..20: mol_type = other RNA
atcctcacgatgaaccccag

SEQ ID NO: 404 - RNA, 20 nt, synthetic construct
  source 1..20: mol_type = other RNA
gttgaatagagtgcctgctg

SEQ ID NO: 405 - RNA, 20 nt, synthetic construct
  source 1..20: mol_type = other RNA
caggtgtttaacagatgtca

SEQ ID NO: 406 - RNA, 20 nt, synthetic construct
  source 1..20: mol_type = other RNA
tatagtccagtatagagggg

SEQ ID NO: 407 - RNA, 20 nt, synthetic construct
  source 1..20: mol_type = other RNA
tgaatgctgagtgtacaatg

SEQ ID NO: 408 - RNA, 20 nt, synthetic construct
  source 1..20: mol_type = other RNA
tcagatccattctctccgtg

SEQ ID NO: 409 - RNA, 20 nt, synthetic construct
  source 1..20: mol_type = other RNA
tgtctcccctctactaaatg

SEQ ID NO: 410 - RNA, 20 nt, synthetic construct
  source 1..20: mol_type = other RNA
actgtcgcatcttacagaca

SEQ ID NO: 411 - RNA, 20 nt, synthetic construct
  source 1..20: mol_type = other RNA
tgttaggaaccacagacctg

SEQ ID NO: 412 - RNA, 20 nt, synthetic construct
  source 1..20: mol_type = other RNA
cacagtgctaggttccagca

SEQ ID NO: 413 - RNA, 20 nt, synthetic construct
  source 1..20: mol_type = other RNA
agtggtcctaagatgtcaca

SEQ ID NO: 414 - RNA, 20 nt, synthetic construct
  source 1..20: mol_type = other RNA
tcagatccattctctccgtg

SEQ ID NO: 415 - RNA, 20 nt, synthetic construct
  source 1..20: mol_type = other RNA
tgtctcccctctactaaatg

SEQ ID NO: 416 - RNA, 20 nt, synthetic construct
  source 1..20: mol_type = other RNA
actgtcgcatcttacagaca

SEQ ID NO: 417 - RNA, 20 nt, synthetic construct
  source 1..20: mol_type = other RNA
tgttaggaaccacagacctg

SEQ ID NO: 418 - RNA, 20 nt, synthetic construct
  source 1..20: mol_type = other RNA
cacagtgctaggttccagca

SEQ ID NO: 419 - RNA, 20 nt, synthetic construct
  source 1..20: mol_type = other RNA
agtggtcctaagatgtcaca

SEQ ID NO: 420 - RNA, 20 nt, synthetic construct
  source 1..20: mol_type = other RNA
ccccctccaaatttccattg

SEQ ID NO: 421 - RNA, 20 nt, synthetic construct
  source 1..20: mol_type = other RNA
gataacgctgactttccggg

SEQ ID NO: 422 - RNA, 20 nt, synthetic construct
  source 1..20: mol_type = other RNA
ccagccccacaagtaccacg

SEQ ID NO: 423 - RNA, 20 nt, synthetic construct
  source 1..20: mol_type = other RNA
cccctctcattgacacacca

SEQ ID NO: 424 - RNA, 20 nt, synthetic construct
  source 1..20: mol_type = other RNA
acttgaatcagaatactaca

SEQ ID NO: 425 - RNA, 20 nt, synthetic construct
  source 1..20: mol_type = other RNA
atttcttcccctcaccacag

SEQ ID NO: 426 - RNA, 20 nt, synthetic construct
  source 1..20: mol_type = other RNA
atcctcacgatgaaccccag

SEQ ID NO: 427 - RNA, 20 nt, synthetic construct
  source 1..20: mol_type = other RNA
gttgaatagagtgcctgctg

SEQ ID NO: 428 - RNA, 20 nt, synthetic construct
  source 1..20: mol_type = other RNA
caggtgtttaacagatgtca

SEQ ID NO: 429 - RNA, 20 nt, synthetic construct
  source 1..20: mol_type = other RNA
tcagatccattctctccgtg

SEQ ID NO: 430 - RNA, 20 nt, synthetic construct
  source 1..20: mol_type = other RNA
tgtctcccctctactaaatg

SEQ ID NO: 431 - RNA, 20 nt, synthetic construct
  source 1..20: mol_type = other RNA
tcagatccattctctccgtg

SEQ ID NO: 432 - RNA, 20 nt, synthetic construct
  source 1..20: mol_type = other RNA
actgtcgcatcttacagaca

SEQ ID NO: 433 - RNA, 20 nt, synthetic construct
  source 1..20: mol_type = other RNA
ccccctccaaatttccattg

SEQ ID NO: 434 - RNA, 20 nt, synthetic construct
  source 1..20: mol_type = other RNA
ccagccccacaagtaccacg

SEQ ID NO: 435 - RNA, 20 nt, synthetic construct
  source 1..20: mol_type = other RNA
gataacgctgactttccggg

SEQ ID NO: 436 - RNA, 20 nt, synthetic construct
  source 1..20: mol_type = other RNA
gttgaatagagtgcctgctg

SEQ ID NO: 437 - RNA, 20 nt, synthetic construct
  source 1..20: mol_type = other RNA
caggtgtttaacagatgtca

SEQ ID NO: 438 - RNA, 20 nt, synthetic construct
  source 1..20: mol_type = other RNA
tatagtccagtatagagggg

SEQ ID NO: 439 - RNA, 20 nt, synthetic construct
  source 1..20: mol_type = other RNA
tgaatgctgagtgtacaatg

SEQ ID NO: 440 - RNA, 20 nt, synthetic construct
  source 1..20: mol_type = other RNA
cacagtgctaggttccagca

SEQ ID NO: 441 - RNA, 20 nt, synthetic construct
  source 1..20: mol_type = other RNA
agtggtcctaagatgtcaca

SEQ ID NO: 442 - RNA, 20 nt, synthetic construct
  source 1..20: mol_type = other RNA
tgttaggaaccacagacctg

SEQ ID NO: 443 - RNA, 20 nt, synthetic construct
  source 1..20: mol_type = other RNA
cacagtgctaggttccagca

SEQ ID NO: 444 - RNA, 20 nt, synthetic construct
  source 1..20: mol_type = other RNA
ccagccccacaagtaccacg

SEQ ID NO: 445 - RNA, 20 nt, synthetic construct
  source 1..20: mol_type = other RNA
acttgaatcagaatactaca

SEQ ID NO: 446 - RNA, 19 nt, synthetic construct
  source 1..19: mol_type = other RNA
cccctctcattgacacacc

SEQ ID NO: 447-449 - skipped (3 entries)

SEQ ID NO: 450 - RNA, 2159 nt, Homo sapiens
  source 1..2159: mol_type = mRNA
agtttcccacaagccaggctgatccttttctgtcagtccacttcaccaagcctgcccttggacaaggacccgatgcccaaccccaggcctggcaagccctcggccccttccttggcccttggcccatccccaggagcctcgcccagctggagggctgcacccaaagcctcagacctgctgggggcccggggcccagggggaaccttccagggccgagatcttcgaggcggggcccatgcctcctcttcttccttgaaccccatgccaccatcgcagctgcagctctcaacggtggatgcccacgcccggacccctgtgctgcaggtgcaccccctggagagcccagccatgatcagcctcacaccacccaccaccgccactggggtcttctccctcaaggcccggcctggcctcccacctgggatcaacgtggccagcctggaatgggtgtccagggagccggcactgctctgcaccttcccaaatcccagtgcacccaggaaggacagcaccctttcggctgtgccccagagctcctacccactgctggcaaatggtgtctgcaagtggcccggatgtgagaaggtcttcgaagagccagaggacttcctcaagcactgccaggcggaccatcttctggatgagaagggcagggcacaatgtctcctccagagagagatggtacagtctctggagcagcagctggtgctggagaaggagaagctgagtgccatgcaggcccacctggctgggaaaatggcactgaccaaggcttcatctgtggcatcatccgacaagggctcctgctgcatcgtagctgctggcagccaaggccctgtcgtcccagcctggtctggcccccgggaggcccctgacagcctgtttgctgtccggaggcacctgtggggtagccatggaaacagcacattcccagagttcctccacaacatggactacttcaagttccacaacatgcgaccccctttcacctacgccacgctcatccgctgggccatcctggaggctccagagaagcagcggacactcaatgagatctaccactggttcacacgcatgtttgccttcttcagaaaccatcctgccacctggaagaacgccatccgccacaacctgagtctgcacaagtgctttgtgcgggtggagagcgagaagggggctgtgtggaccgtggatgagctggagttccgcaagaaacggagccagaggcccagcaggtgttccaaccctacacctggcccctgacctcaagatcaaggaaaggaggatggacgaacaggggccaaactggtgggaggcagaggtggtgggggcagggatgataggccctggatgtgcccacagggaccaagaagtgaggtttccactgtcttgcctgccagggcccctgttcccccgctggcagccaccccctcccccatcatatcctttgccccaaggctgctcagaggggccccggtcctggccccagcccccacctccgccccagacacaccccccagtcgagccctgcagccaaacagagccttcacaaccagccacacagagcctgcctcagctgctcgcacagattacttcagggctggaaaagtcacacagacacacaaaatgtcacaatcctgtccctcactcaacacaaaccccaaaacacagagagcctgcctcagtacactcaaacaacctcaaagctgcatcatcacacaatcacacacaagcacagccctgacaacccacacaccccaaggcacgcacccacagccagcctcagggcccacaggggcactgtcaacacaggggtgtgcccagaggcctacacagaagcagcgtcagtaccctcaggatctgaggtcccaacacgtgctcgctcacacacacggcctgttagaattcacctgtgtatctcacgcatatgcacacgcacagccccccagtgggtctcttgagtcccgtgcagacacacacagccacacacactgccttgccaaaaataccccgtgtctcccctgccactcacctcactcccattccctgagccctgatccatgcctcagcttagactgcagaggaactactcatttatttgggatccaaggcccccaacccacagtaccgtccccaataaactgcagccgagctcccca

SEQ ID NO: 451 - protein, 431 aa, Homo sapiens
MPNPRPGKPSAPSLALGPSPGASPSWRAAPKASDLLGARGPGGTFQGRDLRGGAHASSSSLNPMPPSQLQLPTLPLVMVAPSGARLGPLPHLQALLQDRPHFMHQLSTVDAHARTPVLQVHPLESPAMISLTPPTTATGVFSLKARPGLPPGINVASLEWVSREPALLCTFPNPSAPRKDSTLSAVPQSSYPLLANGVCKWPGCEKVFEEPEDFLKHCQADHLLDEKGRAQCLLQREMVQSLEQQLVLEKEKLSAMQAHLAGKMALTKASSVASSDKGSCCIVAAGSQGPVVPAWSGPREAPDSLFAVRRHLWGSHGNSTFPEFLHNMDYFKFHNMRPPFTYATLIRWAILEAPEKQRTLNEIYHWFTRMFAFFRNHPATWKNAIRHNLSLHKCFVRVESEKGAVWTVDELEFRKKRSQRPSRCSNPTPGP